SEQUENCE LISTING (484 sequences)

SEQ ID NO: 1 - RNA, 23 nt, Homo sapiens
  source 1..23: mol_type = genomic RNA
agggacttttgggggcagatgtg

SEQ ID NO: 2 - RNA, 22 nt, Homo sapiens
  source 1..22: mol_type = genomic RNA
agggactttcaggggcagctgt

SEQ ID NO: 3 - RNA, 22 nt, Homo sapiens
  source 1..22: mol_type = genomic RNA
ctatacaacctactgccttccc

SEQ ID NO: 4 - RNA, 22 nt, Homo sapiens
  source 1..22: mol_type = genomic RNA
ctatacaatctattgccttccc

SEQ ID NO: 5 - RNA, 23 nt, Homo sapiens
  source 1..23: mol_type = genomic RNA
gagggtcttgggagggatgtgac

SEQ ID NO: 6 - RNA, 22 nt, Homo sapiens
  source 1..22: mol_type = genomic RNA
atatacagggggagactcttat

SEQ ID NO: 7 - RNA, 22 nt, Homo sapiens
  source 1..22: mol_type = genomic RNA
atatacagggggagactctcat

SEQ ID NO: 8 - RNA, 21 nt, Homo sapiens
  source 1..21: mol_type = genomic RNA
tggcagggaggctgggagggg

SEQ ID NO: 9 - RNA, 21 nt, Homo sapiens
  source 1..21: mol_type = genomic RNA
ccccacctcctctctcctcag

SEQ ID NO: 10 - RNA, 22 nt, Homo sapiens
  source 1..22: mol_type = genomic RNA
tgagcccctgtgccgcccccag

SEQ ID NO: 11 - RNA, 22 nt, Homo sapiens
  source 1..22: mol_type = genomic RNA
gtgggtacggcccagtgggggg

SEQ ID NO: 12 - RNA, 20 nt, Homo sapiens
  source 1..20: mol_type = genomic RNA
cgtgccacccttttccccag

SEQ ID NO: 13 - RNA, 20 nt, Homo sapiens
  source 1..20: mol_type = genomic RNA
tcacacctgcctcgcccccc

SEQ ID NO: 14 - RNA, 22 nt, Homo sapiens
  source 1..22: mol_type = genomic RNA
agtgggaggccagggcacggca

SEQ ID NO: 15 - RNA, 22 nt, Homo sapiens
  source 1..22: mol_type = genomic RNA
tcggcctgaccacccaccccac

SEQ ID NO: 16 - RNA, 21 nt, Homo sapiens
  source 1..21: mol_type = genomic RNA
tccttctgctccgtcccccag

SEQ ID NO: 17 - RNA, 20 nt, Homo sapiens
  source 1..20: mol_type = genomic RNA
cttcctcgtctgtctgcccc

SEQ ID NO: 18 - RNA, 22 nt, Homo sapiens
  source 1..22: mol_type = genomic RNA
acccgtcccgttcgtccccgga

SEQ ID NO: 19 - RNA, 22 nt, Homo sapiens
  source 1..22: mol_type = genomic RNA
acaggtgaggttcttgggagcc

SEQ ID NO: 20 - RNA, 21 nt, Homo sapiens
  source 1..21: mol_type = genomic RNA
cctgttgaagtgtaatcccca

SEQ ID NO: 21 - RNA, 17 nt, Homo sapiens
  source 1..17: mol_type = genomic RNA
gtgggggagaggctgtc

SEQ ID NO: 22 - RNA, 22 nt, Homo sapiens
  source 1..22: mol_type = genomic RNA
aagcccttaccccaaaaagtat

SEQ ID NO: 23 - RNA, 22 nt, Homo sapiens
  source 1..22: mol_type = genomic RNA
aagcccttaccccaaaaagcat

SEQ ID NO: 24 - RNA, 22 nt, Homo sapiens
  source 1..22: mol_type = genomic RNA
tctcactgtagcctcgaacccc

SEQ ID NO: 25 - RNA, 22 nt, Homo sapiens
  source 1..22: mol_type = genomic RNA
tcaaaactgaggggcattttct

SEQ ID NO: 26 - RNA, 22 nt, Homo sapiens
  source 1..22: mol_type = genomic RNA
tttggtccccttcaaccagctg

SEQ ID NO: 27 - RNA, 22 nt, Homo sapiens
  source 1..22: mol_type = genomic RNA
tttggtccccttcaaccagcta

SEQ ID NO: 28 - RNA, 22 nt, Homo sapiens
  source 1..22: mol_type = genomic RNA
tgtgactggttgaccagagggg

SEQ ID NO: 29 - RNA, 22 nt, Homo sapiens
  source 1..22: mol_type = genomic RNA
tgccctaaatgccccttctggc

SEQ ID NO: 30 - RNA, 22 nt, Homo sapiens
  source 1..22: mol_type = genomic RNA
gctgcgcttggatttcgtcccc

SEQ ID NO: 31 - RNA, 23 nt, Homo sapiens
  source 1..23: mol_type = genomic RNA
cccagtgttcagactacctgttc

SEQ ID NO: 32 - RNA, 23 nt, Homo sapiens
  source 1..23: mol_type = genomic RNA
cccagtgtttagactatctgttc

SEQ ID NO: 33 - RNA, 22 nt, Homo sapiens
  source 1..22: mol_type = genomic RNA
agcccctgcccaccgcacactg

SEQ ID NO: 34 - RNA, 21 nt, Homo sapiens
  source 1..21: mol_type = genomic RNA
cctcccatgccaagaactccc

SEQ ID NO: 35 - RNA, 24 nt, Homo sapiens
  source 1..24: mol_type = genomic RNA
acacacttacccgtagagattcta

SEQ ID NO: 36 - RNA, 22 nt, Homo sapiens
  source 1..22: mol_type = genomic RNA
tgtcagtttgtcaaatacccca

SEQ ID NO: 37 - RNA, 21 nt, Homo sapiens
  source 1..21: mol_type = genomic RNA
agggccccccctcaatcctgt

SEQ ID NO: 38 - RNA, 21 nt, Homo sapiens
  source 1..21: mol_type = genomic RNA
tctggctgctatggccccctc

SEQ ID NO: 39 - RNA, 22 nt, Homo sapiens
  source 1..22: mol_type = genomic RNA
caacctcgaggatctccccagc

SEQ ID NO: 40 - RNA, 21 nt, Homo sapiens
  source 1..21: mol_type = genomic RNA
tccctacccctccactcccca

SEQ ID NO: 41 - RNA, 25 nt, Homo sapiens
  source 1..25: mol_type = genomic RNA
tgccccatctgtgccctgggtagga

SEQ ID NO: 42 - RNA, 20 nt, Homo sapiens
  source 1..20: mol_type = genomic RNA
tctggccagctacgtcccca

SEQ ID NO: 43 - RNA, 23 nt, Homo sapiens
  source 1..23: mol_type = genomic RNA
gggggggcaggaggggctcaggg

SEQ ID NO: 44 - RNA, 21 nt, Homo sapiens
  source 1..21: mol_type = genomic RNA
gcccctgggcctatcctagaa

SEQ ID NO: 45 - RNA, 23 nt, Homo sapiens
  source 1..23: mol_type = genomic RNA
tcccccaggtgtgattctgattt

SEQ ID NO: 46 - RNA, 23 nt, Homo sapiens
  source 1..23: mol_type = genomic RNA
ccacttggatctgaaggctgccc

SEQ ID NO: 47 - RNA, 23 nt, Homo sapiens
  source 1..23: mol_type = genomic RNA
agggacttttgggggcagatgtg

SEQ ID NO: 48 - RNA, 22 nt, Homo sapiens
  source 1..22: mol_type = genomic RNA
gaaggcagcagtgctcccctgt

SEQ ID NO: 49 - RNA, 20 nt, Homo sapiens
  source 1..20: mol_type = genomic RNA
actcaaactgtgggggcact

SEQ ID NO: 50 - RNA, 23 nt, Homo sapiens
  source 1..23: mol_type = genomic RNA
aagtgcccccacagtttgagtgc

SEQ ID NO: 51 - RNA, 23 nt, Homo sapiens
  source 1..23: mol_type = genomic RNA
gcgacgagcccctcgcacaaacc

SEQ ID NO: 52 - RNA, 23 nt, Homo sapiens
  source 1..23: mol_type = genomic RNA
tagcccccaggcttcacttggcg

SEQ ID NO: 53 - RNA, 22 nt, Homo sapiens
  source 1..22: mol_type = genomic RNA
gaatgttgctcggtgaacccct

SEQ ID NO: 54 - RNA, 21 nt, Homo sapiens
  source 1..21: mol_type = genomic RNA
gcaggcacagacagccctggc

SEQ ID NO: 55 - RNA, 19 nt, Homo sapiens
  source 1..19: mol_type = genomic RNA
gggggaagaaaaggtgggg

SEQ ID NO: 56 - RNA, 18 nt, Homo sapiens
  source 1..18: mol_type = genomic RNA
cagcagtccctccccctg

SEQ ID NO: 57 - RNA, 18 nt, Homo sapiens
  source 1..18: mol_type = genomic RNA
gggtcccggggagggggg

SEQ ID NO: 58 - RNA, 18 nt, Homo sapiens
  source 1..18: mol_type = genomic RNA
gggctcacatcaccccat

SEQ ID NO: 59 - RNA, 17 nt, Homo sapiens
  source 1..17: mol_type = genomic RNA
accccactcctggtacc

SEQ ID NO: 60 - RNA, 19 nt, Homo sapiens
  source 1..19: mol_type = genomic RNA
aatgttttttcctgtttcc

SEQ ID NO: 61 - RNA, 19 nt, Homo sapiens
  source 1..19: mol_type = genomic RNA
ggccttgttcctgtcccca

SEQ ID NO: 62 - RNA, 20 nt, Homo sapiens
  source 1..20: mol_type = genomic RNA
agccccctggccccaaaccc

SEQ ID NO: 63 - RNA, 18 nt, Homo sapiens
  source 1..18: mol_type = genomic RNA
cagccccacagcctcaga

SEQ ID NO: 64 - RNA, 21 nt, Homo sapiens
  source 1..21: mol_type = genomic RNA
acaggagtgggggtgggacat

SEQ ID NO: 65 - RNA, 21 nt, Homo sapiens
  source 1..21: mol_type = genomic RNA
cgtcccaccccccactcctgt

SEQ ID NO: 66 - RNA, 21 nt, Homo sapiens
  source 1..21: mol_type = genomic RNA
atgtcccacccccactcctgt

SEQ ID NO: 67 - RNA, 17 nt, Homo sapiens
  source 1..17: mol_type = genomic RNA
ggtgggggctgttgttt

SEQ ID NO: 68 - RNA, 22 nt, Homo sapiens
  source 1..22: mol_type = genomic RNA
cagccacaactaccctgccact

SEQ ID NO: 69 - RNA, 22 nt, Homo sapiens
  source 1..22: mol_type = genomic RNA
atggcatcgtcccctggtggct

SEQ ID NO: 70 - RNA, 21 nt, Homo sapiens
  source 1..21: mol_type = genomic RNA
tctgaggcctgcctctcccca

SEQ ID NO: 71 - RNA, 22 nt, Homo sapiens
  source 1..22: mol_type = genomic RNA
gttctgttaacccatcccctca

SEQ ID NO: 72 - RNA, 22 nt, Homo sapiens
  source 1..22: mol_type = genomic RNA
cttccggtctgtgagccccgtc

SEQ ID NO: 73 - RNA, 26 nt, Homo sapiens
  source 1..26: mol_type = genomic RNA
ctcggccgcggcgcgtagcccccgcc

SEQ ID NO: 74 - RNA, 22 nt, Homo sapiens
  source 1..22: mol_type = genomic RNA
actggggagcagaaggagaacc

SEQ ID NO: 75 - RNA, 21 nt, Homo sapiens
  source 1..21: mol_type = genomic RNA
tggctgttggagggggcaggc

SEQ ID NO: 76 - RNA, 22 nt, Homo sapiens
  source 1..22: mol_type = genomic RNA
ttgaggagacatggtgggggcc

SEQ ID NO: 77 - RNA, 24 nt, Homo sapiens
  source 1..24: mol_type = genomic RNA
tgtcagtgactcctgccccttggt

SEQ ID NO: 78 - RNA, 23 nt, Homo sapiens
  source 1..23: mol_type = genomic RNA
ttgaagaggaggtgctctgtagc

SEQ ID NO: 79 - RNA, 22 nt, Homo sapiens
  source 1..22: mol_type = genomic RNA
aactctgaccccttaggttgat

SEQ ID NO: 80 - RNA, 22 nt, Homo sapiens
  source 1..22: mol_type = genomic RNA
tccatgtttccttcccccttct

SEQ ID NO: 81 - RNA, 21 nt, Homo sapiens
  source 1..21: mol_type = genomic RNA
acacatgggtggctgtggcct

SEQ ID NO: 82 - RNA, 25 nt, Homo sapiens
  source 1..25: mol_type = genomic RNA
catgctgacctccctcctgccccag

SEQ ID NO: 83 - RNA, 22 nt, Homo sapiens
  source 1..22: mol_type = genomic RNA
cacacaagtggcccccaacact

SEQ ID NO: 84 - RNA, 20 nt, Homo sapiens
  source 1..20: mol_type = genomic RNA
cgcccctcctgcccccacag

SEQ ID NO: 85 - RNA, 22 nt, Homo sapiens
  source 1..22: mol_type = genomic RNA
cctgacccaccccctcccgcag

SEQ ID NO: 86 - RNA, 23 nt, Homo sapiens
  source 1..23: mol_type = genomic RNA
cagggaggcgctcactctctgct

SEQ ID NO: 87 - RNA, 22 nt, Homo sapiens
  source 1..22: mol_type = genomic RNA
ttacggaccagctaagggaggc

SEQ ID NO: 88 - RNA, 22 nt, Homo sapiens
  source 1..22: mol_type = genomic RNA
tcaggctcagtcccctcccgat

SEQ ID NO: 89 - RNA, 22 nt, Homo sapiens
  source 1..22: mol_type = genomic RNA
tcctgtactgagctgccccgag

SEQ ID NO: 90 - RNA, 22 nt, Homo sapiens
  source 1..22: mol_type = genomic RNA
ttttgtgtctcccattccccag

SEQ ID NO: 91 - RNA, 22 nt, Homo sapiens
  source 1..22: mol_type = genomic RNA
ttctcaagagggaggcaatcat

SEQ ID NO: 92 - RNA, 22 nt, Homo sapiens
  source 1..22: mol_type = genomic RNA
caaagcgctcccctttagaggt

SEQ ID NO: 93 - RNA, 21 nt, Homo sapiens
  source 1..21: mol_type = genomic RNA
atccagttctctgagggggct

SEQ ID NO: 94 - RNA, 22 nt, Homo sapiens
  source 1..22: mol_type = genomic RNA
tgccccaacaaggaaggacaag

SEQ ID NO: 95 - RNA, 20 nt, Homo sapiens
  source 1..20: mol_type = genomic RNA
gcggagagagaatggggagc

SEQ ID NO: 96 - RNA, 21 nt, Homo sapiens
  source 1..21: mol_type = genomic RNA
gggctagggcctgctgccccc

SEQ ID NO: 97 - RNA, 22 nt, Homo sapiens
  source 1..22: mol_type = genomic RNA
gactatagaactttccccctca

SEQ ID NO: 98 - RNA, 22 nt, Homo sapiens
  source 1..22: mol_type = genomic RNA
aaccagcaccccaactttggac

SEQ ID NO: 99 - RNA, 24 nt, Homo sapiens
  source 1..24: mol_type = genomic RNA
actgggggctttcgggctctgcgt

SEQ ID NO: 100 - RNA, 23 nt, Homo sapiens
  source 1..23: mol_type = genomic RNA
aggtctgcattcaaatccccaga

SEQ ID NO: 101 - RNA, 22 nt, Homo sapiens
  source 1..22: mol_type = genomic RNA
caaagtccttcctatttttccc

SEQ ID NO: 102 - RNA, 21 nt, Homo sapiens
  source 1..21: mol_type = genomic RNA
tctctgctctgctctccccag

SEQ ID NO: 103 - RNA, 21 nt, Homo sapiens
  source 1..21: mol_type = genomic RNA
tctattccccactctccccag

SEQ ID NO: 104 - RNA, 20 nt, Homo sapiens
  source 1..20: mol_type = genomic RNA
acctgggttgtcccctctag

SEQ ID NO: 105 - RNA, 19 nt, Homo sapiens
  source 1..19: mol_type = genomic RNA
tggatgacagtggaggcct

SEQ ID NO: 106 - RNA, 21 nt, Homo sapiens
  source 1..21: mol_type = genomic RNA
tccctgcccccatactcccag

SEQ ID NO: 107 - RNA, 20 nt, Homo sapiens
  source 1..20: mol_type = genomic RNA
tccccttcctccctgcccag

SEQ ID NO: 108 - RNA, 22 nt, Homo sapiens
  source 1..22: mol_type = genomic RNA
aacactggccttgctatcccca

SEQ ID NO: 109 - RNA, 22 nt, Homo sapiens
  source 1..22: mol_type = genomic RNA
tagggatgggaggccaggatga

SEQ ID NO: 110 - RNA, 21 nt, Homo sapiens
  source 1..21: mol_type = genomic RNA
acactgtccccttctccccag

SEQ ID NO: 111 - RNA, 21 nt, Homo sapiens
  source 1..21: mol_type = genomic RNA
ccctctctgtcccacccatag

SEQ ID NO: 112 - RNA, 21 nt, Homo sapiens
  source 1..21: mol_type = genomic RNA
aggccctgtcctctgccccag

SEQ ID NO: 113 - RNA, 23 nt, Homo sapiens
  source 1..23: mol_type = genomic RNA
caaccaccactgtctctccccag

SEQ ID NO: 114 - RNA, 20 nt, Homo sapiens
  source 1..20: mol_type = genomic RNA
tccactctcctggcccccag

SEQ ID NO: 115 - RNA, 23 nt, Homo sapiens
  source 1..23: mol_type = genomic RNA
cacctttgtgtccccatcctgca

SEQ ID NO: 116 - RNA, 22 nt, Homo sapiens
  source 1..22: mol_type = genomic RNA
tctcaccccaactctgccccag

SEQ ID NO: 117 - RNA, 21 nt, Homo sapiens
  source 1..21: mol_type = genomic RNA
acatcgccccaccttccccag

SEQ ID NO: 118 - RNA, 21 nt, Homo sapiens
  source 1..21: mol_type = genomic RNA
cgacctcggcgacccctcact

SEQ ID NO: 119 - RNA, 21 nt, Homo sapiens
  source 1..21: mol_type = genomic RNA
acccctcgtttcttcccccag

SEQ ID NO: 120 - RNA, 22 nt, Homo sapiens
  source 1..22: mol_type = genomic RNA
tgcatgacccttccctccccac

SEQ ID NO: 121 - RNA, 23 nt, Homo sapiens
  source 1..23: mol_type = genomic RNA
tgccctgcatggtgtccccacag

SEQ ID NO: 122 - RNA, 21 nt, Homo sapiens
  source 1..21: mol_type = genomic RNA
cacctctcctggcatcgcccc

SEQ ID NO: 123 - RNA, 20 nt, Homo sapiens
  source 1..20: mol_type = genomic RNA
acccctgccactcactggcc

SEQ ID NO: 124 - RNA, 22 nt, Homo sapiens
  source 1..22: mol_type = genomic RNA
ttcacccctctcacctaagcag

SEQ ID NO: 125 - RNA, 20 nt, Homo sapiens
  source 1..20: mol_type = genomic RNA
ctaggtggggggcttgaagc

SEQ ID NO: 126 - RNA, 22 nt, Homo sapiens
  source 1..22: mol_type = genomic RNA
ctgggggtggggggctgggcgt

SEQ ID NO: 127 - RNA, 23 nt, Homo sapiens
  source 1..23: mol_type = genomic RNA
tcccctgctcccttgttccccag

SEQ ID NO: 128 - RNA, 23 nt, Homo sapiens
  source 1..23: mol_type = genomic RNA
atggggacagggatcagcatggc

SEQ ID NO: 129 - RNA, 21 nt, Homo sapiens
  source 1..21: mol_type = genomic RNA
ccgctcttcccctgaccccag

SEQ ID NO: 130 - RNA, 21 nt, Homo sapiens
  source 1..21: mol_type = genomic RNA
aaccttggcccctctccccag

SEQ ID NO: 131 - RNA, 21 nt, Homo sapiens
  source 1..21: mol_type = genomic RNA
aagcctctgtccccaccccag

SEQ ID NO: 132 - RNA, 22 nt, Homo sapiens
  source 1..22: mol_type = genomic RNA
ttggggtggagggccaaggagc

SEQ ID NO: 133 - RNA, 22 nt, Homo sapiens
  source 1..22: mol_type = genomic RNA
tgtgacttctcccctgccacag

SEQ ID NO: 134 - RNA, 21 nt, Homo sapiens
  source 1..21: mol_type = genomic RNA
tctctggtcttgccaccccag

SEQ ID NO: 135 - RNA, 21 nt, Homo sapiens
  source 1..21: mol_type = genomic RNA
accgtctcttctgttccccag

SEQ ID NO: 136 - RNA, 24 nt, Homo sapiens
  source 1..24: mol_type = genomic RNA
acccccgggcaaagacctgcagat

SEQ ID NO: 137 - RNA, 21 nt, Homo sapiens
  source 1..21: mol_type = genomic RNA
accttgcatctgcatccccag

SEQ ID NO: 138 - RNA, 21 nt, Homo sapiens
  source 1..21: mol_type = genomic RNA
tgaccccttctgtctccctag

SEQ ID NO: 139 - RNA, 22 nt, Homo sapiens
  source 1..22: mol_type = genomic RNA
tgggggctggatggggtagagt

SEQ ID NO: 140 - RNA, 19 nt, Homo sapiens
  source 1..19: mol_type = genomic RNA
gtggtctcttggcccccag

SEQ ID NO: 141 - RNA, 22 nt, Homo sapiens
  source 1..22: mol_type = genomic RNA
agactgaccttcaaccccacag

SEQ ID NO: 142 - RNA, 21 nt, Homo sapiens
  source 1..21: mol_type = genomic RNA
tgactgagcttctccccacag

SEQ ID NO: 143 - RNA, 18 nt, Homo sapiens
  source 1..18: mol_type = genomic RNA
tggacctctcctccccag

SEQ ID NO: 144 - RNA, 22 nt, Homo sapiens
  source 1..22: mol_type = genomic RNA
cgggcatgctgggagagacttt

SEQ ID NO: 145 - RNA, 22 nt, Homo sapiens
  source 1..22: mol_type = genomic RNA
gctcatccccatctcctttcag

SEQ ID NO: 146 - RNA, 21 nt, Homo sapiens
  source 1..21: mol_type = genomic RNA
cccatgcctcctgccgcggtc

SEQ ID NO: 147 - RNA, 23 nt, Homo sapiens
  source 1..23: mol_type = genomic RNA
agggagaaagctagaagctgaag

SEQ ID NO: 148 - RNA, 21 nt, Homo sapiens
  source 1..21: mol_type = genomic RNA
ccgccttctctcctcccccag

SEQ ID NO: 149 - RNA, 23 nt, Homo sapiens
  source 1..23: mol_type = genomic RNA
cccatcacctttccgtctcccct

SEQ ID NO: 150 - RNA, 21 nt, Homo sapiens
  source 1..21: mol_type = genomic RNA
ctttgcttcctgctcccctag

SEQ ID NO: 151 - RNA, 21 nt, Homo sapiens
  source 1..21: mol_type = genomic RNA
tcccctccactttcctcctag

SEQ ID NO: 152 - RNA, 20 nt, Homo sapiens
  source 1..20: mol_type = genomic RNA
tctgtgcccctacttcccag

SEQ ID NO: 153 - RNA, 21 nt, Homo sapiens
  source 1..21: mol_type = genomic RNA
tccctctcccaccccttgcag

SEQ ID NO: 154 - RNA, 21 nt, Homo sapiens
  source 1..21: mol_type = genomic RNA
tgacccacccctctccaccag

SEQ ID NO: 155 - RNA, 18 nt, Homo sapiens
  source 1..18: mol_type = genomic RNA
ctggcagggggagaggta

SEQ ID NO: 156 - RNA, 23 nt, Homo sapiens
  source 1..23: mol_type = genomic RNA
tctgctcataccccatggtttct

SEQ ID NO: 157 - RNA, 23 nt, Homo sapiens
  source 1..23: mol_type = genomic RNA
gaagacttcttggattacagggg

SEQ ID NO: 158 - RNA, 23 nt, Homo sapiens
  source 1..23: mol_type = genomic RNA
cggccccacgcaccagggtaaga

SEQ ID NO: 159 - RNA, 20 nt, Homo sapiens
  source 1..20: mol_type = genomic RNA
ggggagctgtggaagcagta

SEQ ID NO: 160 - RNA, 22 nt, Homo sapiens
  source 1..22: mol_type = genomic RNA
ctatacaacttactactttccc

SEQ ID NO: 161 - RNA, 23 nt, Homo sapiens
  source 1..23: mol_type = genomic RNA
tgggcgcgccgggactgtgagac

SEQ ID NO: 162 - RNA, 19 nt, Homo sapiens
  source 1..19: mol_type = genomic RNA
ggcggggctcggagccggg

SEQ ID NO: 163 - RNA, 20 nt, Homo sapiens
  source 1..20: mol_type = genomic RNA
cggcggggctcggagccggg

SEQ ID NO: 164 - RNA, 21 nt, Homo sapiens
  source 1..21: mol_type = genomic RNA
cggcggcggcggctctgggcg

SEQ ID NO: 165 - RNA, 20 nt, Homo sapiens
  source 1..20: mol_type = genomic RNA
caaggaggagcggggattag

SEQ ID NO: 166 - RNA, 26 nt, Homo sapiens
  source 1..26: mol_type = genomic RNA
taaggaacgcggggccttggtagagc

SEQ ID NO: 167 - RNA, 21 nt, Homo sapiens
  source 1..21: mol_type = genomic RNA
ctgccacgagcgtgcgggcct

SEQ ID NO: 168 - RNA, 21 nt, Homo sapiens
  source 1..21: mol_type = genomic RNA
gtgggcgggggcaggtgtgtg

SEQ ID NO: 169 - RNA, 20 nt, Homo sapiens
  source 1..20: mol_type = genomic RNA
gtgtctgggcggacagctgc

SEQ ID NO: 170 - RNA, 21 nt, Homo sapiens
  source 1..21: mol_type = genomic RNA
cgggggcggggccgaagcgcg

SEQ ID NO: 171 - RNA, 18 nt, Homo sapiens
  source 1..18: mol_type = genomic RNA
cgggcgtggtggtggggg

SEQ ID NO: 172 - RNA, 20 nt, Homo sapiens
  source 1..20: mol_type = genomic RNA
cgggcgtggtggtgggggtg

SEQ ID NO: 173 - RNA, 23 nt, Homo sapiens
  source 1..23: mol_type = genomic RNA
cggggccgtagcactgtctgaga

SEQ ID NO: 174 - RNA, 22 nt, Homo sapiens
  source 1..22: mol_type = genomic RNA
ctcggcgcggggcgcgggctcc

SEQ ID NO: 175 - RNA, 21 nt, Homo sapiens
  source 1..21: mol_type = genomic RNA
cggcggggacggcgattggtc

SEQ ID NO: 176 - RNA, 22 nt, Homo sapiens
  source 1..22: mol_type = genomic RNA
cgcaggggccgggtgctcaccg

SEQ ID NO: 177 - RNA, 20 nt, Homo sapiens
  source 1..20: mol_type = genomic RNA
ccccagggcgacgcggcggg

SEQ ID NO: 178 - RNA, 17 nt, Homo sapiens
  source 1..17: mol_type = genomic RNA
ggggcgcggccggatcg

SEQ ID NO: 179 - RNA, 22 nt, Homo sapiens
  source 1..22: mol_type = genomic RNA
tggggcggagcttccggaggcc

SEQ ID NO: 180 - RNA, 17 nt, Homo sapiens
  source 1..17: mol_type = genomic RNA
cgcgccgggcccgggtt

SEQ ID NO: 181 - RNA, 18 nt, Homo sapiens
  source 1..18: mol_type = genomic RNA
cggggcggcaggggcctc

SEQ ID NO: 182 - RNA, 22 nt, Homo sapiens
  source 1..22: mol_type = genomic RNA
gtgggctgggctgggctgggcc

SEQ ID NO: 183 - RNA, 20 nt, Homo sapiens
  source 1..20: mol_type = genomic RNA
cgcgggtcggggtctgcagg

SEQ ID NO: 184 - RNA, 23 nt, Homo sapiens
  source 1..23: mol_type = genomic RNA
tgagcaccacacaggccgggcgc

SEQ ID NO: 185 - RNA, 18 nt, Homo sapiens
  source 1..18: mol_type = genomic RNA
agcaggtgcggggcggcg

SEQ ID NO: 186 - RNA, 20 nt, Homo sapiens
  source 1..20: mol_type = genomic RNA
gtgggttggggcgggctctg

SEQ ID NO: 187 - RNA, 17 nt, Homo sapiens
  source 1..17: mol_type = genomic RNA
ttggaggcgtgggtttt

SEQ ID NO: 188 - RNA, 22 nt, Homo sapiens
  source 1..22: mol_type = genomic RNA
cagggctggcagtgacatgggt

SEQ ID NO: 189 - RNA, 17 nt, Homo sapiens
  source 1..17: mol_type = genomic RNA
ggggctgggcgcgcgcc

SEQ ID NO: 190 - RNA, 17 nt, Homo sapiens
  source 1..17: mol_type = genomic RNA
ctccgggacggctgggc

SEQ ID NO: 191 - RNA, 18 nt, Homo sapiens
  source 1..18: mol_type = genomic RNA
aggctgggctgggacgga

SEQ ID NO: 192 - RNA, 17 nt, Homo sapiens
  source 1..17: mol_type = genomic RNA
gcggggctgggcgcgcg

SEQ ID NO: 193 - RNA, 17 nt, Homo sapiens
  source 1..17: mol_type = genomic RNA
gggagaagggtcggggc

SEQ ID NO: 194 - RNA, 18 nt, Homo sapiens
  source 1..18: mol_type = genomic RNA
cccagcaggacgggagcg

SEQ ID NO: 195 - RNA, 19 nt, Homo sapiens
  source 1..19: mol_type = genomic RNA
cggcgcgaccggcccgggg

SEQ ID NO: 196 - RNA, 22 nt, Homo sapiens
  source 1..22: mol_type = genomic RNA
caccggggatggcagagggtcg

SEQ ID NO: 197 - RNA, 21 nt, Homo sapiens
  source 1..21: mol_type = genomic RNA
ctgggctcgggacgcgcggct

SEQ ID NO: 198 - RNA, 22 nt, Homo sapiens
  source 1..22: mol_type = genomic RNA
taggggcagcagaggacctggg

SEQ ID NO: 199 - RNA, 23 nt, Homo sapiens
  source 1..23: mol_type = genomic RNA
gccccggcgcgggcgggttctgg

SEQ ID NO: 200 - RNA, 23 nt, Homo sapiens
  source 1..23: mol_type = genomic RNA
ggcaggagggctgtgccaggttg

SEQ ID NO: 201 - RNA, 23 nt, Homo sapiens
  source 1..23: mol_type = genomic RNA
ctggcggagcccattccatgcca

SEQ ID NO: 202 - RNA, 22 nt, Homo sapiens
  source 1..22: mol_type = genomic RNA
gctgcgggctgcggtcagggcg

SEQ ID NO: 203 - RNA, 22 nt, Homo sapiens
  source 1..22: mol_type = genomic RNA
ctcgggcggaggtggttgagtg

SEQ ID NO: 204 - RNA, 24 nt, Homo sapiens
  source 1..24: mol_type = genomic RNA
aggcaggggctggtgctgggcggg

SEQ ID NO: 205 - RNA, 22 nt, Homo sapiens
  source 1..22: mol_type = genomic RNA
gcgggggtggcggcggcatccc

SEQ ID NO: 206 - RNA, 21 nt, Homo sapiens
  source 1..21: mol_type = genomic RNA
cggggcagctcagtacaggat

SEQ ID NO: 207 - RNA, 23 nt, Homo sapiens
  source 1..23: mol_type = genomic RNA
ccggggcagattggtgtagggtg

SEQ ID NO: 208 - RNA, 19 nt, Homo sapiens
  source 1..19: mol_type = genomic RNA
gagccagttggacaggagc

SEQ ID NO: 209 - RNA, 20 nt, Homo sapiens
  source 1..20: mol_type = genomic RNA
gggctggggcgcggggaggt

SEQ ID NO: 210 - RNA, 21 nt, Homo sapiens
  source 1..21: mol_type = genomic RNA
acggcccaggcggcattggtg

SEQ ID NO: 211 - RNA, 20 nt, Homo sapiens
  source 1..20: mol_type = genomic RNA
gcggaaggcggagcggcgga

SEQ ID NO: 212 - RNA, 18 nt, Homo sapiens
  source 1..18: mol_type = genomic RNA
gtgaaggcccggcggaga

SEQ ID NO: 213 - RNA, 25 nt, Homo sapiens
  source 1..25: mol_type = genomic RNA
agggatcgcgggcgggtggcggcct

SEQ ID NO: 214 - RNA, 22 nt, Homo sapiens
  source 1..22: mol_type = genomic RNA
aggcggggcgccgcgggaccgc

SEQ ID NO: 215 - RNA, 23 nt, Homo sapiens
  source 1..23: mol_type = genomic RNA
tgggcaggggcttattgtaggag

SEQ ID NO: 216 - RNA, 23 nt, Homo sapiens
  source 1..23: mol_type = genomic RNA
ctgggcccgcggcgggcgtgggg

SEQ ID NO: 217 - RNA, 22 nt, Homo sapiens
  source 1..22: mol_type = genomic RNA
tgggcgagggcggctgagcggc

SEQ ID NO: 218 - RNA, 22 nt, Homo sapiens
  source 1..22: mol_type = genomic RNA
aaggggcagggacgggtggccc

SEQ ID NO: 219 - RNA, 23 nt, Homo sapiens
  source 1..23: mol_type = genomic RNA
cggggccatggagcagcctgtgt

SEQ ID NO: 220 - RNA, 23 nt, Homo sapiens
  source 1..23: mol_type = genomic RNA
cacacaggaaaagcggggccctg

SEQ ID NO: 221 - RNA, 21 nt, Homo sapiens
  source 1..21: mol_type = genomic RNA
cgggccggaggtcaagggcgt

SEQ ID NO: 222 - RNA, 20 nt, Homo sapiens
  source 1..20: mol_type = genomic RNA
gccggggctttgggtgaggg

SEQ ID NO: 223 - RNA, 22 nt, Homo sapiens
  source 1..22: mol_type = genomic RNA
tggcgggggtagagctggctgc

SEQ ID NO: 224 - RNA, 24 nt, Homo sapiens
  source 1..24: mol_type = genomic RNA
gtaggggcgtcccgggcgcgcggg

SEQ ID NO: 225 - RNA, 22 nt, Homo sapiens
  source 1..22: mol_type = genomic RNA
cccctggggctgggcaggcgga

SEQ ID NO: 226 - RNA, 23 nt, Homo sapiens
  source 1..23: mol_type = genomic RNA
ccagggggatgggcgagcttggg

SEQ ID NO: 227 - RNA, 21 nt, Homo sapiens
  source 1..21: mol_type = genomic RNA
gtaggtgacagtcaggggcgg

SEQ ID NO: 228 - RNA, 22 nt, Homo sapiens
  source 1..22: mol_type = genomic RNA
tagggggcggcttgtggagtgt

SEQ ID NO: 229 - RNA, 21 nt, Homo sapiens
  source 1..21: mol_type = genomic RNA
tggggcggggcaggtccctgc

SEQ ID NO: 230 - RNA, 23 nt, Homo sapiens
  source 1..23: mol_type = genomic RNA
gtgcgtggtggctcgaggcgggg

SEQ ID NO: 231 - RNA, 19 nt, Homo sapiens
  source 1..19: mol_type = genomic RNA
cggggccagagcagagagc

SEQ ID NO: 232 - RNA, 22 nt, Homo sapiens
  source 1..22: mol_type = genomic RNA
gtgcggaacgctggccggggcg

SEQ ID NO: 233 - RNA, 22 nt, Homo sapiens
  source 1..22: mol_type = genomic RNA
gtgagtagtggcgcgcggcggc

SEQ ID NO: 234 - RNA, 21 nt, Homo sapiens
  source 1..21: mol_type = genomic RNA
gtgtggccggcaggcgggtgg

SEQ ID NO: 235 - RNA, 22 nt, Homo sapiens
  source 1..22: mol_type = genomic RNA
tgcggggctagggctaacagca

SEQ ID NO: 236 - RNA, 22 nt, Homo sapiens
  source 1..22: mol_type = genomic RNA
ggggctggggccggggccgagc

SEQ ID NO: 237 - RNA, 19 nt, Homo sapiens
  source 1..19: mol_type = genomic RNA
cggggtcggcggcgacgtg

SEQ ID NO: 238 - RNA, 22 nt, Homo sapiens
  source 1..22: mol_type = genomic RNA
tcaaaatcaggagtcggggctt

SEQ ID NO: 239 - RNA, 23 nt, Homo sapiens
  source 1..23: mol_type = genomic RNA
ggatggttgggggcggtcggcgt

SEQ ID NO: 240 - RNA, 20 nt, Homo sapiens
  source 1..20: mol_type = genomic RNA
ggcggcggggaggtaggcag

SEQ ID NO: 241 - RNA, 22 nt, Homo sapiens
  source 1..22: mol_type = genomic RNA
gtgaacgggcgccatcccgagg

SEQ ID NO: 242 - RNA, 22 nt, Homo sapiens
  source 1..22: mol_type = genomic RNA
agggacgggacgcggtgcagtg

SEQ ID NO: 243 - RNA, 87 nt, Homo sapiens
  source 1..87: mol_type = genomic RNA
accgcagggaaaatgagggacttttgggggcagatgtgtttccattccactatcataatgcccctaaaaatccttattgctcttgca

SEQ ID NO: 244 - RNA, 111 nt, Homo sapiens
  source 1..111: mol_type = genomic RNA
agagtgttcaaggacagcaagaaaaatgagggactttcaggggcagctgtgttttctgactcagtcataatgcccctaaaaatccttattgttcttgcagtgtgcatcggg

SEQ ID NO: 245 - RNA, 83 nt, Homo sapiens
  source 1..83: mol_type = genomic RNA
cggggtgaggtagtaggttgtgtggtttcagggcagtgatgttgcccctcggaagataactatacaacctactgccttccctg

SEQ ID NO: 246 - RNA, 87 nt, Homo sapiens
  source 1..87: mol_type = genomic RNA
tcagagtgaggtagtagattgtatagttgtggggtagtgattttaccctgttcaggagataactatacaatctattgccttccctga

SEQ ID NO: 247 - RNA, 97 nt, Homo sapiens
  source 1..97: mol_type = genomic RNA
gggacttgtcactgcctgtctcctccctctccagcagcgactggattctggagtccatctagagggtcttgggagggatgtgactgttgggaagccc

SEQ ID NO: 248 - RNA, 86 nt, Homo sapiens
  source 1..86: mol_type = genomic RNA
tttggtacttgaagagaggataccctttgtatgttcacttgattaatggcgaatatacagggggagactcttatttgcgtatcaaa

SEQ ID NO: 249 - RNA, 86 nt, Homo sapiens
  source 1..86: mol_type = genomic RNA
tttggtacttaaagagaggataccctttgtatgttcacttgattaatggcgaatatacagggggagactctcatttgcgtatcaaa

SEQ ID NO: 250 - RNA, 87 nt, Homo sapiens
  source 1..87: mol_type = genomic RNA
gcagggctggcagggaggctgggaggggctggctgggtctggtagtgggcatcagctggccctcatttcttaagacagcacttctgt

SEQ ID NO: 251 - RNA, 85 nt, Homo sapiens
  source 1..85: mol_type = genomic RNA
gtgaggactcgggaggtggagggtggtgccgccggggccgggcgctgtttcagctcgcttctccccccacctcctctctcctcag

SEQ ID NO: 252 - RNA, 90 nt, Homo sapiens
  source 1..90: mol_type = genomic RNA
gtgggtacggcccagtgggggggagagggacacgccctgggctctgcccagggtgcagccggactgactgagcccctgtgccgcccccag

SEQ ID NO: 253 - RNA, 90 nt, Homo sapiens; identical to SEQ ID NO: 252
  source 1..90: mol_type = genomic RNA

SEQ ID NO: 254 - RNA, 88 nt, Homo sapiens
  source 1..88: mol_type = genomic RNA
gtggggccaggcggtggtgggcactgctggggtgggcacagcagccatgcagagcgggcatttgaccccgtgccacccttttccccag

SEQ ID NO: 255 - RNA, 73 nt, Homo sapiens
  source 1..73: mol_type = genomic RNA
gtgggcgggggcaggtgtgtggtgggtggtggcctgcggtgagcagggccctcacacctgcctcgccccccag

SEQ ID NO: 256 - RNA, 82 nt, Homo sapiens
  source 1..82: mol_type = genomic RNA
gtgagtgggaggccagggcacggcagggggagctgcagggctatgggaggggccccagcgtctgagccctgtcctcccgcag

SEQ ID NO: 257 - RNA, 80 nt, Homo sapiens
  source 1..80: mol_type = genomic RNA
gtgagtgtggggtggctggggcggggggggcccggggacggcttgggcctgcctagtcggcctgaccacccaccccacag

SEQ ID NO: 258 - RNA, 102 nt, Homo sapiens
  source 1..102: mol_type = genomic RNA
gtgggagggcccaggcgcgggcaggggtgggggtggcagagcgctgtcccgggggcggggccgaagcgcggcgaccgtaactccttctgctccgtcccccag

SEQ ID NO: 259 - RNA, 83 nt, Homo sapiens
  source 1..83: mol_type = genomic RNA
gtgagtgggagccccagtgtgtggttggggccatggcgggtgggcagcccagcctctgagccttcctcgtctgtctgccccag

SEQ ID NO: 260 - RNA, 136 nt, Homo sapiens
  source 1..136: mol_type = genomic RNA
ccgcttgcctcgcccagcgcagccccggccgctgggcgcacccgtcccgttcgtccccggacgttgctctctaccccgggaacgtcgagactggagcgcccgaactgagccaccttcgcggaccccgagagcggcg

SEQ ID NO: 261 - RNA, 86 nt, Homo sapiens
  source 1..86: mol_type = genomic RNA
tgccagtctctaggtccctgagaccctttaacctgtgaggacatccagggtcacaggtgaggttcttgggagcctggcgtctggcc

SEQ ID NO: 262 - RNA, 78 nt, Homo sapiens
  source 1..78: mol_type = genomic RNA
ctcccaaatctcctgttgaagtgtaatccccacctccagcattggggattacatttcaacatgagatttggatgagga

SEQ ID NO: 263 - RNA, 80 nt, Homo sapiens
  source 1..80: mol_type = genomic RNA
cctctgtgagaaagggtgtgggggagaggctgtcttgtgtctgtaagtatgccaaacttattttccccaaggcagaggga

SEQ ID NO: 264 - RNA, 72 nt, Homo sapiens
  source 1..72: mol_type = genomic RNA
ggatctttttgcggtctgggcttgctgttcctctcaacagtagtcaggaagcccttaccccaaaaagtatct

SEQ ID NO: 265 - RNA, 90 nt, Homo sapiens
  source 1..90: mol_type = genomic RNA
tgcccttcgcgaatctttttgcggtctgggcttgctgtacataactcaatagccggaagcccttaccccaaaaagcatttgcggagggcg

SEQ ID NO: 266 - RNA, 91 nt, Homo sapiens
  source 1..91: mol_type = genomic RNA
aaacacttgagcccagcggtttgaggctacagtgagatgtgatcctgccacatctcactgtagcctcgaacccctgggctcaagtgattca

SEQ ID NO: 267 - RNA, 73 nt, Homo sapiens
  source 1..73: mol_type = genomic RNA
actgaggtcctcaaaactgaggggcattttctgtggtttgaaaggaaagtgcacccagttttggggatgtcaa

SEQ ID NO: 268 - RNA, 88 nt, Homo sapiens
  source 1..88: mol_type = genomic RNA
acaatgctttgctagagctggtaaaatggaaccaaatcgcctcttcaatggatttggtccccttcaaccagctgtagctatgcattga

SEQ ID NO: 269 - RNA, 119 nt, Homo sapiens
  source 1..119: mol_type = genomic RNA
cctcagaagaaagatgccccctgctctggctggtcaaacggaaccaagtccgtcttcctgagaggtttggtccccttcaaccagctacagcagggctggcaatgcccagtccttggaga

SEQ ID NO: 270 - RNA, 73 nt, Homo sapiens
  source 1..73: mol_type = genomic RNA
cagggtgtgtgactggttgaccagaggggcatgcactgtgttcaccctgtgggccacctagtcaccaaccctc

SEQ ID NO: 271 - RNA, 71 nt, Homo sapiens
  source 1..71: mol_type = genomic RNA
tgtgttaaggtgcatctagtgcagttagtgaagcagcttagaatctactgccctaaatgccccttctggca

SEQ ID NO: 272 - RNA, 92 nt, Homo sapiens
  source 1..92: mol_type = genomic RNA
cggctggacagcgggcaacggaatcccaaaagcagctgttgtctccagagcattccagctgcgcttggatttcgtcccctgctctcctgcct

SEQ ID NO: 273 - RNA, 71 nt, Homo sapiens
  source 1..71: mol_type = genomic RNA
gccaacccagtgttcagactacctgttcaggaggctctcaatgtgtacagtagtctgcacattggttaggc

SEQ ID NO: 274 - RNA, 110 nt, Homo sapiens
  source 1..110: mol_type = genomic RNA
ccagaggacacctccactccgtctacccagtgtttagactatctgttcaggactcccaaattgtacagtagtctgcacattggttaggctgggctgggttagaccctcgg

SEQ ID NO: 275 - RNA, 110 nt, Homo sapiens
  source 1..110: mol_type = genomic RNA
acccggcagtgcctccaggcgcagggcagcccctgcccaccgcacactgcgctgccccagacccactgtgcgtgtgacagcggctgatctgtgcctgggcagcgcgaccc

SEQ ID NO: 276 - RNA, 80 nt, Homo sapiens
  source 1..80: mol_type = genomic RNA
gacctaggctaggggttcttagcataggaggtcttcccatgctaagaagtcctcccatgccaagaactcccagactagga

SEQ ID NO: 277 - RNA, 82 nt, Homo sapiens
  source 1..82: mol_type = genomic RNA
gcagactggaaaatctctgcaggcaaatgtgatgtcactgaggaaatcacacacttacccgtagagattctacagtctgaca

SEQ ID NO: 278 - RNA, 110 nt, Homo sapiens
  source 1..110: mol_type = genomic RNA
cctggcctcctgcagtgccacgctccgtgtatttgacaagctgagttggacactccatgtggtagagtgtcagtttgtcaaataccccaagtgcggcacatgcttaccag

SEQ ID NO: 279 - RNA, 80 nt, Homo sapiens
  source 1..80: mol_type = genomic RNA
aggacccttccagagggccccccctcaatcctgttgtgcctaattcagagggttgggtggaggctctcctgaagggctct

SEQ ID NO: 280 - RNA, 85 nt, Homo sapiens
  source 1..85: mol_type = genomic RNA
ccctactctgggaaggtgccattctgagggccaggagtttgattatgtgtcactctggctgctatggccccctcccagggtctgg

SEQ ID NO: 281 - RNA, 86 nt, Homo sapiens
  source 1..86: mol_type = genomic RNA
gagggaaagcaggccaacctcgaggatctccccagccttggcgttcaggtgctgaggagatcgtcgaggttggcctgcttcccctc

SEQ ID NO: 282 - RNA, 82 nt, Homo sapiens
  source 1..82: mol_type = genomic RNA
ctgacttttttagggagtagaagggtggggagcatgaacaatgtttctcactccctacccctccactccccaaaaaagtcag

SEQ ID NO: 283 - RNA, 73 nt, Homo sapiens
  source 1..73: mol_type = genomic RNA
gcctcagttgccccatctgtgccctgggtaggaatatcctggatccccttgggtctgatggggtagccgatgc

SEQ ID NO: 284 - RNA, 80 nt, Homo sapiens
  source 1..80: mol_type = genomic RNA
ctggggtcacctgtctggccagctacgtccccacggcccttgtcagtgtggaaggtagacggccagagaggtgaccccgg

SEQ ID NO: 285 - RNA, 75 nt, Homo sapiens
  source 1..75: mol_type = genomic RNA
tggagtgggggggcaggaggggctcagggagaaagtgcatacagcccctggccctctctgcccttccgtcccctg

SEQ ID NO: 286 - RNA, 94 nt, Homo sapiens
  source 1..94: mol_type = genomic RNA
gagtttggttttgtttgggtttgttctaggtatggtcccagggatcccagatcaaaccaggcccctgggcctatcctagaaccaacctaagctc

SEQ ID NO: 287 - RNA, 72 nt, Homo sapiens
  source 1..72: mol_type = genomic RNA
ggagcttatcagaatctccaggggtactttataatttcaaaaagtcccccaggtgtgattctgatttgcttc

SEQ ID NO: 288 - RNA, 86 nt, Homo sapiens
  source 1..86: mol_type = genomic RNA
ggttctgtcttgggccacttggatctgaaggctgcccctttgctctctggggtagccttcagatcttggtgttttgaattcttact

SEQ ID NO: 289 - RNA, 87 nt, Homo sapiens; identical to SEQ ID NO: 243
  source 1..87: mol_type = genomic RNA

SEQ ID NO: 290 - RNA, 65 nt, Homo sapiens
  source 1..65: mol_type = genomic RNA
gaaggcagcagtgctcccctgtgacgtgctccatcaccgggcagggaagacaccgctgccacctc

SEQ ID NO: 291 - RNA, 67 nt, Homo sapiens
  source 1..67: mol_type = genomic RNA
gtggcactcaaactgtgggggcactttctgctctctggtgaaagtgccgccatcttttgagtgttac

SEQ ID NO: 292 - RNA, 66 nt, Homo sapiens
  source 1..66: mol_type = genomic RNA
ggtaacactcaaaagatggcggcactttcaccagagagcagaaagtgcccccacagtttgagtgcc

SEQ ID NO: 293 - RNA, 64 nt, Homo sapiens
  source 1..64: mol_type = genomic RNA
ccccgcgacgagcccctcgcacaaaccggacctgagcgttttgttcgttcggctcgcgtgaggc

SEQ ID NO: 294 - RNA, 100 nt, Homo sapiens
  source 1..100: mol_type = genomic RNA
cacacagacggcagctgcggcctagcccccaggcttcacttggcgtggacaacttgctaagtaaagtggggggtgggccacggctggctcctacctggac

SEQ ID NO: 295 - RNA, 79 nt, Homo sapiens
  source 1..79: mol_type = genomic RNA
tggtactcggggagaggttacccgagcaactttgcatctggacgacgaatgttgctcggtgaaccccttttcggtatca

SEQ ID NO: 296 - RNA, 84 nt, Homo sapiens
  source 1..84: mol_type = genomic RNA
acagcgccctgcaggcacagacagccctggcttctgcctctttctttgtggaagccactctgtcaggcctgggatggaggggca

SEQ ID NO: 297 - RNA, 67 nt, Homo sapiens
  source 1..67: mol_type = genomic RNA
aaatctctctccatatctttcctgcagcccccaggtgggggggaagaaaaggtggggaattagattc

SEQ ID NO: 298 - RNA, 91 nt, Homo sapiens
  source 1..91: mol_type = genomic RNA
ggggcatttagggtaactgagctgctgccggggcctggcgctcctctaccttgtcaggtgacccagcagtccctccccctgcatggtgccc

SEQ ID NO: 299 - RNA, 62 nt, Homo sapiens
  source 1..62: mol_type = genomic RNA
gctgggggtcccccgacagtgtggagctggggccgggtcccggggaggggggttctgggcag

SEQ ID NO: 300 - RNA, 81 nt, Homo sapiens
  source 1..81: mol_type = genomic RNA
gttctgtgaggggctcacatcaccccatcaaagtggggactcatggggagagggggtagttaggagctttgatagaggcgg

SEQ ID NO: 301 - RNA, 93 nt, Homo sapiens
  source 1..93: mol_type = genomic RNA
tacttatggcaccccactcctggtaccatagtcataagttaggagatgttagagctgtgagtaccatgacttaagtgtggtggcttaaacatg

SEQ ID NO: 302 - RNA, 84 nt, Homo sapiens
  source 1..84: mol_type = genomic RNA
tcagaagaaaaaacaggagataaagtttgtgataatgtttgtctatatagttatgaatgttttttcctgtttccttcagggcca

SEQ ID NO: 303 - RNA, 76 nt, Homo sapiens
  source 1..76: mol_type = genomic RNA
gaaaggttgggggcacagagagcaaggagccttccccagaggagtcaggccttgttcctgtccccattcctcagag

SEQ ID NO: 304 - RNA, 101 nt, Homo sapiens
  source 1..101: mol_type = genomic RNA
gatcaggcccagccccctggccccaaaccctgcagccccagctggaggatgaggagatgctgggcttgggtgggggaatcaggggtgtaaaggggcctgct

SEQ ID NO: 305 - RNA, 69 nt, Homo sapiens
  source 1..69: mol_type = genomic RNA
cggggcccaggcgggcatgtggggtgtctggagacgccaggcagccccacagcctcagacctcgggcac

SEQ ID NO: 306 - RNA, 81 nt, Homo sapiens
  source 1..81: mol_type = genomic RNA
catcctccttacgtcccaccccccactcctgtttctggtgaaatattcaaacaggagtgggggtgggacataaggaggata

SEQ ID NO: 307 - RNA, 81 nt, Homo sapiens; identical to SEQ ID NO: 306
  source 1..81: mol_type = genomic RNA

SEQ ID NO: 308 - RNA, 102 nt, Homo sapiens
  source 1..102: mol_type = genomic RNA
tgtgttccctatcctccttatgtcccacccccactcctgtttgaatatttcaccagaaacaggagtggggggtgggacgtaaggaggatgggggaaagaaca

SEQ ID NO: 309 - RNA, 91 nt, Homo sapiens
  source 1..91: mol_type = genomic RNA
gttctagagcatggtttctcatcatttgcactactgatacttggggtcagataattgtttgtggtgggggctgttgtttgcattgtaggat

SEQ ID NO: 310 - RNA, 97 nt, Homo sapiens
  source 1..97: mol_type = genomic RNA
tgacctgaatcaggtaggcagtgtattgttagctggctgcttgggtcaagtcagcagccacaactaccctgccacttgcttctggataaattcttct

SEQ ID NO: 311 - RNA, 82 nt, Homo sapiens
  source 1..82: mol_type = genomic RNA
cacaactgcatggcatcgtcccctggtggctgtggcctagggcaagccacaaagccactcagtgatgatgccagcagttgtg

SEQ ID NO: 312 - RNA, 64 nt, Homo sapiens
  source 1..64: mol_type = genomic RNA
tctgggcgaggggtgggctctcagaggggctggcagtactgctctgaggcctgcctctccccag

SEQ ID NO: 313 - RNA, 78 nt, Homo sapiens
  source 1..78: mol_type = genomic RNA
tattggacgaggggactggttaatagaactaactaaccagaactattttgttctgttaacccatcccctcatctaata

SEQ ID NO: 314 - RNA, 71 nt, Homo sapiens
  source 1..71: mol_type = genomic RNA
gttgggggctggggtgcccactccgcaagttatcactgagcgacttccggtctgtgagccccgtcctccgc

SEQ ID NO: 315 - RNA, 79 nt, Homo sapiens
  source 1..79: mol_type = genomic RNA
ctcgaggtgctgggggacgcgtgagcgcgagccgcttcctcacggctcggccgcggcgcgtagcccccgccacatcggg

SEQ ID NO: 316 - RNA, 66 nt, Homo sapiens
  source 1..66: mol_type = genomic RNA
tgactggggagcagaaggagaacccaagaaaagctgacttggaggtccctccttctgtccccacag

SEQ ID NO: 317 - RNA, 80 nt, Homo sapiens
  source 1..80: mol_type = genomic RNA
acctgaggagccagccctcctcccgcacccaaacttggagcacttgacctttggctgttggagggggcaggctcgcgggt

SEQ ID NO: 318 - RNA, 70 nt, Homo sapiens
  source 1..70: mol_type = genomic RNA
ggtttctccttgaggagacatggtgggggccggtcaggcagcccatgccatgtgtcctcatggagaggcc

SEQ ID NO: 319 - RNA, 78 nt, Homo sapiens
  source 1..78: mol_type = genomic RNA
gggcccagaagggggcgcagtcactgacgtgaagggaccacatcccgcttcatgtcagtgactcctgccccttggtct

SEQ ID NO: 320 - RNA, 72 nt, Homo sapiens
  source 1..72: mol_type = genomic RNA
ctgcttcaacaacagtgacttgctctccaatggtatccagtgattcgttgaagaggaggtgctctgtagcag

SEQ ID NO: 321 - RNA, 77 nt, Homo sapiens
  source 1..77: mol_type = genomic RNA
attttggccaactctgaccccttaggttgatgtcagaatgaggtgtaccaacctaggtggtcagagttggccaaaat

SEQ ID NO: 322 - RNA, 84 nt, Homo sapiens
  source 1..84: mol_type = genomic RNA
catactttgtctccatgtttccttcccccttctgtatacatgtatacaggaggaagggggaaggaaacatggagacaaagtgtg

SEQ ID NO: 323 - RNA, 72 nt, Homo sapiens
  source 1..72: mol_type = genomic RNA
ggcagtgtttaggccacagccacccatgtgtaggggtggctacacatgggtggctgtggcctaaacactgcc

SEQ ID NO: 324 - RNA, 67 nt, Homo sapiens
  source 1..67: mol_type = genomic RNA
gtgggaggggagaggcagcaagcacacagggcctgggactagcatgctgacctccctcctgccccag

SEQ ID NO: 325 - RNA, 70 nt, Homo sapiens
  source 1..70: mol_type = genomic RNA
ccctgccagtgctgggggccacatgagtgtgcagtcatccacacacaagtggcccccaacactggcaggg

SEQ ID NO: 326 - RNA, 61 nt, Homo sapiens
  source 1..61: mol_type = genomic RNA
cctgcggggacaggccagggcatctaggctgtgcacagtgacgcccctcctgcccccacag

SEQ ID NO: 327 - RNA, 56 nt, Homo sapiens
  source 1..56: mol_type = genomic RNA
cgctcgggcggaggtggttgagtgccgactggcgcctgacccaccccctcccgcag

SEQ ID NO: 328 - RNA, 78 nt, Homo sapiens
  source 1..78: mol_type = genomic RNA
gggataaaatgcagggaggcgctcactctctgctgccgattctgcaccagagatggttgccttcctatattttgtgtc

SEQ ID NO: 329 - RNA, 80 nt, Homo sapiens
  source 1..80: mol_type = genomic RNA
aatgaaggattacggaccagctaagggaggcattaggatccttattcttgcctcccttagttggtccctaatccttcgtt

SEQ ID NO: 330 - RNA, 79 nt, Homo sapiens
  source 1..79: mol_type = genomic RNA
agcctcgtcaggctcagtcccctcccgataaacccctaaatagggactttcccggggggtgaccctggcttttttggcg

SEQ ID NO: 331 - RNA, 68 nt, Homo sapiens
  source 1..68: mol_type = genomic RNA
gcatcctgtactgagctgccccgaggcccttcatgctgcccagctcggggcagctcagtacaggatac

SEQ ID NO: 332 - RNA, 120 nt, Homo sapiens
  source 1..120: mol_type = genomic RNA
gatccagggaaccctagagcagggggatggcagagcaaaattcatggcctacagctgcctcttgccaaactgcactggattttgtgtctcccattccccagagctgtctgaggtgctttg

SEQ ID NO: 333 - RNA, 80 nt, Homo sapiens
  source 1..80: mol_type = genomic RNA
catgtggtactcttctcaagagggaggcaatcatgtgtaattagatatgattgacacctctgtgagtggagtaacacatg

SEQ ID NO: 334 - RNA, 83 nt, Homo sapiens
  source 1..83: mol_type = genomic RNA
tcatgctgtggccctccagagggaagcgctttctgttgtctgaaagaaaacaaagcgctcccctttagaggtttacggtttga

SEQ ID NO: 335 - RNA, 115 nt, Homo sapiens
  source 1..115: mol_type = genomic RNA
gagcaaaaaccagagaacaacatgggagcgttcctaacccctaaggcaactggatgggagacctgacccatccagttctctgagggggctcttgtgtgttctacaaggttgttca

SEQ ID NO: 336 - RNA, 90 nt, Homo sapiens
  source 1..90: mol_type = genomic RNA
ctgtacccctgccccaacaaggaaggacaagaggtgtgagccacacacacgcctggcctcctgtctttccttgttggagcagggatgtag

SEQ ID NO: 337 - RNA, 80 nt, Homo sapiens
  source 1..80: mol_type = genomic RNA
ggttggctataactatcatttccaaggttgtgcttttaggaaatgttggctgtcctgcggagagagaatggggagccagg

SEQ ID NO: 338 - RNA, 79 nt, Homo sapiens
  source 1..79: mol_type = genomic RNA
tggtgacccctgggctagggcctgctgccccctgcccagtgcaggagggtggagggtcactccttaggtggtcccagtg

SEQ ID NO: 339 - RNA, 85 nt, Homo sapiens
  source 1..85: mol_type = genomic RNA
agggtagagggatgagggggaaagttctatagtcctgtaattagatctcaggactatagaactttccccctcatccctctgccct

SEQ ID NO: 340 - RNA, 97 nt, Homo sapiens
  source 1..97: mol_type = genomic RNA
aaacccacaccactgcattttggccatcgagggttggggcttggtgtcatgccccaagataaccagcaccccaactttggacagcatggattagtct

SEQ ID NO: 341 - RNA, 99 nt, Homo sapiens
  source 1..99: mol_type = genomic RNA
tggctaaggtgttggctcgggctccccactgcagttaccctcccctcggcgttactgagcactgggggctttcgggctctgcgtctgcacagatacttc

SEQ ID NO: 342 - RNA, 86 nt, Homo sapiens
  source 1..86: mol_type = genomic RNA
aatggtccccccagggaggtctgcattcaaatccccagaagctgaggattaggggactaggatgcagacctccctgggggaccatt

SEQ ID NO: 343 - RNA, 70 nt, Homo sapiens
  source 1..70: mol_type = genomic RNA
ggagggaagaataggagggactttgtattgtggttcagtaccatgcaaagtccttcctatttttccctcc

SEQ ID NO: 344 - RNA, 89 nt, Homo sapiens
  source 1..89: mol_type = genomic RNA
ctagattgggatggtaggaccagaggggcttactgccctgtggggctctctggacccagtgccatgcttctctgctctgctctccccag

SEQ ID NO: 345 - RNA, 72 nt, Homo sapiens
  source 1..72: mol_type = genomic RNA
acaggtgggagagcagggtattgtggaagctccaggtgccaaccacctgcctctattccccactctccccag

SEQ ID NO: 346 - RNA, 62 nt, Homo sapiens
  source 1..62: mol_type = genomic RNA
gagggagtggggtgggacccagctgttggccatggcgacaacacctgggttgtcccctctag

SEQ ID NO: 347 - RNA, 66 nt, Homo sapiens
  source 1..66: mol_type = genomic RNA
tcacgtggatgacagtggaggcctcctggatctctaggtctcagggcctctcttgtcatcctgcag

SEQ ID NO: 348 - RNA, 71 nt, Homo sapiens
  source 1..71: mol_type = genomic RNA
atggaggggggtgtggagccagggggcccaggtctacagcttctccccgctccctgcccccatactcccag

SEQ ID NO: 349 - RNA, 63 nt, Homo sapiens
  source 1..63: mol_type = genomic RNA
accctagggtggggctggaggtggggctgaggctgagtcttcctccccttcctccctgcccag

SEQ ID NO: 350 - RNA, 69 nt, Homo sapiens
  source 1..69: mol_type = genomic RNA
gggcttagggatgggaggccaggatgaagattaatccctaatccccaacactggccttgctatccccag

SEQ ID NO: 351 - RNA, 69 nt, Homo sapiens; identical to SEQ ID NO: 350
  source 1..69: mol_type = genomic RNA

SEQ ID NO: 352 - RNA, 68 nt, Homo sapiens
  source 1..68: mol_type = genomic RNA
cagtgcagggagaaggtggaagtgcagagtgggctcacctctcgcccacactgtccccttctccccag

SEQ ID NO: 353 - RNA, 62 nt, Homo sapiens
  source 1..62: mol_type = genomic RNA
cttcctggtgggtggggaggagaagtgccgtcctcatgagcccctctctgtcccacccatag

SEQ ID NO: 354 - RNA, 69 nt, Homo sapiens
  source 1..69: mol_type = genomic RNA
gaacctcggggcatgggggagggaggctggacaggagagggctcacccaggccctgtcctctgccccag

SEQ ID NO: 355 - RNA, 59 nt, Homo sapiens
  source 1..59: mol_type = genomic RNA
cgggctctgggtgcagtgggggttcccacgccgcggcaaccaccactgtctctccccag

SEQ ID NO: 356 - RNA, 66 nt, Homo sapiens
  source 1..66: mol_type = genomic RNA
tcaagacggggagtcaggcagtggtggagatggagagccctgagcctccactctcctggcccccag

SEQ ID NO: 357 - RNA, 69 nt, Homo sapiens
  source 1..69: mol_type = genomic RNA
tggggtaggggtgggggaattcaggggtgtcgaactcatggctgccacctttgtgtccccatcctgcag

SEQ ID NO: 358 - RNA, 67 nt, Homo sapiens
  source 1..67: mol_type = genomic RNA
tacaggccggggctttgggtgagggacccccggagtctgtcacggtctcaccccaactctgccccag

SEQ ID NO: 359 - RNA, 81 nt, Homo sapiens
  source 1..81: mol_type = genomic RNA
ctccctgggagggcgtggatgatggtgggagaggagccccactgtggaagtctgacccccacatcgccccaccttccccag

SEQ ID NO: 360 - RNA, 63 nt, Homo sapiens
  source 1..63: mol_type = genomic RNA
gtgagtgtggatttggcggggttcgggggttccgacggcgacctcggcgacccctcactcacc

SEQ ID NO: 361 - RNA, 68 nt, Homo sapiens
  source 1..68: mol_type = genomic RNA
agggttggggggacaggatgagaggctgtcttcattccctcttgaccacccctcgtttcttcccccag

SEQ ID NO: 362 - RNA, 72 nt, Homo sapiens
  source 1..72: mol_type = genomic RNA
cagccaggagggaaggggctgagaacaggacctgtgctcactggggcctgcatgacccttccctccccacag

SEQ ID NO: 363 - RNA, 69 nt, Homo sapiens
  source 1..69: mol_type = genomic RNA
gaggaggggaggtgtgcagggctggggtcactgactctgcttcccctgccctgcatggtgtccccacag

SEQ ID NO: 364 - RNA, 82 nt, Homo sapiens
  source 1..82: mol_type = genomic RNA
acctgtaggtgacagtcaggggcggggtgtggtggggctggggctggccccctcctcacacctctcctggcatcgcccccag

SEQ ID NO: 365 - RNA, 79 nt, Homo sapiens
  source 1..79: mol_type = genomic RNA
tggcctggtcagaggcagcaggaaatgagagttagccaggagctttgcatactcacccctgccactcactggcccccag

SEQ ID NO: 366 - RNA, 65 nt, Homo sapiens
  source 1..65: mol_type = genomic RNA
gagggctaggtggggggcttgaagccccgagatgcctcacgtcttcacccctctcacctaagcag

SEQ ID NO: 367 - RNA, 65 nt, Homo sapiens; identical to SEQ ID NO: 366
  source 1..65: mol_type = genomic RNA

SEQ ID NO: 368 - RNA, 65 nt, Homo sapiens
  source 1..65: mol_type = genomic RNA
ctcctctgggggtggggggctgggcgtggtggacagcgatgcatccctcgccttctcaccctcag

SEQ ID NO: 369 - RNA, 70 nt, Homo sapiens
  source 1..70: mol_type = genomic RNA
ctgggatggggacagggatcagcatggcacagatccaataccttctgtcccctgctcccttgttccccag

SEQ ID NO: 370 - RNA, 70 nt, Homo sapiens; identical to SEQ ID NO: 369
  source 1..70: mol_type = genomic RNA

SEQ ID NO: 371 - RNA, 64 nt, Homo sapiens
  source 1..64: mol_type = genomic RNA
tgaggatggggtgagatggggaggagcagccagtcctgtctcaccgctcttcccctgaccccag

SEQ ID NO: 372 - RNA, 56 nt, Homo sapiens
  source 1..56: mol_type = genomic RNA
gtaggcaggggctggggtttcaggttctcagtcagaaccttggcccctctccccag

SEQ ID NO: 373 - RNA, 61 nt, Homo sapiens
  source 1..61: mol_type = genomic RNA
gagggttggggtggagggccaaggagctgggtggggtgccaagcctctgtccccaccccag

SEQ ID NO: 374 - RNA, 61 nt, Homo sapiens; identical to SEQ ID NO: 373
  source 1..61: mol_type = genomic RNA

SEQ ID NO: 375 - RNA, 62 nt, Homo sapiens
  source 1..62: mol_type = genomic RNA
ccttctgcggcagagctggggtcaccagccctcatgtacttgtgacttctcccctgccacag

SEQ ID NO: 376 - RNA, 63 nt, Homo sapiens
  source 1..63: mol_type = genomic RNA
gaggtgtaggggaggttgggccagggatgccttcactgtgtctctctggtcttgccaccccag

SEQ ID NO: 377 - RNA, 59 nt, Homo sapiens
  source 1..59: mol_type = genomic RNA
tctggtgggagccatgagggtctgtgctgtctctgagcaccgtctcttctgttccccag

SEQ ID NO: 378 - RNA, 71 nt, Homo sapiens
  source 1..71: mol_type = genomic RNA
tgaccacccccgggcaaagacctgcagatcccctgttagagacgggcccaggactttgtgcggggtgccca

SEQ ID NO: 379 - RNA, 72 nt, Homo sapiens
  source 1..72: mol_type = genomic RNA
gtgtttagggtactcagagcaagttgtgaaacacaggtgttttttaacctcaccttgcatctgcatccccag

SEQ ID NO: 380 - RNA, 60 nt, Homo sapiens
  source 1..60: mol_type = genomic RNA
caggctgggggctggatggggtagagtaggagagcccactgaccccttctgtctccctag

SEQ ID NO: 381 - RNA, 60 nt, Homo sapiens; identical to SEQ ID NO: 380
  source 1..60: mol_type = genomic RNA

SEQ ID NO: 382 - RNA, 70 nt, Homo sapiens
  source 1..70: mol_type = genomic RNA
gtccctgggggctgggatgggccatggtgtgctctgatccccctgtggtctcttggcccccaggaactcc

SEQ ID NO: 383 - RNA, 67 nt, Homo sapiens
  source 1..67: mol_type = genomic RNA
gctgcttggggtttggggtgcagacattgccagaggatgggcagcagactgaccttcaaccccacag

SEQ ID NO: 384 - RNA, 93 nt, Homo sapiens
  source 1..93: mol_type = genomic RNA
gcttgttggggattgggtcaggccagtgttcaagggcccctcctctagtactccctgtttgtgttctgccactgactgagcttctccccacag

SEQ ID NO: 385 - RNA, 64 nt, Homo sapiens
  source 1..64: mol_type = genomic RNA
gaggcactgggtaggtggggctccagggctcctgacacctggacctctcctccccaggcccaca

SEQ ID NO: 386 - RNA, 70 nt, Homo sapiens
  source 1..70: mol_type = genomic RNA
cgaagcgggcatgctgggagagactttgtgatttgtctccaaagcctcacccagctctctggccctctag

SEQ ID NO: 387 - RNA, 60 nt, Homo sapiens
  source 1..60: mol_type = genomic RNA
caaggtgggggagatgggggttgaacttcatttctcatgctcatccccatctcctttcag

SEQ ID NO: 388 - RNA, 62 nt, Homo sapiens
  source 1..62: mol_type = genomic RNA
gtgggtctcgcatcaggaggcaaggccaggacccgctgacccatgcctcctgccgcggtcag

SEQ ID NO: 389 - RNA, 66 nt, Homo sapiens
  source 1..66: mol_type = genomic RNA
atgagagggagaaagctagaagctgaagattctgaaaatcactaactggcctcttctttctcctag

SEQ ID NO: 390 - RNA, 62 nt, Homo sapiens
  source 1..62: mol_type = genomic RNA
gagggtggtggaggaagagggcagctcccatgactgcctgaccgccttctctcctcccccag

SEQ ID NO: 391 - RNA, 78 nt, Homo sapiens
  source 1..78: mol_type = genomic RNA
cccgcagaggctgagaaggtgatgttggctcaagaaagggagatagatggtagcccatcacctttccgtctcccctag

SEQ ID NO: 392 - RNA, 66 nt, Homo sapiens
  source 1..66: mol_type = genomic RNA
cctggaggggggcactgcgcaagcaaagccagggaccctgagaggctttgcttcctgctcccctag

SEQ ID NO: 393 - RNA, 65 nt, Homo sapiens
  source 1..65: mol_type = genomic RNA
gagaatggggggacagatggagaggacacaggctggcactgaggtcccctccactttcctcctag

SEQ ID NO: 394 - RNA, 59 nt, Homo sapiens
  source 1..59: mol_type = genomic RNA
ctgtgtcggggagtctggggtccggaattctccagagcctctgtgcccctacttcccag

SEQ ID NO: 395 - RNA, 115 nt, Homo sapiens
  source 1..115: mol_type = genomic RNA
gtaagggaccggagagtaggaaaagcagggctcagggccagagagactgggcatagaactaaggaggatggtgtcctcctgactgcatctctcttccctctcccaccccttgcag

SEQ ID NO: 396 - RNA, 61 nt, Homo sapiens
  source 1..61: mol_type = genomic RNA
tccgctctgtggagtggggtgcctgtcccctgccactgggtgacccacccctctccaccag

SEQ ID NO: 397 - RNA, 94 nt, Homo sapiens
  source 1..94: mol_type = genomic RNA
cacggtgtcccctggtggaacctggcagggggagaggtaaggtctttcagcctctccaaagcccatggtcaggtactcaggtgggggagccctg

SEQ ID NO: 398 - RNA, 109 nt, Homo sapiens
  source 1..109: mol_type = genomic RNA
gcttttatattgtaggtttttgctcatgcaccatggttgtctgagcatgcagcatgcttgtctgctcataccccatggtttctgagcaggaaccttcattgtctactgc

SEQ ID NO: 399 - RNA, 78 nt, Homo sapiens
  source 1..78: mol_type = genomic RNA
tctaagaagtgaagacttcttggattacaggggccctactttaagggccctttcagttggaagttttcctttctgcct

SEQ ID NO: 400 - RNA, 78 nt, Homo sapiens
  source 1..78: mol_type = genomic RNA
ttagccctgcggccccacgcaccagggtaagagagactctcgcttcctgccctggcccgagggaccgactggctgggc

SEQ ID NO: 401 - RNA, 75 nt, Homo sapiens
  source 1..75: mol_type = genomic RNA
gtagttgttctacagaagacctggatgtgtaggagctaagacacactccaggggagctgtggaagcagtaacacg

SEQ ID NO: 402 - RNA, 119 nt, Homo sapiens
  source 1..119: mol_type = genomic RNA
aggattctgctcatgccagggtgaggtagtaagttgtattgttgtggggtagggatattaggccccaattagaagataactatacaacttactactttccctggtgtgtggcatattca

SEQ ID NO: 403 - RNA, 83 nt, Homo sapiens
  source 1..83: mol_type = genomic RNA
tctgcaggtcctggtgaacgccatcatcaacagtggtccccgggaggactccacacgcattgggcgcgccgggactgtgagac

SEQ ID NO: 404 - RNA, 49 nt, Homo sapiens
  source 1..49: mol_type = genomic RNA
ggcggggctcggagccgggcttcggccgggccccgggccctcgaccggg

SEQ ID NO: 405 - RNA, 51 nt, Homo sapiens
  source 1..51: mol_type = genomic RNA
cggcggggctcggagccgggcttcggccgggccccgggccctcgaccggac

SEQ ID NO: 406 - RNA, 55 nt, Homo sapiens
  source 1..55: mol_type = genomic RNA
cggcggcggcggctctgggcgaggcggcggggcctgggctcccggacgaggcggg

SEQ ID NO: 407 - RNA, 88 nt, Homo sapiens
  source 1..88: mol_type = genomic RNA
gggtcaaggaggagcggggattagttctaggggctgtaggagggtgacagtcctggactgaaggtcacctgcttggctctgatgattt

SEQ ID NO: 408 - RNA, 90 nt, Homo sapiens
  source 1..90: mol_type = genomic RNA
ctggctgggcggtaaggaacgcggggccttggtagagcaaagtgcggaccaaagactttgcgtctggttgcttttaccttgcctagtagg

SEQ ID NO: 409 - RNA, 98 nt, Homo sapiens
  source 1..98: mol_type = genomic RNA
tgggctcggcccgggctgccacgagcgtgcgggcctcgccgggcatgtcctaggcggcggccccgcccagcgctcggccgggcgggcgggcgggcgcg

SEQ ID NO: 410 - RNA, 73 nt, Homo sapiens; identical to SEQ ID NO: 255
  source 1..73: mol_type = genomic RNA

SEQ ID NO: 411 - RNA, 92 nt, Homo sapiens
  source 1..92: mol_type = genomic RNA
gtcagtgtctgggcggacagctgcaggaaagggaagaccaaggcttgctgtctgtccagtctgccaccctaccctgtctgttcttgccacag

SEQ ID NO: 412 - RNA, 102 nt, Homo sapiens; identical to SEQ ID NO: 258
  source 1..102: mol_type = genomic RNA

SEQ ID NO: 413 - RNA, 52 nt, Homo sapiens
  source 1..52: mol_type = genomic RNA
tagccgggcgtggtggtgggggcctgtggtcccagctactttggaggctgag

SEQ ID NO: 414 - RNA, 50 nt, Homo sapiens
  source 1..50: mol_type = genomic RNA
acccgggcgtggtggtgggggtgggtgcctgtaattccagctagttggga

SEQ ID NO: 415 - RNA, 82 nt, Homo sapiens
  source 1..82: mol_type = genomic RNA
tgagctgttggattcggggccgtagcactgtctgagaggtttacatttctcacagtgaaccggtctctttttcagctgcttc

SEQ ID NO: 416 - RNA, 47 nt, Homo sapiens
  source 1..47: mol_type = genomic RNA
ctcggcgcggggcgcgggctccgggttggggcgagccaacgccgggg

SEQ ID NO: 417 - RNA, 80 nt, Homo sapiens
  source 1..80: mol_type = genomic RNA
cgggaatgccgcggcggggacggcgattggtccgtatgtgtggtgccaccggccgccggctccgccccggcccccgcccc

SEQ ID NO: 418 - RNA, 80 nt, Homo sapiens
  source 1..80: mol_type = genomic RNA
catccaggacaatggtgagtgccggtgcctgccctggggccgtccctgcgcaggggccgggtgctcaccgcatctgcccc

SEQ ID NO: 419 - RNA, 80 nt, Homo sapiens
  source 1..80: mol_type = genomic RNA
tgagaggccgcaccttgccttgctgcccgggccgtgcacccgtgggccccagggcgacgcggcgggggcggccctagcga

SEQ ID NO: 420 - RNA, 84 nt, Homo sapiens
  source 1..84: mol_type = genomic RNA
gaggctgggcggggcgcggccggatcggtcgagagcgtcctggctgatgacggtctcccgtgcccacgccccaaacgcagtctc

SEQ ID NO: 421 - RNA, 94 nt, Homo sapiens
  source 1..94: mol_type = genomic RNA
cagtgcgacgggcggagcttccagacgctccgccccacgtcgcatgcgccccgggaaagcgtggggcggagcttccggaggccccgccctgctg

SEQ ID NO: 422 - RNA, 84 nt, Homo sapiens
  source 1..84: mol_type = genomic RNA
ccgcagccgccgcgccgggcccgggttggccgctgacccccgcggggcccccggcggccggggcgggggcgggggctgccccgg

SEQ ID NO: 423 - RNA, 64 nt, Homo sapiens
  source 1..64: mol_type = genomic RNA
gggtgggggcggggcggcaggggcctcccccagtgccaggccccattctgcttctctcccagct

SEQ ID NO: 424 - RNA, 79 nt, Homo sapiens
  source 1..79: mol_type = genomic RNA
gtgaggtgggggccagcagggagtgggctgggctgggctgggccaaggtacaaggcctcaccctgcatcccgcacccag

SEQ ID NO: 425 - RNA, 85 nt, Homo sapiens
  source 1..85: mol_type = genomic RNA
gtgagctgctggggacgcgggtcggggtctgcagggcggtgcggcagccgccacctgacgccgcgcctttgtctgtgtcccacag

SEQ ID NO: 426 - RNA, 97 nt, Homo sapiens
  source 1..97: mol_type = genomic RNA
cccgggaccttggtccaggcgctggtctgcgtggtgctcgggtggataagtctgatctgagcaccacacaggccgggcgccgggaccaagggggctc

SEQ ID NO: 427 - RNA, 105 nt, Homo sapiens
  source 1..105: mol_type = genomic RNA
gcgggcggcggcggcggcagcagcagcaggtgcggggcggcggccgcgctggccgctcgactccgcagctgctcgttctgcttctccagcttgcgcaccagctcc

SEQ ID NO: 428 - RNA, 102 nt, Homo sapiens
  source 1..102: mol_type = genomic RNA
gcttatcgaggaaaagatcgaggtgggttggggcgggctctggggatttggtctcacagcccggatcccagcccacttaccttggttactctccttccttct

SEQ ID NO: 429 - RNA, 53 nt, Homo sapiens
  source 1..53: mol_type = genomic RNA
ggtgggggttggaggcgtgggttttagaacctatccctttctagccctgagca

SEQ ID NO: 430 - RNA, 67 nt, Homo sapiens
  source 1..67: mol_type = genomic RNA
ctggtccatttccctgccattcccttggcttcaatttactcccagggctggcagtgacatgggtcaa

SEQ ID NO: 431 - RNA, 80 nt, Homo sapiens
  source 1..80: mol_type = genomic RNA
ctgcagcgtgcttctccaggccccgcgcgcggacagacacacggacaagtcccgccaggggctgggcgcgcgccagccgg

SEQ ID NO: 432 - RNA, 89 nt, Homo sapiens
  source 1..89: mol_type = genomic RNA
acctccgggacggctgggcgccggcggccgggagatccgcgcttcctgaatcccggccggcccgcccggcgcccgtccgcccgcgggtc

SEQ ID NO: 433 - RNA, 73 nt, Homo sapiens
  source 1..73: mol_type = genomic RNA
ggaggctgggctgggacggacacccggcctccactttctgtggcaggtacctcctccatgtcggcccgccttg

SEQ ID NO: 434 - RNA, 70 nt, Homo sapiens
  source 1..70: mol_type = genomic RNA
aggacccagcggggctgggcgcgcggagcagcgctgggtgcagcgcctgcgccggcagctgcaagggccg

SEQ ID NO: 435 - RNA, 86 nt, Homo sapiens
  source 1..86: mol_type = genomic RNA
agggagaagggtcggggcagggagggcagggcaggctctggggtggggggtctgtgagtcagccacggctctgcccacgtctcccc

SEQ ID NO: 436 - RNA, 56 nt, Homo sapiens
  source 1..56: mol_type = genomic RNA
cgaccgcacccgcccgaagctgggtcaaggagcccagcaggacgggagcgcggcgc

SEQ ID NO: 437 - RNA, 54 nt, Homo sapiens
  source 1..54: mol_type = genomic RNA
ggacaagggcggcgcgaccggcccggggctcttgggcggccgcgtttcccctcc

SEQ ID NO: 438 - RNA, 74 nt, Homo sapiens
  source 1..74: mol_type = genomic RNA
ccaagggcacaccggggatggcagagggtcgtgggaaagtgttgaccctcgtcaggtccccggggagcccctgg

SEQ ID NO: 439 - RNA, 87 nt, Homo sapiens
  source 1..87: mol_type = genomic RNA
cccaggcgcccgctcccgacccacgccgcgccgccgggtccctcctccccggagaggctgggctcgggacgcgcggctcagctcggg

SEQ ID NO: 440 - RNA, 83 nt, Homo sapiens
  source 1..83: mol_type = genomic RNA
gtctactcccagggtgccaagctgtttcgtgttccctccctaggggatcccaggtaggggcagcagaggacctgggcctggac

SEQ ID NO: 441 - RNA, 80 nt, Homo sapiens
  source 1..80: mol_type = genomic RNA
ggttccggagccccggcgcgggcgggttctggggtgtagacgctgctggccagcccgccccagccgaggttctcggcacc

SEQ ID NO: 442 - RNA, 60 nt, Homo sapiens
  source 1..60: mol_type = genomic RNA
ggcaggagggctgtgccaggttggctgggccaggcctgacctgccagcacctccctgcag

SEQ ID NO: 443 - RNA, 76 nt, Homo sapiens
  source 1..76: mol_type = genomic RNA
cgcaggcctctggcggagcccattccatgccagatgctgagcgatggctggtgtgtgctgctccacaggcctggtg

SEQ ID NO: 444 - RNA, 70 nt, Homo sapiens
  source 1..70: mol_type = genomic RNA
ctcgggcccgaccgcgccggcccgcacctcccggcccggagctgcgggctgcggtcagggcgatcccggg

SEQ ID NO: 445 - RNA, 56 nt, Homo sapiens; identical to SEQ ID NO: 327
  source 1..56: mol_type = genomic RNA

SEQ ID NO: 446 - RNA, 92 nt, Homo sapiens
  source 1..92: mol_type = genomic RNA
cctgtccctcctgccctgcgcctgcccagccctcctgctctggtgactgaggaccgccaggcaggggctggtgctgggcggggggcggcggg

SEQ ID NO: 447 - RNA, 84 nt, Homo sapiens
  source 1..84: mol_type = genomic RNA
cggtccagacgtggcgggggtggcggcggcatcccggacggcctgtgagggatgcgccgcccactgccccgcgccgcctgaccg

SEQ ID NO: 448 - RNA, 68 nt, Homo sapiens; identical to SEQ ID NO: 331
  source 1..68: mol_type = genomic RNA

SEQ ID NO: 449 - RNA, 85 nt, Homo sapiens
  source 1..85: mol_type = genomic RNA
tctgaggtacccggggcagattggtgtagggtgcaaagcctgcccgccccctaagccttctgcccccaactccagcctgtcagga

SEQ ID NO: 450 - RNA, 94 nt, Homo sapiens
  source 1..94: mol_type = genomic RNA
aattcagccctgccactggcttatgtcatgaccttgggctactcaggctgtctgcacaatgagccagttggacaggagcagtgccactcaactc

SEQ ID NO: 451 - RNA, 55 nt, Homo sapiens
  source 1..55: mol_type = genomic RNA
gggggctggggcgcggggaggtgctaggtcggcctcggctcccgcgccgcacccc

SEQ ID NO: 452 - RNA, 95 nt, Homo sapiens
  source 1..95: mol_type = genomic RNA
gacaccacatgctcctccaggcctgcctgccctccaggtcatgttccagtgtcccacagatgcagcaccacggcccaggcggcattggtgtcacc

SEQ ID NO: 453 - RNA, 96 nt, Homo sapiens
  source 1..96: mol_type = genomic RNA
gctctggggcgtgccgccgccgtcgctgccacctcccctaccgctagtggaagaagatggcggaaggcggagcggcggatctggacacccagcggt

SEQ ID NO: 454 - RNA, 89 nt, Homo sapiens
  source 1..89: mol_type = genomic RNA
agcctgtgggaaagagaagagcagggcagggtgaaggcccggcggagacactctgcccaccccacaccctgcctatgggccacacagct

SEQ ID NO: 455 - RNA, 100 nt, Homo sapiens
  source 1..100: mol_type = genomic RNA
gtgagcgggcgcggcagggatcgcgggcgggtggcggcctagggcgcggagggcggaccgggaatggcgcgccgtgcgccgccggcgtaactgcggcgct

SEQ ID NO: 456 - RNA, 93 nt, Homo sapiens
  source 1..93: mol_type = genomic RNA
ccttccggcgtcccaggcggggcgccgcgggaccgccctcgtgtctgtggcggtgggatcccgcggccgtgttttcctggtggcccggccatg

SEQ ID NO: 457 - RNA, 87 nt, Homo sapiens
  source 1..87: mol_type = genomic RNA
ccctcatctctgggcaggggcttattgtaggagtctctgaagagagctgtggactgacctgctttaacccttccccaggttcccatt

SEQ ID NO: 458 - RNA, 92 nt, Homo sapiens
  source 1..92: mol_type = genomic RNA
cgctgcgcttctgggcccgcggcgggcgtggggctgcccgggccggtcgaccagcgcgccgtagctcccgaggcccgagccgcgacccgcgg

SEQ ID NO: 459 - RNA, 65 nt, Homo sapiens
  source 1..65: mol_type = genomic RNA
gagggtgggcgagggcggctgagcggctccatcccccggcctgctcatccccctcgccctctcag

SEQ ID NO: 460 - RNA, 71 nt, Homo sapiens
  source 1..71: mol_type = genomic RNA
gggtaaaggggcagggacgggtggccccaggaagaagggcctggtggagccgctcttctccctgcccacag

SEQ ID NO: 461 - RNA, 86 nt, Homo sapiens
  source 1..86: mol_type = genomic RNA
agagccggggccatggagcagcctgtgtagacggggacctgccctgcatgggcaccccctcactggctgcttcccttggtctccag

SEQ ID NO: 462 - RNA, 72 nt, Homo sapiens
  source 1..72: mol_type = genomic RNA
ccaggcacacaggaaaagcggggccctgggttcggctgctaccccaaaggccacattctcctgtgcacacag

SEQ ID NO: 463 - RNA, 64 nt, Homo sapiens
  source 1..64: mol_type = genomic RNA
aaccccgggccggaggtcaagggcgtcgcttctccctaatgttgcctcttttccacggcctcag

SEQ ID NO: 464 - RNA, 67 nt, Homo sapiens; identical to SEQ ID NO: 358
  source 1..67: mol_type = genomic RNA

SEQ ID NO: 465 - RNA, 61 nt, Homo sapiens
  source 1..61: mol_type = genomic RNA
tcggctggcgggggtagagctggctgcaggcccggcccctctcagctgctgccctctccag

SEQ ID NO: 466 - RNA, 98 nt, Homo sapiens
  source 1..98: mol_type = genomic RNA
cgaggtaggggcgtcccgggcgcgcgggcgggtcccaggctgggcccctcggaggccgggtgctcactgccccgtcccggcgcccgtgtctcctccag

SEQ ID NO: 467 - RNA, 67 nt, Homo sapiens
  source 1..67: mol_type = genomic RNA
ccagacccctggggctgggcaggcggaaagaggtctgaactgcctctgcctccttggtctccggcag

SEQ ID NO: 468 - RNA, 67 nt, Homo sapiens
  source 1..67: mol_type = genomic RNA
ggcagccagggggatgggcgagcttgggcccattcctttccttaccctaccccccatccccctgtag

SEQ ID NO: 469 - RNA, 82 nt, Homo sapiens; identical to SEQ ID NO: 364
  source 1..82: mol_type = genomic RNA

SEQ ID NO: 470 - RNA, 62 nt, Homo sapiens
  source 1..62: mol_type = genomic RNA
tggcctagggggcggcttgtggagtgtatgggctgagccttgctctgctcccccgcccccag

SEQ ID NO: 471 - RNA, 66 nt, Homo sapiens
  source 1..66: mol_type = genomic RNA
ccgagtggggcggggcaggtccctgcagggactgtgacactgaaggacctgcaccttcgcccacag

SEQ ID NO: 472 - RNA, 74 nt, Homo sapiens
  source 1..74: mol_type = genomic RNA
gtgcgtggtggctcgaggcgggggtgggggcctcgccctgcttgggccctccctgacctctccgctccgcacag

SEQ ID NO: 473 - RNA, 61 nt, Homo sapiens
  source 1..61: mol_type = genomic RNA
aactgcggggccagagcagagagcccttgcacaccaccagcctctcctccctgtgccccag

SEQ ID NO: 474 - RNA, 61 nt, Homo sapiens
  source 1..61: mol_type = genomic RNA
gtgcggaacgctggccggggcgggaggggaagggacgcccggccggaacgccgcactcacg

SEQ ID NO: 475 - RNA, 62 nt, Homo sapiens
  source 1..62: mol_type = genomic RNA
gtgagtagtggcgcgcggcggctcggagtacctctgccgccgcgcgcatcggctcagcatgc

SEQ ID NO: 476 - RNA, 87 nt, Homo sapiens
  source 1..87: mol_type = genomic RNA
gtgtggccggcaggcgggtgggcgggggcggccggtgggaaccccgccccgccccgcgcccgcactcacccgcccgtctccccacag

SEQ ID NO: 477 - RNA, 98 nt, Homo sapiens
  source 1..98: mol_type = genomic RNA
ttgggcaaggtgcggggctagggctaacagcagtcttactgaaggtttcctggaaaccacgcacatgctgttgccactaacctcaaccttactcggtc

SEQ ID NO: 478 - RNA, 83 nt, Homo sapiens
  source 1..83: mol_type = genomic RNA
ggcccggctccgggtctcggcccgtacagtccggccggccatgctggcggggctggggccggggccgagcccgcggcggggcc

SEQ ID NO: 479 - RNA, 59 nt, Homo sapiens
  source 1..59: mol_type = genomic RNA
cggggtcggcggcgacgtgctcagcttggcacccaagttctgccgctccgacgcccggc

SEQ ID NO: 480 - RNA, 81 nt, Homo sapiens
  source 1..81: mol_type = genomic RNA
tagaggcagtttcaacagatgtgtagacttttgatatgagaaattggtttcaaaatcaggagtcggggctttactgctttt

SEQ ID NO: 481 - RNA, 86 nt, Homo sapiens
  source 1..86: mol_type = genomic RNA
cgcctgagcgtgcagcaggacatcttcctgacctggtaataattaggtgagaaggatggttgggggcggtcggcgtaactcaggga

SEQ ID NO: 482 - RNA, 80 nt, Homo sapiens
  source 1..80: mol_type = genomic RNA
gcgtcaagatggcggcggggaggtaggcagagcaggacgccgctgctgccgccgccaccgccgcctccgctccagtcgcc

SEQ ID NO: 483 - RNA, 79 nt, Homo sapiens
  source 1..79: mol_type = genomic RNA
gtgcagatccttgggagccctgttagactctggattttacacttggagtgaacgggcgccatcccgaggctttgcacag

SEQ ID NO: 484 - RNA, 96 nt, Homo sapiens
  source 1..96: mol_type = genomic RNA
cgggccccgggcgggcgggagggacgggacgcggtgcagtgttgttttttcccccgccaatattgcactcgtcccggcctccggcccccccggccc

(19) United States
(12) Patent Application Publication
(10) Pub. No.: US 2024/0068034 A1
(43) Pub. Date: Feb. 29, 2024

(54) MIRNAS, COMPOSITIONS, AND METHODS OF USING THEREOF

(71) Applicant: Craif Inc., Tokyo (JP)

(72) Inventor: Hiroki YAMAGUCHI, Tokyo (JP)

(21) Appl. No.: 18/472,942

(22) Filed: Sep. 22, 2023

Related U.S. Application Data
(63) Continuation-in-part of application No. PCT/JP2022/013311, filed on Mar. 22, 2022.
(60) Provisional application No. 63/165,508, filed on Mar. 24, 2021.

(51) Int. Cl. C12Q 1/6883; B01L 3/00; C12Q 1/6806; C12Q 1/6841; C12Q 1/6874

(52) U.S. Cl. CPC C12Q 1/6883; B01L 3/502715; C12Q 1/6806; C12Q 1/6841; C12Q 1/6874; B82Y 30/00

(57) ABSTRACT

A method for identifying a patient as having a marker correlated with systemic lupus erythematosus (SLE) comprises obtaining a body fluid sample from a patient suspected of having SLE, analyzing miRNA expression in the obtained body fluid sample, and identifying the patient as having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample, or as not having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

### REFERENCE TO SEQUENCE LISTING SUBMITTED AS A COMPLIANT ASCII TEXT FILE (.xml)

Pursuant to the EFS-Web legal framework and 37 CFR §§ 1.821-5 825 (see MPEP § 2442.03(a)), a Sequence Listing in the form of an ASCII-compliant text file (entitled “3000068-016000_Sequence_Listing_ST26.xml” created on 22 Sep. 2023, and 439,451 bytes in size) is submitted concurrently with the instant application, and the entire contents of the Sequence Listing are incorporated herein by reference.

### CROSS-REFERENCE TO RELATED APPLICATIONS

This application is a continuation-in-part of International Application No. PCT/JP2022/013311, filed 22 Mar. 2022, which claims priority to U.S. Provisional Application No. 63/165,508, filed 24 Mar. 2021, the entire content of each is incorporated herein by reference.

### TECHNICAL FIELD

The present disclosure provides methods for diagnosing systemic lupus erythematosus (SLE) and systems for detecting miRNAs associated with SLE.

### DESCRIPTION OF RELATED ART

Systemic lupus erythematosus (SLE) is a chronic autoimmune disorder involving multiple organs and having diverse clinical manifestations. Among the rheumatic diseases, it has one of the highest mortality rates and is the most common form of lupus. Clinical features of SLE range from mild involvement of skin and joints to severe debilitating complications at later stages, such as infections and problems of renal, cardiovascular, and central nervous system, which are responsible for considerable morbidity and mortality. Being an autoimmune disease, SLE is characterized by the presence of antibodies against the self-antigens. The deposition of autoantibodies and immune complexes in the tissues leads to inflammatory damage of various organ systems of the body. CDC Website “Systemic lupus erythematosus (SLE)” (2020). There is a need in the art for rapid and efficient methods for diagnosing SLE.

### BRIEF SUMMARY

In an aspect, the present disclosure relates to methods for identifying a patient as having a marker correlated with systemic lupus erythematosus (SLE) comprising
(a) obtaining a body fluid sample from a patient suspected of having SLE, (b) analyzing miRNA expression in the obtained body fluid sample, and
(c) identifying the patient
(i) as having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample, or
(ii) as not having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

In another aspect, the analyzing may comprise generating an miRNA profile from the body fluid sample, including:
(a) introducing the obtained body fluid sample into a fluidic device comprising a nanowire,
(b) capturing extracellular vesicles in the body fluid sample on the nanowire,
(c) disrupting the captured extracellular vesicles,
(d) extracting miRNAs from the disrupted extracellular vesicles,
(e) hybridizing the extracted miRNA to an miRNA array; and,
(f) determining miRNA hybridization to the array.

In another aspect, methods of the present disclosure may further comprise comparing the miRNA expression in the body fluid sample obtained from a patient suspected of having SLE with that in the body fluid sample obtained from a healthy individual.

In another aspect, the body fluid may be blood, urine, saliva, ascites, bronchoalveolar lavage fluid, plasma, cerebrospinal fluid, or a combination thereof.

In another aspect, the nanowire may be at least one positively charged surface selected from the group consisting of ZnO, SiO₂, Li₂O, MgO, Al₂O₃, CaO, TiO₂, Mn₂O₃, Fe₂O₃, CoO, NiO, CuO, Ga₂O₃, SrO, In₂O₃, SnO₂, Sm₂O₃, EuO, and combinations thereof.

In another aspect, the nanowire may be porous and/or magnetic.

In another aspect, the captured extracellular vesicles may be disrupted by the use of a cytolysis buffer. The extracellular vesicles may be disrupted by alkali/detergent pre-treatment, storage at about −25° C., for about 1-10 days, optionally about 7 days, or a combination thereof.

In another aspect, the extracting miRNAs may be performed in situ.

In another aspect, the analyzing may comprise,
(a) extracting extracellular vesicles from the obtained body fluid sample,
(b) analyzing oligonucleotide sequences of RNA included in the extracted extracellular vesicles,
(c) generating an miRNA profile from the body fluid based on the analyzed sequences.

The said step (a) may be a step using the fluidic device comprising a nanowire mentioned above, ultracentrifugation, density gradient centrifugation, immunoaffinity purification, ultrafiltration, polymer-based precipitation, size-exclusion chromatography, and a combination thereof. The said step (b) may comprise purifying RNA from the extracted extracellular vesicles, preparing a cDNA library of miRNA included in the purified RNA, and analyzing oligonucleotide sequences of the cDNA library.

In an aspect, the present disclosure relates to methods for identifying a patient as having a marker correlated with SLE severity, including:
a) obtaining a body fluid sample from a patient suspected of having SLE,
b) analyzing miRNA expression in the obtained body fluid sample, and
c) identifying the patient
i) as having the marker correlated with moderate SLE if a decrease in expression of at least one miRNA selected from SEQ ID NOs: 161-242 and 403-484 compared to a body fluid sample obtained from a healthy individual is detected in the patient sample, or
ii) as not having the marker correlated with moderate SLE if a decrease in expression of at least one miRNA selected from SEQ ID NOs: 161-242 and 403-484 compared to a body fluid sample obtained from a healthy individual fails to be detected.

In an aspect, the present disclosure relates to methods for identifying a patient as having a marker correlated with a comorbidity of SLE, including:
a) obtaining a body fluid sample from a patient suspected of having SLE,
b) analyzing miRNA expression in the obtained body fluid sample, and
c) identifying the patient i) as having the marker correlated with a comorbidity of SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample, or ii) as not having the marker correlated with a comorbidity of SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

In another aspect, the comorbidity may be A, if an increase in expression of at least one miRNA selected from SEQ ID NOs: 1-160 and 243-402 and/or a decrease in expression of at least one miRNA selected from SEQ ID NOs: 161-242 and 403-484.

In another aspect, the comorbidity may be B, if an increase in expression of at least one miRNA selected from SEQ ID NOs: 1-160 and 243-402 and/or a decrease in expression of at least one miRNA selected from SEQ ID NOs: 161-242 and 403-484.

In another aspect, the comorbidity may be C, if an increase in expression of at least one miRNA selected from SEQ ID NOs: 1-160 and 243-402 and/or a decrease in expression of at least one miRNA selected from SEQ ID NOs: 161-242 and 403-484.

In another aspect, the comorbidity may be D, if an increase in expression of at least one miRNA selected from SEQ ID NOs: 1-160 and 243-402 and/or a decrease in expression of at least one miRNA selected from SEQ ID NOs: 161-242 and 403-484.

In an aspect, the present disclosure relates to methods of treating SLE, including identifying a patient as having a marker correlated with SLE and administering to the patient an effective amount of a compound selected from the group consisting of nonsteroidal anti-inflammatory drugs (NSAIDs), immunosuppressants, and anti-BLyS antibody.

In an aspect, the present disclosure relates to methods of treating SLE, including identifying a patient as having a marker correlated with moderate SLE and administering to the patient an effective amount of a compound selected from the group consisting of nonsteroidal anti-inflammatory drugs (NSAIDs), immunosuppressants, and anti-BLyS antibody.

In an aspect, the present disclosure relates to methods of treating SLE, including identifying a patient as having a marker correlated with SLE comorbidity A and administering to the patient an effective amount of a compound selected from the group consisting of nonsteroidal anti-inflammatory drugs (NSAIDs), immunosuppressants, and anti-BLyS antibody.

In an aspect, the present disclosure relates to methods of treating SLE, including identifying a patient as having a marker correlated with SLE comorbidity B and administering to the patient an effective amount of a compound selected from the group consisting of nonsteroidal anti-inflammatory drugs (NSAIDs), immunosuppressants, and anti-BLyS antibody.

In an aspect, the present disclosure relates to methods of treating SLE, including identifying a patient as having a marker correlated with SLE comorbidity C and administering to the patient an effective amount of a compound selected from the group consisting of nonsteroidal anti-inflammatory drugs (NSAIDs), immunosuppressants, and anti-BLyS antibody.

In an aspect, the present disclosure relates to methods of treating SLE, including identifying a patient as having a marker correlated with SLE comorbidity D and administering to the patient an effective amount of a compound selected from the group consisting of nonsteroidal anti-inflammatory drugs (NSAIDs), immunosuppressants, and anti-BLyS antibody.

The patent or application file contains at least one drawing executed in color. Copies of this patent or patent application publication with color drawing(s) will be provided by the Office upon request and payment of the necessary fee.

The advantages and features of the present invention will become better understood with reference to the following more detailed description taken in conjunction with the accompanying drawings in which:

### BRIEF DESCRIPTION OF DRAWINGS

FIG. 1 depicts an exemplary procedure of miRNA analysis.

FIG. 2 depicts differential expression analysis conducted by comparing each miRNA signals from SLE patients and healthy donors according to one embodiment of the present disclosure. Fold change among cohorts plotted against p-value of t-test for each miRNA, and statistically significant miRNAs (p values<0.05) were selected as biomarker candidates.

FIG. 3A depicts expression levels of top 10 up-regulated miRNAs shown in FIG. 2.

FIG. 3B depicts expression levels of top 10 down-regulated miRNAs shown in FIG. 2.

FIG. 4 depicts correlation of expression levels of each miRNA with degree of SLE severity according to one embodiment of the present disclosure.

Scatter plot of fold changes of each miRNAs indicates x-axis: SLE vs non-SLE, and y-axis: Moderate SLE vs Mild SLE).

FIG. 5A depicts box plot of expression levels of top 10 up-regulated miRNAs in mild SLE patients (Mild), moderate SLE patients (Moderate), and healthy individuals (None).

FIG. 5B depicts box plot of expression levels of top 10 down-regulated miRNAs in mild SLE patients (Mild), moderate SLE patients (Moderate), and healthy individuals (None).

FIG. 6 depicts comparison of expression levels of miRNAs in SLE patients with or without comorbidity A according to one embodiment of the present disclosure. miRNAs with p<0.05 in t-test were selected as biomarkers.

FIG. 7 depicts comparison of expression levels of miRNAs in SLE patients with or without comorbidity B according to one embodiment of the present disclosure. miRNAs with p<0.05 in t-test were selected as biomarkers.

FIG. 8 depicts comparison of expression levels of miRNAs in SLE patients with or without comorbidity C according to one embodiment of the present disclosure. miRNAs with p<0.05 in t-test were selected as biomarkers.

FIG. 9 depicts comparison of expression levels of miRNAs in SLE patients with or without comorbidity D according to one embodiment of the present disclosure. miRNAs with p<0.05 in t-test were selected as biomarkers.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

### Definitions

Unless otherwise indicated, all terms used herein have the same meaning as they would to one skilled in the art.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

“About,” as used herein, refers broadly to up to a 5% variance in a given numeric value.

“Array,” as used herein, refers broadly to a population of targets, such as miRNAs, that can be attached to a surface in a spatially distinguishable manner. An individual feature of an array can comprise a single copy of a target, such as a miRNA, or a population of targets, such as miRNA s, at an individual feature of the array. The population of miRNAs at each feature typically is homogenous, having a single species of the particular target. However, in some embodiments a heterogeneous population of miRNAs can be present at a feature. Thus, a feature need not include only a single miRNAs and can instead contain a plurality of different miRNAs.

“Body fluid,” as used herein, refers broadly to any of various types of fluid found in the body of an animal. The bodily fluid may be in a liquid state or in a solid state, e.g., frozen state. The solution may contain a substance to be collected, such as a biomolecule, or may not contain a substance to be collected, and contains a substance for measuring the substance to be collected. The bodily fluid may be a bodily fluid of an animal. The animal may be a reptile, mammal, amphibian. The mammal may be a primate such as a dog, cat, cow, horse, sheep, pig, hamster, mouse, squirrel, and monkey, gorilla, chimpanzee, human. The body fluid may be lymph fluid, tissue fluid, such as interstitial fluid, intercellular fluid, interstitial fluid, and the like, and may be body cavity fluid, serosal fluid, pleural fluid, ascites fluid, capsular fluid, cerebrospinal fluid (cerebrospinal fluid), joint fluid (synovial fluid), and aqueous humor of the eye (aqueous). The body fluid may be digestive fluid, such as saliva, gastric juice, bile, pancreatic juice, intestinal fluid, etc., and may be sweat, tears, runny nose, urine, semen, vaginal fluid, amniotic fluid, milk, etc. The bodily fluids may be collected, extracted, collected, etc. (hereinafter referred to simply as collection) invasively, or may be collected non-invasively.

“Classifier,” as used herein, refers broadly to a machine learning algorithm such as support vector machine(s), AdaBoost classifier(s), penalized logistic regression, elastic nets, regression tree system(s), gradient tree boosting system(s), logistic regression, naive Bayes classifier(s), neural nets, Bayesian neural nets, k-nearest neighbor classifier(s), Deep Learning systems, and random forests. This invention contemplates methods using any of the listed classifiers, as well as use of more than one of the classifiers in combination.

“Classification and Regression Trees (CART),” as used herein, refers broadly to a method to create decision trees based on recursively partitioning a data space so as to optimize some metric, usually model performance.

“Classification system,” as used herein, refers broadly to a machine learning system executing at least one classifier.

“Device,” as used herein, refers broadly to a device used to separate and collect solutes from a solution. In some embodiments, a “device” may be a device used to analyze a substance in a solution. In some embodiments, a “device” may be used to separate organic molecules from solution. In some embodiments, a “device” may be used to separate a biomolecule from a solution. A “device” may be a fluidic device, a flow path device, a combination thereof, or a device including any thereof.

“Elastic Net,” as used herein, refers broadly to a method for performing linear regression with a constraint comprised of a linear combination of the L1 norm and L2 norm of the vector of regression coefficients.

“Extracellular vesicles (EV),” as used herein, refers broadly to vesicles that are released from cells, including those released from cells during apoptosis, and those released from healthy cells. van Niel G et al. “Shedding light on the cell biology of extracellular vesicles.” Nat Rev Mol Cell Biol. (2018) 19(4): 213-228. Extracellular vesicles may be broadly divided into exosomes (exosome), microvesicles (micro vesicle; MV), and apoptotic bodies (apoptosis body), depending on size and surface markers. Exosomes usually have diameters of 40-120 nanometers and may be capable of expressing one or more or all molecules selected from the group consisting of Alix, Tsg101, CD9, CD63, CD81 and flotillin. Exosomes can include proteins and nucleic acids, such as mRNA, miRNA, ncRNA. Microvesicles usually have diameters of 50-1,000 nanometers and may be capable of expressing one or more or all molecules selected from the group consisting of integrins, selectins, and CD40. Microvesicles can include proteins and nucleic acids, such as mRNA, miRNA, ncRNA. Apoptotic bodies usually have a diameter of 500-2,000 nm and may be capable of expressing one or more molecules selected from the group consisting of annexin V and phosphatidylserine. Apoptotic bodies may contain fragmented nuclei and organelles.

“Effective amount,” as used herein, refers broadly to an amount of a composition described herein that is sufficient to produce a desired effect, which can be a therapeutic effect. The exact amount of the composition required for an effective amount will vary from subject to subject, depending on the species, age, weight and general condition of the subject, the severity of the condition being treated, the particular composition used, its mode of administration, the duration of the treatment, the nature of any concurrent treatment, the pharmaceutically acceptable carrier used, and like factors within the knowledge and expertise of those skilled in the art. Thus, it is not possible to specify an exact amount for every composition of this invention. However, an effective amount can be determined by one of ordinary skill in the art in any individual case using only routine experimentation given the teachings herein and by reference to the pertinent texts and literature and/or by using routine experimentation. (See, for example, Remington: The Science and Practice of Pharmacy, 21.sup.st Edition (2005), Lippincott Williams & Wilkins, Philadelphia, PA.).

“False Positive (FP)” and “False Positive Identification,” as used herein, refers broadly to an error in which the algorithm test result indicates the presence of a disease when the disease is actually absent.

“False Negative (FN),” as used herein, refers broadly to an error in which the algorithm test result indicates the absence of a disease when the disease is actually present.

“Free,” as used herein, refers broadly to a biomolecule present in a bodily fluid that is not encapsulated in an extracellular vesicle and is present in an unassociated state with the extracellular vesicle. For example, miRNA in urine or urine extract that is not encapsulated in an extracellular vesicle and is present in an unassociated state with the extracellular vesicle.

“Homologous,” as used herein, refers broadly to the degree of identity (see percent identity above) between sequences of two amino acid sequences, i.e. peptide or polypeptide sequences. The aforementioned “homology” is determined by comparing two sequences aligned under optimal conditions over the sequences to be compared. Such a sequence homology can be calculated by creating an alignment using, for example, the ClustalW algorithm. Commonly available sequence analysis software, more specifically, Vector NTI, GENETYX or other tools are provided by public databases.

“Sequence homology” and “sequence identity,” as are used, may be used interchangeably, and refer broadly to the percentage of sequence homology or sequence identity of amino acid sequences or nucleotide sequences. The sequences may be aligned using computer methods known in the art for optimal comparison purposes. In order to optimize the alignment between the two sequences, gaps may be introduced in any of the two sequences that are compared. Such alignment can be carried out over the full-length of the sequences being compared. Alternatively, the alignment may be carried out over a shorter length, for example over about 5, about 10, about 20, about 50, about 100 or more nucleotides or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison. In D. Sankoff and J. B. Kruskal, (ed.), Time warps, string edits and macromolecules: the theory and practice of sequence comparison, Addison Wesley). The percent sequence identity between two amino acid sequences or between two nucleotide sequences may be determined using the Needleman and Wunsch algorithm for the alignment of two sequences. (Needleman, S. B. and Wunsch, C. D. (1970) J. Mal. Biol. 48, 443-453). Both amino acid sequences and nucleotide sequences can be aligned by the algorithm. The Needleman-Wunsch algorithm has been implemented in the computer program NEEDLE. For the purpose of this invention, the NEEDLE program from the EMBOSS package was used (version 2.8.0 or higher, EMBOSS: The European Molecular Biology Open Software Suite (2000) Rice, Longden, and Bleasby, Trends in Genetics 16, (6) 276-277, emboss.bioinformatics.nl/). For amino acid sequences, EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”. The nucleotide and amino acid sequences of the present invention can further be used as a “query sequence” to perform a search against sequence databases to, for example, identify other family members or related sequences. Such searches can be performed using the NBLAST and XBLAST programs (version 2.0) of Altschul, et al. (1990) J. Mal. Biol. 215:403-10. BLAST nucleotide searches can be performed with the NBLAST program, score=100, word length=12 to obtain nucleotide sequences homologous to polynucleotides of the invention. BLAST protein searches can be performed with the XBLAST program, score=50, word length=3 to obtain amino acid sequences homologous to polypeptides of the invention. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., XBLAST and NBLAST) can be used.

“Inclusion,” as used herein, refers broadly to a form of a biomolecule incorporated in an extracellular vesicle. For example, microRNA incorporated in an extracellular vesicle (either fully or partially inclusive).

“in situ extraction,” as used herein, refers broadly to disrupting EV captured on nanowires using a nanowire-incorporated microfluidic device to extract small molecule RNAs (e.g., microRNAs) in situ, or extracting small molecule RNAs (e.g., microRNAs) captured on nanowires into solutions from nanowires.

“LASSO,” as used herein, refers broadly to a method for performing linear regression with a constraint on the L1 norm of the vector of regression coefficients.

“L1 Norm,” as used herein, is the sum of the absolute values of the elements of a vector.

“L2 Norm,” as used herein, is the square root of the sum of the squares of the elements of a vector.

“Negative Predictive Value (NPV),” as used herein, is the number of true negatives (TN) divided by the number of true negatives (TN) plus the number of false negatives (FP), TP/(TN+FN).

“Neural Net,” as used herein, refers broadly to a classification method that chains together perceptron-like objects to create a classifier.

“Performance score,” as used herein, refers broadly to the distances between predicted values and actual values in the training data. This is expressed as a number between 0-100%, with higher values indicating the predicted value is closer to the real value. Typically, a higher score means the model performs better.

“Positive Predictive Value (PPV),” is the number of true positives (TP) divided by the number of true positives (TP) plus the number of false positives (FP), TP/(TP+FP).

“Random Forest,” as used herein, refers broadly to a bagging method that fits CARTs based on samples from the dataset that the model is trained on.

“Label,” as used herein, refers broadly to any atom or molecule that can be used to provide a detectable (preferably quantifiable) signal. Labels can be attached to a molecule of interest such as a secondary reagent. Labels may provide signals detectable by such non-limited techniques as fluorescence, radioactivity, colorimetry, gravimetry, X-ray diffraction or absorption, magnetism, enzymatic activity, and combinations thereof.

“Nanowire,” as used herein, refers broadly to a rod-like, wire-like structure having a size, such as a cross-sectional shape or diameter on the order of nanometers (e.g., a diameter of 1 to several hundred nanometers).

“Autoimmune disease,” as used herein, refers to a disease that develops as one's own immune system reacts with one's own healthy cells and tissues. Examples of the autoimmune disease may include diseases, such as SLE, multiple sclerosis, rheumatic arthritis, psoriasis, Crohn's disease, leukoderma vulgaris, Behcet's disease, collagenosis, Type I diabetes mellitus, uveitis, Sjoegren syndrome, autoimmune myocarditis, autoimmune liver diseases (e.g., autoimmune hepatitis), autoimmune gastritis, autoimmune thyroid disease, pemphigus, Guillain-Barre syndrome, chronic inflammatory demyelinating polyneuropathy, and HTLV-1-associated myelopathy.

“Mild SLE,” as used herein, refers broadly to mild to moderate flares generally present as rashes, oral ulcers, and arthritis. These flares may be often confined to skin and joints and at times may be also associated with fever and fatigue. Treatment options for mild flares (e.g., malar rash, fatigue, and arthralgia) may include antimalarials (such as hydroxychloroquine 200-400 mg), non-steroidal anti-inflammatories (NSAIDs) and low dose steroids.

“Moderate SLE,” as used herein, refers broadly to moderate flares (e.g., more severe skin, rash, alopecia), moderate doses of steroids may be used Immunosuppressants, such as methotrexate or azathioprine, might be added for a “steroid sparing” effect for those patients who required prednisone>10 mg/day to control symptoms. Antimalarial adjustment options for moderate flares might include maximizing hydroxychloroquine, addition or substitution with quinacrine or a switch to chloroquine. While these medications can help reduce symptoms, improve disease manifestations, and sometimes induce remission, they can also have significant negative side effects. Steroids, in particular, commonly cause insomnia, osteoporosis, muscle weakness, and much more. Belimumab (Benlysta), a monoclonal antibody directed against a soluble B lymphocyte survival factor, e.g., belimumab, has recently been approved for patients in this category.

“microRNA” (also referred to as “miRNA”), as used herein, refers broadly to a type of non-coding RNA (ncRNA) that is believed not to encode proteins. MicroRNAs are processed from their precursors into mature bodies. The mature microRNAs are known to have lengths on the order of 20 to 25 bases. Human microRNAs are named hsa. Precursors are given mir and matures are given miR. The identified sequences are numbered in the order, in which they are identified, and for similar sequences, the numbers are followed by a lower case alphabet. If there is a precursor derived from the 5′ end and a precursor derived from the 3′ end, the microRNAs derived from the 5′ end are labeled with 5p and those derived from the 3′ end are labeled with 3p. These symbols and numbers are connected by hyphens. The mature microRNA may be double-stranded. miRNAs may be important regulators for cell growth, differentiation, and apoptosis, and thus, may be important for normal development and physiology.

“Ridge Regression,” as used herein, refers broadly to a method for performing linear regression with a constraint on the L2 norm of the vector of regression coefficients.

“Severe SLE,” as used herein, refers broadly refers to severe flares refer to life or organ-threatening disease, such as significant kidney disease, brain disease, very low platelet or red blood cell count, vasculitis. For such severe manifestations of SLE, treatment generally starts with pulse solumedrol (1 gram/day IV for 3 days), followed by high dose prednisone 1-2 mg/kg per day. More potent immunosuppressants, such as IV cyclophosphamide (Cytoxan), mycophenolate mofetil (CellCept), azathioprine (Imuran) or recently developed biologic therapies like Benlysta and rituximab (RTX) (trade name Rituxan) may be added.

“SLE Comorbidity,” as used herein, refers broadly to comorbidities associated with SLE. SLE may be associated with a greater risk for cancer, cardiovascular, renal, liver, rheumatological and neurological diseases as well as hypothyroidism, psychosis, and anaemia. The development of comorbidities may be most frequent in the first two years of SLE diagnosis. Vascular disease may be one of the most common of the many comorbidities associated with SLE. In addition to cardiovascular disease, patients with SLE may have a number of other comorbidities, including osteoporosis, Sjoegren's syndrome, antiphospholipid syndrome, autoimmune thyroid disease, malignancies, rheumatoid arthritis, systemic sclerosis, myositis, vasculitis, autoimmune hepatitis, and infections.

“Subject,” as used herein, refers broadly to any animal susceptible to SLE. Such a subject is generally a mammalian subject, including but not limited to human, primate, dog, cat, pig, rabbit, guinea pig, goat, cow, cattle, horse, and the like. Thus, in some embodiments, a subject can be any domestic, commercially or clinically valuable animal including an animal model of SLE. Subjects may be male or female and may be any age including neonate, infant, juvenile, adolescent, adult, and geriatrics objects. In particular embodiments, the subject is a human. The term “subject” and “patient” are used interchangeably.

“Standard of Deviation (SD),” as used herein, is the spread in individual data points (i.e., in a replicate group) to reflect the uncertainty of a single measurement.

“Subset,” as used herein, refer broadly to a proper subset and “superset” is a proper superset.

“Subject in need thereof,” as used herein, refers broadly to a subject known to have, or suspected of having, or at increased risk of developing, SLE. A subject of this invention can also include a subject not previously known or suspected to have SLE or in need of treatment for SLE. A subject of this disclosure is also a subject known to have or believed to be at risk of developing SLE. Subjects described herein as being at risk of developing SLE are identified by family history, genetic analysis, environmental exposure and/or the onset of early symptoms associated with the disease or disorder described herein.

“Separation” and “concentration,” as used herein, refer broadly to methods for the separation of EV from cell culture medium or body fluids with high purity and quality. Separation may refer to purification or isolation of EVs from other non-EV components of the materials (conditioned medium, biofluid, tissue) and the different types of EVs from each other. Concentration may be a means to increase numbers of EVs per unit volume, with or without separation. EV separation and concentration can be achieved by multiple technologies based on EV size or surface marker expression. These techniques may include differential ultracentrifugation, density gradient centrifugation, immunoaffnity, ultrafiltration, polymer-based precipitation, and size-exclusion chromatography.

“Substantially free,” as used herein, refers broadly to the presence of a specific component in an amount less than 1%, preferably less than 0.1% or 0.01%. More preferably, the term “substantially free” refers broadly to the presence of a specific component in an amount less than 0.001%. The amount may be expressed as w/w or w/v depending on the composition.

“Solid support,” “support,” and “substrate,” as used herein, refers broadly to any material that provides a solid or semi-solid structure with which another material can be attached including but not limited to smooth supports (e.g., metal, glass, plastic, silicon, and ceramic surfaces) as well as textured and porous materials. Substrate materials comprise, but are not limited to acrylics, carbon (e.g., graphite, carbon-fiber, nanotubes), ceramics, controlled-pore glass, cross-linked polysaccharides (e.g., agarose or SEPHAROSE(registered trademark)), gels, glass (e.g., modified or functionalized glass), graphite, inorganic glasses, inorganic polymers, metal oxides (e.g., SiO₂, TiO₂, stainless steel), nanomaterials (e.g., highly oriented pyrolitic graphite (HOPG) nanosheets), organic polymers, plastics, polacryloylmorpholide, poly(4-methylbutene), poly(ethylene terephthalate), poly(vinyl butyrate), polybutylene, polydimethylsiloxane (PDMS), polyethylene, polyformaldehyde, polymethacrylate, polypropylene, polystyrene, polyurethanes, polyvinylidene difluoride (PVDF), resins, silica, silicon (e.g., surface-oxidized silicon), or a combination thereof.

“Surface,” as used herein, refers broadly to a part of a support structure (e.g., substrate) that is accessible to contact with reagents, beads or analytes. The surface can be substantially flat or planar. Alternatively, the surface can be rounded or contoured. Exemplary contours that can be included on a surface are wells, depressions, pillars, ridges, channels. The terms “surface” and “substrate” are used interchangeably herein.

“Training Set,” as used herein, is the set of samples that are used to train and develop a machine learning system, such as an algorithm used in the method and systems described herein.

“Treatment,” as used herein, refers broadly to alleviating signs and/or symptoms of a disease or injury condition. Treatment may encompass prophylactic measures, where the therapeutic composition is administered prior to the development of signs and/or symptoms or exposure to the disease or injury condition to lessen the development of signs and/or symptoms of a disease or injury condition.

“True Negative (TN),” as used herein, is the algorithm test result indicates that a miRNA is not associated with SLE when the miRNA is actually associated with SLE.

“True Positive (TP),” as used herein, is the algorithm test result indicates that a miRNA is associated with SLE when the SLE is actually associated with SLE.

“Truncated,” as used herein, refers broadly to a sequence, when polynucleotide, with the 5′ and/or 3′ ends shortened, and, when a polypeptide, where the N- and/or C-end are shortened.

“Urine extract,” as used herein, refers broadly to a product extracted from urine in which certain components, particularly microRNAs, are more concentrated than in the urine prior to extraction.

“Validation Set,” as used herein, refers broadly to the set of samples that are blinded and used to confirm the functionality of the algorithm used in the method and systems described herein. This is also known as the Blind Set.

Systemic Lupus Erythematosus (SLE)

Systemic Lupus Erythematosus (SLE) is a prototypic chronic autoimmune disease affecting multiple organs with an unknown cause. Despite significant research into SLE, effective targeted therapies in SLE are lacking. The existing treatment options to relieve symptoms and control the progression of the disease include drugs that provide nonspecific immunosuppression for keeping the disease under control, e.g., nonsteroidal anti-inflammatory drugs (NSAIDs) and immunosuppressants, such as hydroxychloroquine, corticosteroids, methotrexate, azathioprine, cyclophosphamide, and mycophenolate mofetil. Belimumab is the first ever targeted biological for the treatment of SLE patients with active, autoantibody-positive disease, who are already on standard therapy. Belimumab is a fully human IgG1λ recombinant monoclonal antibody directed against B lymphocyte stimulator (BLyS). Specific binding of belimumab with the soluble BLyS prevents the interaction of BLys with its three receptors and indirectly decreases the B-cell survival and production of autoantibodies.

The symptoms of SLE include, but are not limited to, achy joints/arthralgia, fever of more than 100° F./38° C., arthritis/swollen joints, prolonged or extreme fatigue, skin rashes, anemia, kidney involvement, pain in the chest on deep breathing/pleurisy, butterfly-shaped rash across the cheeks and nose, sun or light sensitivity/photosensitivity, hair loss, blood clotting problems, Raynaud's phenomenon/fingers turning white and/or blue in the cold, seizures, mouth or nose ulcers, and any combination thereof.

The SLE condition may be mild SLE, where the patient suffers from mild to moderate flares generally present as rashes, oral ulcers, and arthritis. These flares may be often confined to skin and joints and at times may be also associated with fever and fatigue. Treatment options for mild flares (e.g., malar rash, fatigue, and arthralgia) may include antimalarials (such as hydroxychloroquine 200-400 mg), non-steroidal anti-inflammatories (NSAIDs) and low dose steroids.

The SLE condition may be moderate SLE, where the patient suffers from moderate flares (e.g., more severe skin, rash, alopecia), moderate doses of steroids may be used. Immunosuppressants, such as methotrexate or azathioprine, might be added for a “steroid sparing” effect for those patients who required prednisone>10 mg/day to control symptoms. Antimalarial adjustment options for moderate flares might include maximizing hydroxychloroquine, addition or substitution with quinacrine or a switch to chloroquine. While these medications can help reduce symptoms, improve disease manifestations, and sometimes induce remission, they can also have significant negative side effects. Steroids, in particular, commonly cause insomnia, osteoporosis, muscle weakness, and much more. Belimumab (Benlysta), a monoclonal antibody directed against a soluble B lymphocyte survival factor, e.g., belimumab, has recently been approved for patients in this category.

The SLE condition may be severe SLE, where the patient suffers from severe flares refer to life or organ-threatening disease, such as significant kidney disease, brain disease, very low platelet or red blood cell count, vasculitis. For such severe manifestations of SLE, treatment generally starts with pulse solumedrol (1 gram/day IV for 3 days), followed by high dose prednisone 1-2 mg/kg per day. More potent immunosuppressants, such as IV cyclophosphamide (Cytoxan), mycophenolate mofetil (CellCept), azathioprine (Imuran) or recently developed biologic therapies like Benlysta and rituximab (RTX) (trade name Rituxan) may be added.

SLE may be associated with other conditions, referred to as “SLE Comorbidity”. SLE may be associated with cancer, a greater risk for cancer, cardiovascular, renal, liver, rheumatological and neurological diseases as well as hypothyroidism, psychosis, and anaemia. The development of comorbidities may be most frequent in the first two years of SLE diagnosis. Vascular disease may be one of the most common of the many comorbidities associated with SLE. In addition to cardiovascular disease, patients with SLE may have a number of other comorbidities, including osteoporosis, Sjoegren's syndrome, antiphospholipid syndrome, autoimmune thyroid disease, malignancies, rheumatoid arthritis, systemic sclerosis, myositis, vasculitis, autoimmune hepatitis, and infections.

As described herein, approximately 22,000 protein-coding transcripts mRNAs (and subsets thereof) may be used to distinguish SLE patients from healthy controls. MicroRNAs represent a purely regulatory, as opposed to structural, process that fine-tunes mRNA expression. The combinatorial nature of nucleotide complementarity permits individual miRNAs to regulate the expression of hundreds of genes by post-transcriptional modification of their cognate messenger RNAs.

The mature microRNA may be double-stranded. miRNAs may be important regulators for cell growth, differentiation, and apoptosis, and thus, may be important for normal development and physiology. Consequently, dysregulation of miRNA function may lead to human diseases, such as cancers, immune diseases, and viral infection. Differential expression of miRNAs may be useful in diagnosing/treating SLE.

miRNA expression may be a richer source of information for pathogenesis of diseases than messenger RNA profiling and thus holds the promise of translating into practice as a mechanism-based molecular biomarker for preventive, predictive, personalized and participatory medicine (“P4 medicine”). See, e.g., Flores et al. “P4 medicine: how systems medicine will transform the healthcare sector and society.” Per Med. (2013) 10(6): 565-576.

Embodiments of the present disclosure comprise identification of SLE patients using biomarkers and treatment of SLE patients based on such identification. For example, the methods described herein may utilize a classifier to identify miRNAs, e.g., identify miRNAs and/or their expression levels associated with SLE from a data set of miRNAs and expression levels. In one embodiment, miRNA data, acquired from the method of detecting miRNA expression levels described herein or described in the art, are assembled into a database and processed by a classifier to a classification of miRNAs and their expression levels as indicative or not indicative of SLE. See, e.g., U.S. Patent Application Publication No. 2020/0255906.

Method of Detecting miRNAs

The methods described herein may comprise obtaining a sample and analyzing the miRNA content in the sample.

The sample may be a body fluid. The body fluid may be blood, urine, plasma, saliva, ascites, bronchoalveolar lavage fluid, cerebrospinal fluid, or a combination thereof. The sample, including body fluids, may be collected by any means known in the art. Extractors, such as syringes, may be used to extract, collect, and collect solution from the subject.

The sample, including a body fluid, may be taken from a subject, including a subject with a particular disease, or may be a bodily fluid of a subject suspected of suffering from a particular disease or a subject to be tested for suffering from a particular disease. In some embodiments, the disease may be immune diseases, such as SLE.

The sample may be an urine extract may be an aqueous solution (solution or suspension), or it may be a solid obtained by drying the urine sample. In urine extracts, extracts from which components other than the extracellular vesicles and nucleic acids in the urine have been substantially removed may also be referred to as urine purifications. The urine extract may comprise a surfactant, preferably a non-ionic surfactant. The urine extract may comprise detergents and debris of extracellular vesicles (e.g., exosomes and/or microvesicles). The urine extract may be free or substantially free of one or more selected from the group consisting of detergents and debris of extracellular vesicles (e.g., exosomes and/or microvesicles). The urine extract may further comprise a stabilizing agent (e.g., a nucleic acid stabilizing agent) and/or a pH adjusting agent (e.g., a buffering agent). The urine extract may comprise salts. The urine extract may comprise a urine component, e.g., one or more urine components selected from the group consisting of urea, creatinine, uric acid, ammonia, urobilin, riboflavin, urinary protein, sugar and urinary hormones (e.g., chorionic gonadotropin). The pH of the urine extract may be equal to or greater than, or greater than, a value such as 2, 3, 4, or 5. The pH of the urine extract may be equal to or less than, or less than, a value such as 10, 9, 8, 7, 6, or 5. The urine extract comprises microRNAs. In the present disclosure, the urine extract may comprise enriched/concentrated microRNAs or groups thereof. In the present disclosure, the urine extract may comprise microRNAs extracted by the extraction methods described herein.

The methods described herein may comprise
(a) obtaining a body fluid sample from a patient suspected of having SLE,
(b) analyzing miRNA expression in the obtained sample, and
(c) identifying the patient
(i) as having the marker correlated with a comorbidity of SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample, or
(ii) as not having the marker correlated with a comorbidity of SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

The methods described herein may comprise analyzing comprising generating an miRNA profile from the sample comprising:
(a) introducing the obtained body fluid sample into a fluidic device comprising a nanowire,
(b) capturing extracellular vesicles in the body fluid sample on the nanowire,
(c) disrupting the captured extracellular vesicles,
(d) extracting at least one miRNA from disrupted extracellular vesicles,
(e) hybridizing the extracted miRNA to an miRNA array; and,
(f) determining miRNA hybridization to the array.

Extracellular vesicles may be broadly divided into exosomes (exosome), microvesicles (micro vesicle; MV), and apoptotic bodies (apoptosis body), depending on size and surface markers. Exosomes usually have diameters of 40-120 nanometers and may be capable of expressing one or more or all molecules selected from the group consisting of Alix, Tsg101, CD9, CD63, CD81 and flotillin. Exosomes can include proteins and nucleic acids, such as mRNA, miRNA, ncRNA. Microvesicles usually have diameters of 50-1,000 nanometers and may be capable of expressing one or more or all molecules selected from the group consisting of integrins, selectins, and CD40. Microvesicles can include proteins and nucleic acids, such as mRNA, miRNA, ncRNA. Apoptotic bodies usually have a diameter of 500-2,000 nm and may be capable of expressing one or more molecules selected from the group consisting of annexin V and phosphatidylserine. Apoptotic bodies may contain fragmented nuclei and organelles.

Extracellular vesicle (EV) separation and concentration can be achieved by multiple technologies based on EV size or surface marker expression. These techniques may include differential ultracentrifugation, density gradient centrifugation, immunoaffnity, ultrafiltration, polymer-based precipitation, and size-exclusion chromatography. Differential centrifugation may be a common approach for EV separation. Briefly, samples may be first centrifuged at low speed to remove cells (500×g). Then, cell debris may be removed after centrifugation at 2500×g. The supernatant may be collected and then centrifugation may be performed at 10,000×g to pellet large EVs, such as microvesicles. The final supernatant may be then ultracentrifuged at 100,000×g to pellet the small EVs that may correspond to exosomes. The final pellet may be then washed in a large volume of phosphate buffered solution (PBS) to eliminate contaminating proteins, then centrifuged one last time at 100,000×g. To achieve better specificity of EV or EV subtype separation, one or more additional techniques may be used. Density gradient centrifugation (velocity or flotation) could further improve EV purity. Exosomes may be purified in a buoyant density using a discontinuous gradient of a sucrose solution or iodixanol cushion. Additional purification can be achieved by immunoaffnity as well. Antibodies (CD63, CD81, CD9) may be conjugated with magnetic beads and incubated with EV-containing samples. EVs can be separated by ultrafiltration based on their size. Common filter pore sizes may be 0.8 μm and 0.22 μm. EVs can be separated by polymer-based precipitation. For example, hydrophilic polymers may be reacted with a solution containing EVs to reduce a solubility of EVs, and the precipitated EVs by centrifugation can be separated. Separation by the polymer-based precipitation can be done, using methods well known to those skilled in the art (for example, Coumains et al. (2017) “Methodological Guidelines to Study Extracellular Vesicles”) and commercially available kits (for example, Total Exosome Isolation Reagent (ThermoFisher)). Some commercial products can also use polyether and its derivates, such as polyethylene glycol (PEG) for precipitation to isolate EVs. Size-exclusion chromatography can separate EV particles by their sizes. To confirm the purity of separated EVs electron microscopy, nanoparticle tracing analysis (NTA), and western blotting may be performed to characterize EV shape, size, and biomarker expression. At least three positive protein markers (such as CD63, CD9, CD81, TSG101, etc.) and a negative protein marker may be necessary (such as calnexin) to define EVs. A single EV could be characterized through two different but complementary techniques: microscopy (such as scanning-probe microscopy, atomic force microscopy, or super-resolution microscopy) or single particle analyzers (NTA, high resolution flow cytometry, and dynamic light scattering).

Microfluidic Chips for EV Separation and Analysis

To enhance the capture efficiency for EVs on microfluidic devices, nanostructures, for example, nanowires, may be designed on chips to provide a larger surface area that may allow direct incorporation of capture antibodies. The nanowire may be a structure whose maximum, minimum, average, or other distinctive sizes in a section may be at the nanometer, sub-nanometer, 10 nanometer, 100 nanometer, or sub-micrometer levels, unless the diameter or distinctive size is defined.

The length of the nanowire may be a longitudinally defined size and may be from a nanometer level to a 10 nanometer level, a 100 nanometer level, or a sub-micrometer level. In one aspect, the length of the nanowires described herein may be from about 0.1 nanometers to about 500 nanometers, from about 1 nanometer to about 250 nanometers, from about 1 nanometer to about 100 nanometers, or from about 5 nanometers to about 50 nanometers. The length of the nanowire may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 200, 201, 202, 203, 204, 205, 206, 207, 208, 209, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nanometers (nm). The length of the nanowire may be between about 1 and 500 nm, 100 and 500 nm, 200 and 400 nm, 250 and 500 nm, 50 and 250 nm, 10 and 100 nm, 2 and 200 nm, 300 and 500 nm, 400 and 500 nm, 150 and 450 nm, 250 and 300 nm, 10 and 50 nm, 100 and 350 nm, 350 and 500 nm, or 200 and 300 nm.

The length of the nanowires may be greater than, for example, but not limited to, values of 500 nm, 1 μm, 1.5 μm, 2 μm, 3 μm, 4 μm, 5 μm, 6 μm, 7 μm, 8 μm, 9 μm, 10 μm, 11 μm, 12 μm, 13 μm, 14 μm, 15 μm, 17 μm, 20 μm, etc. The length of the nanowires may be, for example, but not limited to, equal to or less than 1 μm, 1.5 μm, 2 μm, 3 μm, 4 μm, 5 μm, 6 μm, 7 μm, 8 μm, 9 μm, 10 μm, 11 μm, 12 μm, 13 μm, 14 μm, 15 μm, 17 μm, 20 μm, 50 μm, 100 μm, or 200 μm.

The length of the nanowire may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 μm. The length of the nanowire may be between about 1 and 100 μm, 100 and 200 μm, 120 and 140 μm, 150 and 175 μm, 5 and 25 μm, 10 and 10 μm, 2 and 20 μm, 30 and 100 μm, 15 and 125 μm, 10 and 45 μm, 25 and 180 μm, 60 and 75 μm, 1 and 150 μm, 35 and 200 μm, or 2 and 180 μm.

The diameter (or size in the thickness direction) of the nanowires may be equal to or larger than, e.g., 5 nm, 10 nm, 15 nm, 20 nm, 25 nm, 30 nm, 40 nm, 50 nm, 60 nm, 70 nm, 80 nm, 90 nm, 100 nm, 150 nm, 200 nm, 250 nm, 300 nm, 400 nm, 500 nm, etc. The diameter (or size in the thickness direction) of the nanowires may be equal to or smaller than, e.g., 10 nm, 15 nm, 20 nm, 25 nm, 30 nm, 40 nm, 50 nm, 60 nm, 70 nm, 80 nm, 90 nm, 100 nm, 150 nm, 200 nm, 250 nm, 300 nm, 400 nm, 500 nm, 1 μm.

The diameter of the nanowires may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 200, 201, 202, 203, 204, 205, 206, 207, 208, 209, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nanometers (nm). The length of the nanowire may be between about 1 and 500 nm, 100 and 500 nm, 200 and 400 nm, 250 and 500 nm, 50 and 250 nm, 10 and 100 nm, 2 and 200 nm, 300 and 500 nm, 400 and 500 nm, 150 and 450 nm, 250 and 300 nm, 10 and 50 nm, 100 and 350 nm, 350 and 500 nm, or 200 and 300 nm.

The cross-section of the nanowires may be substantially circular, elliptical, regular polygonal, polygonal, hollow body. The outer shape of the nanowires may be substantially cylindrical, elliptical or polygonal. The nanowires may be hollow or hollow bodies or may be substantially material-packed structures. The nanowire may be formed of one material or a plurality of materials. The nanowire may be coated on its surface with a coating material.

The material of the nanowires may be an inorganic material or an organic material. The nanowires may be or comprise metals, non-metals, semiconductors, mixtures or alloys thereof, or oxides or nitrides thereof. The material of the nanowire may be or comprise a polymeric material. The nanowires may be wires, whiskers, fibers, mixtures or composites thereof. Metals used for the materials of the nanowires may comprise, but are not limited to, typical metals (alkali metals: Li, Na, K, Rb, Cs, alkaline earth metals: Ca, Sr, Ba, Ra), magnesium group elements: Be, Mg, Zn, Cd, Hg, aluminum group elements: Al, Ga, In, rare earth elements: Y, La, Ce, Pr, Nd, Sm, Eu, tin group elements: Ti, Zr, Sn, Hf, Pb, Th, iron group elements: Fe, Co, Ni, earth elements: V Nb, Ta, chromium group elements: Cr, Mo, W, Au, Cu, copper group elements. Rh, Pd, Os, Ir, Pt, natural radioactive elements: U and Th-based radioactive decay products: U, Th, Ra, Rn, actinoids, transuranic elements: Np, Pu, Am, Cm, Bk, Cf, Es, Fm, Md, No, etc., uranium or later, or alloys thereof. The nanowire may be an oxide of any one of the above metals or alloys, or an alloy or mixture, and may comprise an oxide. The material of the nanowires, or at least the surfaces of the nanowires, e.g., cladding, may be, for example, without limitation, ZnO, SiO₂, Li₂O, MgO, Al₂O₃, CaO, TiO₂, Mn₂O₃, Fe₂O₃, CoO, NiO, CuO, Ga₂O₃, SrO, In₂O₃, SnO₂, Sm₂O₃, and EuO. The nanowires may be charged. The nanowires may have a charge opposite to that of the material to be collected or extracted. Thereby, by way of non-limiting example, charged biomolecules, such as extracellular vesicles, nucleic acids, etc. can be efficiently attracted and adsorbed.

The substrate exemplarily may comprise, but is not limited to, semiconductors, metals, insulators, organic materials, polymeric materials, and the like. In one aspect, the substrate can have any shape of structure, e.g., a planar structure, in which the major surfaces may be parallel to each other, a curved structure, in which the major surfaces may not be parallel to each other, or a combination thereof. The substrate may have a three-dimensional structure. The substrate may be formed of a material, on which a catalyst layer can be stacked, e.g. semiconductor materials, such as silicon, quartz glass, glass materials, such as Pyrex (registered trademark) glass, ceramics, polymer material comprise plastic, and the like may be used. In some embodiments, the substrate may be substantially flexible and may be stretchable. In some embodiments, the substrate may be substantially non-flexible. The material of the substrate may be not particularly limited to, and may be, a material selected from polyethylene, polypropylene, polyvinylchloride, polyvinylidene chloride, polystyrene, polyvinyl acetate, polytetrafluoroethylene, ABS (acrylonitrile butadiene styrene) resin, AS (acrylonitrile styrene) resin, thermoplastic resin such as acrylic resin (PMMA), phenolic resin, epoxy resin, melamine resin, urea resin, unsaturated polyester resin, alkyd resin, polyurethane, polyimide, silicone rubber, polymethylmethacrylate (PMMA), and polycarbonate (PC).

Nanowires may be disposed on a substrate (also referred to as a nanowire substrate) and “cover” or “cover member” may be used to mean a different substrate than the substrate on which nanowires are disposed, the member being bonded to the nanowire substrate and being used to form a fluid chamber or flow path.

The nanowire may be attached to a substrate. The nanowire may be situated in a chamber or well.

The substrate for the array used in the systems and methods described herein can be any material that provides a solid or semi-solid structure with which another material can be attached including but not limited to smooth supports (e.g., metal, glass, plastic, silicon, and ceramic surfaces) as well as textured and porous materials. Substrate materials include, but are not limited to acrylics, carbon (e.g., graphite, carbon-fiber, nanotubes), ceramics, controlled-pore glass, cross-linked polysaccharides (e.g., agarose or SEPHAROSE(registered trademark)), gels, glass (e.g., modified or functionalized glass), graphite, inorganic glasses, inorganic polymers, metal oxides (e.g., SiO₂, TiO₂, stainless steel), nanomaterials (e.g., highly oriented pyrolitic graphite (HOPG) nanosheets), organic polymers, plastics, polacryloylmorpholide, poly(4-methylbutene), poly(ethylene terephthalate), poly(vinyl butyrate), polybutylene, polydimethylsiloxane (PDMS), polyethylene, polyformaldehyde, polymethacrylate, polypropylene, polystyrene, polyurethanes, polyvinylidene difluoride (PVDF), resins, silica, silicon (e.g., surface-oxidized silicon).

Substrates need not be flat and can include any type of shape including spherical shapes (e.g., beads) or cylindrical shapes (e.g., fibers). The nanowires attached to solid supports may be attached to any portion of the solid support (e.g., may be attached to an interior portion of a porous solid support material).

Substrates may be patterned where the nanowires attached the substrate are arranged in a pattern. The pattern, e.g., stripes, swirls, lines, triangles, rectangles, circles, arcs, checks, plaids, diagonals, arrows, squares, or cross-hatches, may be etched, printed, treated, sketched, cut, carved, engraved, imprinted, fixed, stamped, coated, embossed, embedded, or layered onto a substrate to allow the nanowires to be arranged in the pattern on the substrate.

The surface of the nanowire may be positively charged. Thus, for example, negatively charged extracellular vesicles can be efficiently collected. For example, the nanowires may be formed of a positively charged material such as ZnO, nickel oxide, or the nanowires may be coated with such a material.

Device

A device can be used to separate extracellular vesicles from the sample, for example blood, plasma, or urine.

The device described herein, which may be used with the methods described herein, may be a microfluidic device comprising:
(a) a sample input in fluid communication with (b)
(b) a separation means, optionally a membrane, filter, at least one nanowire, or combination thereof, in fluid communication with (c) or (d)
(c) a waste chamber or
(d) waste output.

The device described herein, which may be used with the methods described herein, may be a solid substrate comprising a plurality of wells, each well comprising at least one nanowire, optionally, an array comprising nanowires.

The device described herein, which may be used with the methods described herein, may be a solid substrate comprising a plurality of chambers, optionally in fluid communication with each other, each chamber comprising at least one nanowire, optionally, an array comprising nanowires.

The device described herein may comprise a cover over the wells or chambers, optionally a cover that may be removable.

The sample may be introduced into a sample input by, for example, a syringe, syringe pump.

The sample input is fluidly coupled to a separation means including but not limited to a membrane, filter, at least a nanowire, or combination thereof, that allows capture of the extracellular vesicles. After the sample is passed through the separation means, the extracellular vesicles are contacted with a membrane, filter, a nanowire, or combination thereof, capturing the extracellular vesicles on the membrane, filter, a nanowire, or combination thereof. The captured extracellular vesicles may be examined, including by microscopy and/or imaging means.

After the sample has been introduced, the nanowire may be washed with a buffer to remove any unreacted extracellular vesicles and other materials. The extracellular vesicles adsorbed to the nanowires can be analyzed.

When the sample adsorbed on the nanowires of the device is observed with an optical microscope or an electron microscope, the cover may be peeled off from the substrate. When the substrate and the cover member are in close contact with each other with an adhesive, the cover member may be removed, for example by cutting with a blade. Microscopic observation can, for example, determine the size and number of captured samples. Also, quantitative analysis of the surface protein of the captured sample can be performed, for example, by binding an optical label, such as a fluorescent label, to the sample.

For example, a urine extract may be obtained by contacting urine with a nanowire having a positively charged surface (e.g., a nanowire having at least one surface selected from the group consisting of ZnO, SiO₂, Li₂O, MgO, Al₂O₃, CaO, TiO₂, Mn₂O₃, Fe₂O₃, CoO, NiO, CuO, Ga₂O₃, SrO, In₂O₃, SnO₂, Sm₂O₃, EuO, or a combination thereof) in a pH-environment of urine, then (optionally) washing, and extracting the urine extract with a buffer comprising a nonionic surfactant to produce an urine extract. Urine may also be pH adjusted such that the surface charge of the nanowires is positive when contacting the nanowires with urine, before, after, or during contact.

Detection of Extracellular Vesicle (EV)

After introducing EV into the device comprising a nanowire, the nanowire comprising the extracellular vesicle may be washed with a buffer to remove any extracellular vesicles not captured by the nanowire and any other extraneous material(s).

The buffer may be any an isotonic solution, e.g., normal saline solution, buffered saline solution, lactated Ringer's solution, 5% dextrose in water (D5W), Ringer's solution, or 0.9% saline solution. The buffer may be a mineral buffer, balanced saline solution (BSS), TRIS buffer solution (TBS), phosphate buffered saline (PBS), organic buffers, borate buffer solution, carbonate buffer solution, carbonate buffered solution, citrate buffer solution, glycine buffer solution, TRIS buffered saline, Dulbecco's Phosphate saline buffer (DPBS), Dulbecco's Eagle Media (DMEM), Hank's Balanced Salts and Saline Solution (HBSS), Tyrode's Balanced Salts and Saline Solutions (TBSS), Minimum Essential Media, Eagle Basal Medium (EBM), Earle's Balanced Salts and Solutions (EBSS), Puk's Saline, Krebs-Ringer Bicarbonate Buffer, Krebs-Henseleit Buffer, Gey's Balanced Salt Solution (GBSS), Good's Buffers, ACES Buffer, BES Buffer, Bicine Buffer, Bis-Tris Buffer, CAPS Buffer, CAPSO Buffer, CHES Buffer, Glycyl-Glycyl Buffer, MES Buffer, HEPES Buffer, MOPS Buffer, Imidazole Buffer, Succinic Acid Buffer, or a combination thereof.

After washing with a buffer, a buffer (including those described herein) comprising a blocking agent may be introduced and allowed to incubate for about 1-60 minutes. The blocking agent may be bovine serum albumin (BSA), non-fat dry milk (NI-DM), fish gelatin, whole sera, or a polymer including but not limited to polyethylene glycol (PEG), polyvinyl alcohol (PVA), and polyvinylpyrrolidone (PVP). The blocking agent may be used in a concentration of about 0.1% to 10%, for example 1% or 4%.

For example, a blocking solution comprising buffer with 1% bovine serum albumin (BSA) may be introduced and incubated for about 15 minutes. The device may be incubated with the buffer comprising a blocking agent at a temperature between about −20° C. to 25° C. Following incubation with the buffer comprising a blocking agent, the devices may be washed with a buffer and incubated with an antibody that binds the extracellular vesicle. This primary antibody may be visualized using a secondary antibody using methods known in the art.

For example, the devices may be washed with PBS and Alexa Fluor 488 labeled mouse anti-human CD63 monoclonal antibody (10 μm g/ml) or mouse anti-human CD81 monoclonal antibody (10 μm g/ml) may be introduced into the devices, and allow to stand for 15 minutes. For detecting CD81, the devices may be washed and then a Alexa Fluor488 labeled goat-anti-mouse IgG polyclonal antibody may be introduced into the devices as a secondary antibody, and then allow to stand for 15 minutes. Finally, the devices may be washed with PBS and the fluorescence intensity may be observed under a fluorescent microscope. PBS may be used instead of EV samples to obtain background values. For detection using 96-well plates, EV samples may be injected into the holes of the plate and allow to stand for 6 hours, after which the holes may be washed with PBS. 1% BSA solution may be introduced into the holes of the plate and allow to stand for 90 minutes. The wells may then be washed with PBS and Alexa Fluor 488 labeled mouse anti-human CD63 monoclonal antibody (10 μg/ml) or mouse anti-human CD81 antibody (10 μg/ml) may be introduced into the wells of the plates and allow to stand for 45 minutes. For the CD81 detection, in addition to this, the holes of the plate may be washed with PBS, and then a goat-anti-mouse IgG polyclonal antibody (5 μg/ml) labeled with Alexa Fluor 488 may be introduced as a secondary antibody into the holes of the plate, and then allow to stand for 45 minutes. Finally, the holes of the plate may be washed with PBS, and the fluorescent intensities may be observed using a plate reader. PBS may be used instead of EV samples to obtain background values.

miRNA Detection

Detection of microRNAs can be performed using miRNA detection methods known to those skilled in the art, such as quantitative polymerase chain reaction (PCR), microarrays for miRNA detection, RNA-Seq, (e.g., next generation sequencing (NGS)), and multiplex miRNA profiling, and the like. The samples, including urine or urine extract may comprise, for example, 500 or more species of miRNA. Therefore, in order to confirm the expression of all of these miRNA, for example, a microarray for detecting miRNA, a RNA-Seq method, a multiplex miRNA profiling method, can be used. Quantitative PCR-based methods, multiplex miRNA profiling methods can also be used to detect one or more of particular miRNAs in urine or urine extract.

The RNA-seq methods may comprise preparing complementary DNA (cDNA) library and analyzing oligonucleotide sequence of the cDNA library. The cDNA library can be prepared by reverse transcription PCR using total RNA containing miRNA as template. For example, adapters may be allowed to bind specifically to 3′ terminus and 5′ terminus of miRNA, and cDNA may be synthesized through reverse transcription with primers. Here, impurities may be removed from synthesized cDNA using magnetic beads or other means. Then, synthesized cDNA may be amplificated. During cDNA synthesis from miRNA, index sequences unique to each miRNA and universal sequences identified by primes may be comprised in cDNA. In the case, cDNA comprising index sequence may be amplified and a cDNA library may be prepared. Here, impurities may be removed from amplified cDNA using magnetic beads or other means. Preparation of cDNA library can be done, using methods well known to those skilled in the art and commercially available kits (for example, QIAseq miRNA Library Kit (QIAGEN), TaqMan™ Advanced miRNA cDNA Synthesis Kit (ThermoFisher), microScript microRNA cDNA Synthesis kit (Norgen Biotek Corp.) and so on). Prepared cDNA library can be applied for next generation sequencing system (NGS), and miRNA in body fluid sample can be detected.

In the methods described herein, the detection and quantification of miRNA markers of the present disclosure in a subject can be carried out according to methods well known in the art. For example, RNA may be obtained from any suitable sample from the subject that may contain RNA and the RNA may be then prepared and analyzed according to well-established protocols for the presence and/or identification of miRNA(s) according to the methods of this disclosure.

The purified miRNAs may be labeled using methods known in the art. Thus, for example, the labeling can be done using a mirVana™miRNA Labeling Kit (Ambion) and the amine-reactive dyes as recommended by the manufacturer Amine-modified miRNAs can be cleaned up and coupled to NHS-ester modified Cy5 or Cy3 dyes (Amersham Bioscience). The SLE samples may be labeled with Cy5 and healthy controls will be labeled with Cy3. Unincorporated dyes may be removed and the samples hybridized in duplicate according to methods known to those of skill in the art. Thus, for example, the mirVana™ miRNA Bioarrays (Ambion) kit can be used according to the manufacturer's instructions.

Nucleotide sequence that hybridizes to a nucleotide sequence that is complementary to that encoding one of the miRNA sequences disclosed herein (SEQ ID NOs: 1-484) under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art. See, for example, Ausubel, F. M. et al. eds., 1989, Current Protocols in Molecular Biology, Vol. I, Green Publishing Associates, Inc. and John Wiley & Sons, Inc., New York at pages 6.3.1-6.3.6 and 2.10.3.

Detection and analysis of the miRNA by the microarray can comprise labeling the miRNA (e.g., using a fluorescent label as the label), preparing a solution for hybridization, hybridizing the miRNA in the sample with miRNA detection reagents, such as nucleic acids on the microarray, washing the microarray, and then measuring the amount of label (e.g., amount of fluorescence). Quality of the extracted RNA samples can be confirmed by using, for example, methods well known to those skilled in the art or commercially available equipment and kits (e.g., Agilent 2100 Bioanalyzer and RNALabChip from Agilent Technologies, Inc.), with the appearance of peaks between 20 and 30 nucleotides in sizes, as indicator. Labeling of the miRNA can be done, for example, using methods well known to those skilled in the art and commercially available kits (e.g., 3D-Gene™ miRNA labeling kit (Toray Corporation). Also, for example, miRNA analyses by microarrays can be performed using the 3D-Gene™ Human/Mouse/Rat/4animal miRNA Olico chip-4 plex manufactured by Toray Corporation in accordance with the manufacturer's instructions for the products.

Microarray for detecting microRNAs can be a microarray containing probes for one or more selected from the group of microRNAs that exhibit higher expression in one, two, or three patients suspected to have SLE than any of the one, two, or three healthy individuals. A microarray may comprise probes for one or more of the groups of microRNAs (e.g., 1.01 times or more, 1.02 times or more, 1.03 times or more, 1.04 times or more, 1.05 times or more, 1.06 times or more, 1.07 times or more, 1.08 times or more, 1.09 times or more, 1.1 times or more, 1.2 times or more, 1.3 times or more, 1.4 times or more, 1.5 times or more, 1.6 times or more, 1.7 times or more, 1.8 times or more, 1.9 times or more, 2 times or more, 3 times or more, 4 times or more, 5 times or more, 6 times or more, 7 times or more, 8 times or more, 9 times or more, or 10 times or more) that exhibit higher expression in a SLE patient than in a healthy individual. A microarray may comprise probes for one or more of the groups of microRNAs (e.g., 0.99 times or less, 0.98 times or less, 0.97 times or less, 0.96 times or less, 0.95 times or less, 0.94 times or less, 0.93 times or less, 0.92 times or less, 0.91 times or less, 0.9 times or less, 0.8 times or less, 0.7 times or less, 0.6 times or less, 0.5 times or less, 0.4 times or less, 0.3 times or less, 0.2 times or less, 0.1 times or less, 0.09 times or less, 0.08 times or less, 0.07 times or less, 0.06 times or less, 0.05 times or less, 0.04 times or less, 0.03 times or less, 0.02 times or less, or 0.01 times or less) that exhibit lower expression in a SLE patient than in a healthy individual.

In an aspect, the species of the microRNA to be detected (i.e., the kinds of the probes mounted on the microarray) can be, for example, 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, 1000 or more, 1500 or more, 2000 or more, 2500 or more, or 3000 or more.

In another aspect, the species of microRNA to be detected (i.e., the kinds of probes mounted on the microarray) can be, for example, 3000 or less, 2500 or less, 2000 or less, 1900 or less, 1800 or less, 1700 or less, 1600 or less, 1500 or less, 1400 or less, 1300 or less, 1200 or less, 1100 or less, 1000 or less, 900 or less, 800 or less, 700 or less, 600 or less, 500 or less, 400 or less, 300 or less, 200 or less, 100 or less, 90 or less, 80 or less, 70 or less, 60 or less, 50 or less, 40 or less, 30 or less, 20 or less, or 10 or less.

Probes for microRNAs in microarrays can be nucleic acids and derivatives thereof capable of hybridizing to the microRNAs, and can be appropriately designed by those skilled in the art. For example, probes for miRNAs indicative of SLE may comprise ribonucleotide sequences that hybridize to the miRNA of the ribonucleotide sequence of SEQ ID NOs: 1-484 and combinations thereof.

Prior to the detection of the miRNAs contained in an extracellular vesicle, the extracellular vesicles may be disrupted by incubation with a cytolysis buffer, alkali/detergent pre-treatment, storage at about −25° C. for 1-10 days, preferably about 7 days, or a combination thereof. Further the extracellular vesicles may be disrupted using electric field-induced disruption as described in Wang et al. Methods Mol Biol. (2017) 1660: 367-376.

For example, the alkali/detergent pre-treatment may comprise treating the sample at 0.4 N NaOH together with 0.5% Triton X-305 for about 20 minutes, incubation with 0.01% sodium dodecyl sulfate (SDS) for 10 min to disrupt EV membranes.

Classification Systems

Exemplary classification systems used in diagnosing and predicting the occurrence of a medical condition may include those described in U.S. Pat. Nos. 7,321,881; 7,467,119; 7,505,948; 7,617,163; 7,676,442; 7,702,598; 7,707,134; 7,747,547; and 9,952,220, which are each hereby incorporated by reference in their entirety.

The invention relates to, among other things, characterizing miRNA based on data comprising experimental miRNA expression data sets from healthy and patients with SLE, including different severities of SLE. The miRNA expression data sets may be propriety or accessed from publicly available databases.

The classification systems used herein may include computer executable software, firmware, hardware, or combinations thereof. For example, the classification systems may include reference to a processor and supporting data storage. Further, the classification systems may be implemented across multiple devices or other components local or remote to one another. The classification systems may be implemented in a centralized system, or as a distributed system for additional scalability. Moreover, any reference to software may include non-transitory computer readable media that when executed on a computer, causes the computer to perform a series of steps.

The classification systems described herein may include data storage such as network accessible storage, local storage, remote storage, or a combination thereof. Data storage may utilize a redundant array of inexpensive disks (“RAID”), tape, disk, a storage area network (“SAN”), an internet small computer systems interface (“iSCSI”) SAN, a Fibre Channel SAN, a common Internet File System (“CIFS”), network attached storage (“NAS”), a network file system (“NFS”), or other computer accessible storage. The data storage may be a database, such as an Oracle database, a Microsoft SQL Server database, a DB2 database, a MySQL database, a Sybase database, an object oriented database, a hierarchical database, Cloud-based database, public database, or other database. Data storage may utilize flat file structures for storage of data. Exemplary embodiments used two Tesla K80 NVIDIA GPUs, each with 4992 CUDA cores and large amounts of GB of memory (e.g., over 11 GB) to train the deep learning algorithms.

In the first step, a classifier is used to describe a pre-determined set of data. This is the “learning step” and is carried out on “training” data.

The training database is a computer-implemented storage of data reflecting a plurality of miRNA expression data for a plurality of miRNAs with a classification with respect to SLE and/or SLE severity of each respective miRNA. The miRNA expression data may comprise miRNA expression data, predicted miRNA expression data, or a combination thereof. The format of the stored data may be as a flat file, database, table, or any other retrievable data storage format known in the art. The test data may be stored as a plurality of vectors, each vector corresponding to an individual miRNA, each vector including a plurality of miRNA expression data measures for a plurality of miRNA expression data together with a classification with respect to SLE and/or SLE severity characterization of the miRNA. The vector may further comprise miRNA expression data measures for a plurality of experimental miRNA expression data together with a classification with respect to the SLE and/or SLE severity characterisation of the miRNA. Typically, each vector contains an entry for each miRNA expression data measure in the plurality of miRNA expression data measures. The entry may further comprise miRNA presence or absence in different bodily fluid data. The training database may be linked to a network, such as the internet, such that its contents may be retrieved remotely by authorized entities (e.g., human users or computer programs). Alternately, the training database may be located in a network-isolated computer. Further, the training database may be Cloud-based, including proprietary and public databases containing miRNA expression data (e.g., experimental, predicted, and combinations thereof) for miRNAs useful in the diagnosis of SLE.

In the second step, which is optional, the classifier is applied in a “validation” database and various measures of accuracy, including sensitivity and specificity, are observed. In an exemplary embodiment, only a portion of the training database is used for the learning step, and the remaining portion of the training database is used as the validation database. In the third step, miRNA expression data measures from a subject are submitted to the classification system, which outputs a calculated classification (e.g., characterization of a miRNA as associated with SLE and/or SLE severity) for the subject. Additionally, miRNA presence or absence in different bodily fluid data may also be used.

There are many possible classifiers that could be used on the data. Machine and deep learning classifiers include but are not limited to AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, logistic regression model, Random Forests, ridge regression, support vector machines, or an ensemble thereof, may be used to classify the data. See e.g., Han & Kamber (2006) Chapter 6, Data Mining, Concepts and Techniques, 2nd Ed. Elsevier: Amsterdam. As described herein, any classifier or combination of classifiers (e.g., ensemble) may be used in a classification system. As discussed herein, the data may be used to train a classifier. Other classifiers and machine learning systems known in the art may also be used. For example, scikit-learn, a machine learning system in Python computer language may be used.

Scikit-learn (also known as sklearn) is a machine learning library for the Python programming language.

Scikit-Learn uses classification, regression and clustering algorithms including support vector machines, random forests, gradient boosting, k-means and Density-based spatial clustering of applications with noise (DBSCAN) and is designed to interoperate with the Python numerical and scientific libraries NumPy and SciPy.

A preferred classifier is a logistic regression model using the following equation:

(True Positive+True Negative)/(True Positive+True Negative+False Positive+False Negative).

The classifiers described herein may be constructed using a logistic regression-modelled classifier as follows:

$$Pr ( Y ) = \frac{1}{1 + {e}^{- ( α + {β}_{1} {x}_{1} + {β}_{2} {x}_{2} + {β}_{3} {x}_{3} + … + {B}_{2565} {x}_{2565}}} \text{?}$$

$$\text{?} \text{indicates text missing or illegible when filed}$$

Y was a predicted objective variable, x was fluorescence intensities of each miRNA species, b was weight coefficients of each miRNA species, and a was an intercept. In this model, b and a were estimated from each fluorescence intensity of nanowire-extracted urinary miRNA species by supervised machine learning. A value of Y was defined as below 0.5 as non-cancer subjects, and that of Y more than or equal to 0.5 as cancer subjects. The classifier solved the optimization problem for the least-square error term and the L1 regularization term, simultaneously, when fitting logistic regression classifier; 1 acted as an adjuster between the two terms. When 1=1 was used, it showed higher AUC, sensitivity, and specificity values.

Training Data

In another aspect, methods described herein include training of about 75%, about 80%, about 85%, about 90%, or about 95% of the data in the library or database and testing the remaining percentage for a total of 100% data. In an aspect, from about 70% to about 90% of the data is trained and the remainder of about 10% to about 30% of the data is tested, from about 80% to about 95% of the data is trained and the remainder of about 5% to about 20% of the data is tested, or from about 90% of the data is trained and the remainder of about 10% of the data is tested. In an aspect, the database or library contains data from the analysis of over about 20, about 50, over about 100, over about 150, over about 200, or over about 300 miRNAs species. In a further aspect, the library or database includes only verified experimental data, for example from miRNA expression methods. In yet another aspect, the library or database does not include miRNA expression data that were theoretically prepared without the determination of miRNA presence or prevalence by analyzing patient sample. The training data may comprise miRNA expression levels, presence or absence of a miRNA in a bodily fluid, or combinations thereof.

Methods of Classifying Data Using Classification System(s)

The invention provides for methods of classifying data (test data, e.g., miRNA expression levels, presence or absence of a miRNA in a bodily fluid, or combinations thereof) obtained from an individual. These methods involve preparing or obtaining training data, as well as evaluating test data obtained from an individual (as compared to the training data), using one of the classification systems including at least one classifier as described herein. Preferred classification systems use classifiers such as, but not limited to, support vector machines (SVM), AdaBoost, penalized logistic regression, logistic regression, naive Bayes classifiers, classification trees, k-nearest neighbor classifiers, Deep Learning classifiers, neural nets, random forests, Fully Convolutional Networks (FCN), Convolutional Neural Networks (CNN), and/or an ensemble thereof. Scikit-learn is a preferred machine learning library comprising an ensemble of classification, regression, and cluster algorithms including support vector machines, random forests, gradient boosting, k-means and Density-based spatial clustering of applications with noise (DBSCAN). The classification system outputs a classification of the miRNA based on the test data, e.g., miRNA expression levels, presence in a bodily fluid, or a combination thereof.

Particularly preferred for the present invention is an ensemble method used on a classification system, which combines multiple classifiers. For example, an ensemble method may include SVM, AdaBoost, penalized logistic regression, logistic regression, naive Bayes classifiers, classification trees, k-nearest neighbor classifiers, neural nets, Fully Convolutional Networks (FCN), Convolutional Neural Networks (CNN), Random Forests, Deep Learning, or any ensemble thereof, in order to make a prediction regarding miRNA expression correlation with SLE and/or SLE severity. The ensemble method was developed to take advantage of the benefits provided by each of the classifiers, and replicate measurements of each miRNA expression data.

A method of classifying test data, the test data comprising expression data for a miRNA comprising:
(a) accessing an electronically stored set of training data vectors, each training data vector or k-tuple representing an individual miRNA and comprising miRNA expression data for the respective miRNA for each replicate, the training data vector further comprising a classification with respect to miRNA characterization of each respective miRNA;
(b) training an electronic representation of a classifier or an ensemble of classifiers as described herein using the electronically stored set of training data vectors;
(c) receiving test data comprising a plurality of miRNA expression data;
(d) evaluating the test data using the electronic representation of the classifier and/or an ensemble of classifiers as described herein; and
(e) outputting a classification of the miRNA based on the evaluating step.

The test data may further comprise data on the presence or absence of miRNA in a bodily fluid.

In another embodiment, the invention provides a method of classifying test data, the test data comprising miRNA expression data comprising:
(a) accessing an electronically stored set of training data vectors, each training data vector or k-tuple representing an individual human and comprising miRNA expression data for the respective human for each replicate, the training data further comprising a classification with respect to correlation to SLE of each respective miRNA;
(b) using the electronically stored set of training data vectors to build a classifier and/or ensemble of classifiers;
(c) receiving test data comprising a plurality of miRNA expression data for a human test subject;
(d) evaluating the test data using the classifier(s); and
(e) outputting a classification of the human test subject based on the evaluating step.

Alternatively, all (or any combination of) the replicates may be averaged to produce a single value for each miRNA expression data for each subject. Outputting in accordance with this invention includes displaying information regarding the classification of the human test subject in an electronic display in human-readable form. The miRNA data may comprise miRNA expression data, the presence or absence of miRNA in a bodily fluid, or combinations thereof.

The set of training vectors may comprise at least 20, 25, 30, 35, 50, 75, 100, 125, 150, or more vectors.

The test data may be any information measures such as the presence or absence of miRNA in a bodily fluid, miRNA expression data, or a combination thereof.

The data used to train a machine learning system may comprise data from patients with SLE, including at least 5, 10, 15, 20, or 25 different indications, data from normal tissues, including at least about 5, 10, 15, 20, 25, 30, 35, 40, or 45 normal tissues, or a combination thereof. In addition, the data used to train a machine learning system, e.g., Scikit-learn.

It will be understood that the methods of classifying data may be used in any of the methods described herein. In particular, the methods of classifying data described herein may be used in methods for identifying miRNA associated with SLE and/or severity of SLE, for use in diagnostic and therapeutic methods.

Particularly preferred for the present invention is an ensemble method used on a classification system, which combines multiple classifiers. For example, an ensemble method may include Support Vector Machine (SVM), AdaBoost, penalized logistic regression, logistic regression, naive Bayes classifiers, classification trees, k-nearest neighbor classifiers, neural nets, Deep Learning systems, Random Forests, or any combination thereof, in order to make a prediction regarding the association of miRNA with SLE and/or severity of SLE. In addition, the ensemble may be used to make a prediction regarding the association of a miRNA with a type of SLE. The ensemble approach takes advantage of the benefits provided by each of the classifiers, and replicate measurements of each miRNA.

In an aspect, the present disclosure may include a method of classifying test data, the test data containing miRNA expression data, the method including:
(a) receiving, on at least one processor, test data comprising miRNA expression data,
(b) evaluating, using the at least one processor, the test data using a classifier which is an electronic representation of a classification system, each said classifier trained using an electronically stored set of training data vectors, each training data vector representing an individual miRNA and comprising a miRNA expression data for the miRNA, each training data vector further comprising a classification with respect to whether or not the miRNA is indicative of SLE,
(c) outputting, using the at least one processor, a classification of the sample from the miRNA expression data concerning the likelihood of whether or not the miRNA is indicative of SLE based on the evaluating step.

In another aspect, the present disclosure may include a method of classifying test data, the test data comprising miRNA expression data, the method including:
(a) accessing, using at least one processor, an electronically stored set of training data vectors, each training data vector representing an individual patient and comprising a miRNA expression data for the respective patient, each training data vector further comprising a classification with respect to whether or not the miRNA expression is associated with SLE;
(b) training an electronic representation of a classification system, using the electronically stored set of training data vectors;
(c) receiving, at the at least one processor, test data comprising miRNA expression data;
(d) evaluating, using the at least one processor, the test data using the electronic representation of the classification system; and
(e) outputting a classification of the test data concerning whether or not the miRNA expression is associated with SLE based on the evaluating step.

In another aspect, the present disclosure may include a method of classifying test data, the test data containing miRNA expression data, the method including:
(a) accessing, using at least one processor, an electronically stored set of training data vectors, each training data vector representing a severity of SLE and comprising a miRNA expression data for the respective severity of SLE, each training data vector further comprising a classification with respect to whether or not a miRNA is associated with a severity of SLE;
(b) training an electronic representation of a classification system, using the electronically stored set of training data vectors;
(c) receiving, at the at least one processor, test data comprising miRNA expression data;
(d) evaluating, using the at least one processor, the test data using the electronic representation of the classification system; and
(e) outputting a classification of the test data concerning whether or not the miRNA is associated with a severity of SLE based on the evaluating step.

In another aspect, the present disclosure may include a method of classifying test data, including:
(a) obtaining a sample from an individual,
(b) acquiring miRNA expression data in the sample,
(c) comparing the experimental miRNA expression data to miRNA expression data located in a database,
(d) generating a match between the experimental miRNA expression data and the miRNA expression date located in a database,
(e) producing a data set of matched miRNAs based on steps (a), (b), (c), (d), or a combination thereof,
(g) evaluating the data set of miRNAs using a classification system to generate a miRNA expression profile indicative of SLE.

In another aspect, the present disclosure may include a method of classifying test data, including:
(a) obtaining at least one sample from a patient and corresponding sample from a healthy individual,
(b) identifying at least one miRNA in the sample,
(c) generating experimental miRNA expression data from the sample;
(e) comparing the experimental miRNA expression data to miRNA expression date in a database,
(f) generating a match between the experimental miRNA expression data and the miRNA expression date located in a database,
(g) producing a spectral library of miRNA expression data,
(h) evaluating the spectral library of miRNA expression using a classification system to generate a miRNA expression prediction model, and
(i) using the prediction model to generate predicted miRNA expression patterns associated with SLE.

In another aspect, the present disclosure may include a method of classifying test data to identify miRNA associated with SLE, including:
(a) obtaining at least one sample from a patient and corresponding sample from a healthy individual,
(b) identifying at least one miRNA in the sample to produce an experimental miRNA expression data;
(c) comparing experimental miRNA expression data to miRNA expression data in a database; (d) estimation of false discovery rate (FDR);
(e) generation of a match of the experimental miRNA expression data and miRNA expression data in a database;
(f) inputting the data generated by the comparison into a classification system to train a miRNA expression prediction model;
(g) developing predicted miRNA expression pattern; and
(h) identifying an miRNA expression pattern as indicative of SLE.

In another aspect, the database may be a public database, non-public database, or a combination thereof. In another aspect, the miRNA expression data may be experimental miRNA expression data, predicted miRNA expression data, or a combination thereof. In another aspect, the miRNA expression data is experimental miRNA expression data. In another aspect, the test data may further include data on the presence or absence of an miRNA in a bodily fluid. In another aspect, the miRNA expression may be identified using microarray analysis, or a combination thereof.

In another aspect, the classification system may be AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, logistic regression model, Deep Learning, elastic nets, Fully Convolutional Networks (FCN), genetic algorithms, gradient boosting trees, k-nearest neighbor classifiers, LASSO, Linear Classifiers, Naieve Bayes, neural nets, penalized logistic regression, Random Forests, ridge regression, support vector machines, or an ensemble thereof. In another aspect, the classification system may be an ensemble of classification systems.

In another aspect, the library or database may include over about 70%, over about 80%, over about 85%, over about 90%, over about 95%, or 100% miRNA expression data. In another aspect, the miRNA may be identified by the predicted miRNA expression data have an identification correlation within about 2% to about 15% relative to the actual technical variation of the experimentally determined miRNA expression data. In another aspect, the method may further include comparing the miRNA expression in the sample obtained from a patient suspected of having SLE with that in the body fluid sample obtained from a healthy individual.

Diagnosis of SLE

A subject may be identified as having SLE according to diagnostic parameters well known in the art and can have a good or poor prognosis according to diagnostic and/or clinical parameters that are also known in the art. Prognosis may include prediction of overall survival, improvement or maintaining scores (SLEDAI, SLAM, BILAG, etc.), reduction of drugs, such as immunosuppressors, reduction or improvements of comorbidities, such as osteoarthrosis, and/or improvements of quality of life. For example, a subject with SLE who would be identified as a subject as having a good prognosis may be a subject, in whom symptoms are mild or moderate, and/or the subject may be responsive (i.e., shows improvement) to standard treatment protocols, etc. A subject with SLE who would be identified as having a poor prognosis may be a subject, in whom symptoms are severe and/or the subject is minimally or non-responsive (i.e., shows minimal to no improvement) to standard treatment protocols. A correlation can be made between good and poor prognosis and a subject's miRNA markers according to the methods of this present disclosure, which can allow a clinician to determine the most effective treatment regimen for the subject. Thus, a poor prognosis or a good prognosis for SLE would be identified by one of ordinary skill in the art.

Accordingly, an association between the likelihood of a poor prognosis and an increase or a decrease in an amount of one or more miRNAs may be made by detecting an increase or a decrease in an amount of one or more miRNAs in a population of subjects having SLE and a poor prognosis, i.e., subjects in whom symptoms are severe and/or the subjects are minimally or non-responsive (i.e., showing minimal to no improvement) to standard treatment protocols; and associating the detected increase or decrease in the amount of the one or more miRNAs with a poor prognosis in the population of subjects having SLE and a poor prognosis.

Similarly, an association between the likelihood of a poor prognosis and a particular miRNA profile may be made by detecting an increase or a decrease in an amount of one or more miRNAs in a population of subjects having SLE and a poor prognosis, i.e., subjects in whom symptoms are severe and/or the subjects are minimally or non-responsive (i.e., showing minimal to no improvement) to standard treatment protocols; generating the miRNA profile from the detection of the increase or decrease in the amount of the one or more miRNAs; and associating the miRNA profile with a poor prognosis in the population of subjects having SLE and a poor prognosis.

Alternatively, an association between the likelihood of a good prognosis and an increase or a decrease in an amount of one or more miRNAs may be made by detecting an increase or a decrease in an amount one or more miRNAs in a population of patients having SLE and a good prognosis, i.e., subjects in whom symptoms are mild or moderate, and/or the subjects are responsive (i.e., showing improvement) to standard treatment protocols; and associating the detected increase or decrease in the amount of the one or more miRNAs with a good prognosis in the population of subjects having SLE and a good prognosis.

Further, an association between the likelihood of a good prognosis and a particular miRNA profile may be made by detecting an increase or a decrease in an amount of one or more miRNAs in a population of subjects having SLE and a good prognosis, i.e., subjects in whom symptoms are mild or moderate, and/or the subjects are responsive (i.e., show improvement) to standard treatment protocols; generating the miRNA profile from the detection of the increase or decrease in the amount of the one or more miRNAs and associating the miRNA profile with a good prognosis in the population of subjects having SLE and a good prognosis.

An aspect of the present disclosure provides methods of diagnosing SLE using a body fluid sample obtained from a subject, the methods including identifying the patient as having the marker correlated with SLE if detecting an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample or identifying the patient as not having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

In another aspect, the present disclosure provides a method of diagnosing SLE using a body fluid sample obtained from a subject including identifying the patient as having the marker correlated with SLE if detecting an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample or identifying the patient as not having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

In another aspect, the present disclosure provides a method of diagnosing SLE using a body fluid sample obtained from a subject including identifying the patient as having the marker correlated with moderate SLE if detecting a decrease in expression of at least one miRNA selected from SEQ ID NOs: 161-242 and 403-484 compared to a body fluid sample obtained from a healthy individual is detected in the patient sample or identifying the patient as not having the marker correlated with SLE if a decrease in expression of at least one miRNA selected from SEQ ID NOs: 161-242 and 403-484 compared to a body fluid sample obtained from a healthy individual fails to be detected.

In another aspect, the present disclosure provides a method of diagnosing SLE using a body fluid sample obtained from a subject including identifying the patient as having the marker correlated with SLE comorbidity A if detecting an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample or identifying the patient as not having the marker correlated with SLE comorbidity A if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

In another aspect, the present disclosure provides a method of diagnosing SLE using a body fluid sample obtained from a subject including identifying the patient as having the marker correlated with SLE comorbidity B if detecting an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample or identifying the patient as not having the marker correlated with SLE comorbidity B if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

In another aspect, the present disclosure provides a method of diagnosing SLE using a body fluid sample obtained from a subject including identifying the patient as having the marker correlated with SLE comorbidity C if detecting an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample or identifying the patient as not having the marker correlated with SLE comorbidity C if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

In another aspect, the present disclosure provides a method of diagnosing SLE using a body fluid sample obtained from a subject including identifying the patient as having the marker correlated with SLE comorbidity D if detecting an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample or identifying the patient as not having the marker correlated with SLE comorbidity D if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

In other embodiments, the 5′ and/or 3′ end of the miRNA may be truncated. For example, about 1 to about 10 ribonucleotides may be missing from either the 5′ and/or 3′ end of the miRNA.

Pharmaceutical Compositions

miRNAs of the present disclosure and/or their agonists or antagonists thereof may be used directly or in combination with other agents for treating diseases, for example, SLE. The present disclosure may also provide pharmaceutical compositions, which may contain a safe and effective amount of miRNAs of the present disclosure and/or their agonists or antagonists thereof and pharmaceutically acceptable carriers or excipients. Such carriers may comprise (but are not limited to) saline, buffered saline, dextrose, water, glycerol, ethanol, and combinations thereof. Pharmaceutical formulations can be matched to the mode of administration. The pharmaceutical compositions of the present disclosure can be produced as injectable form, such as physiological saline or an aqueous solution containing glucose and other auxiliary agents prepared by conventional methods. Pharmaceutical compositions, such as injectable compositions and solution, may be manufactured under sterile conditions. Therapeutically effective amount of pharmaceutical compositions may be the effective amount of active ingredient of pharmaceutical compositions administered, for example, from about 0.1 μg/kg of body weight to about 10 mg/kg of body weight.

miRNAs of the present disclosure and/or their agonists or antagonists thereof in pharmaceutical compositions may be administered to subjects, e.g., SLE patients, in a safe and effective amount at least about 0.1 μg/kg of body weight, and in most cases not more than about 10 mg/kg of body weight, preferably from about 0.1 μg/kg body weight to about 100 μg/kg of body weight. Particular dosages may be determined based on the route of administration and patient's conditions, all of which are within the skill of the physician of skill.

Treatment

Examples of treatment regimens for SLE are known in the art and may comprise, but are not limited to, administration of nonsteroidal anti-inflammatory drugs (NSAIDs), hydroxychloroquine, corticosteroids, immunosuppressive drugs, such as azathioprine, methotrexate, cyclosporine, mycophenolate mofetil, cyclophosphamide, and tacrolimus, and biological agents, such as belimumab, rituximab, TNF alpha inhibitors, and interferon inhibitors.

Patients who respond well to particular treatment protocols can be analyzed for a specific miRNA profile (e.g., an increase or decrease in an amount of one or more miRNAs associate with SLE) and a correlation can be established according to the methods provided herein. Alternatively, patients who respond poorly to a particular treatment regimen can also be analyzed for a particular miRNA profile (e.g., an increase or decrease in an amount of one or more miRNAs associate with SLE) correlated with the poor response. Then, a subject who is a candidate for treatment for SLE can be assessed for the presence of the appropriate miRNA profile and the most appropriate treatment regimen can be provided.

Accordingly, an association between an effective treatment regimen and an increase or a decrease in an amount of one or more miRNAs is made by detecting an increase or a decrease in an amount of one or more miRNAs in a population of subjects having SLE and for whom an effective treatment regimen for SLE has been identified; and associating the detected increase or decrease in the amount of the one or more miRNAs with an effective treatment regimen for SLE.

Similarly, an association between an effective treatment regimen and a particular miRNA profile may be made by detecting an increase or a decrease in an amount of one or more miRNAs in a population of subjects having SLE and for whom an effective treatment regimen for SLE has been identified; generating the miRNA profile from the detection of the increase or decrease in the amount of the one or more miRNAs; and associating the generated miRNA profile with an effective treatment regimen for SLE.

In some embodiments, the methods of correlating a miRNA profile with treatment regimens can be carried out using a computer database. Thus, the present disclosure may provide a computer-assisted method of identifying a proposed treatment for SLE.

The method may involve the steps of
(a) storing a database of biological data for a plurality of patients, the biological data that is being stored including for each of said plurality of patients (i) a treatment type, (ii) at least one miRNA, an increase or decrease in the amount of which is associated with SLE and (iii) at least one disease progression measure for SLE from which treatment efficacy can be determined; and then
(b) querying the database to determine the dependence on said increase or decrease in the amount of the at least one miRNA of the effectiveness of a treatment type intreating SLE, to thereby identify a proposed treatment as an effective treatment for a subject having a miRNA profile correlated with SLE.

In one embodiment, treatment information for a patient may be entered into the database (through any suitable means such as a window or text interface), miRNA information (e.g., an miRNA profile) for that patient is entered into the database, and disease progression information is entered into the database. These steps may be then repeated until the desired number of patients has been entered into the database. The database can then be queried to determine whether a particular treatment is effective for patients having a particular miRNA profile, not effective for patients having a particular miRNA profile, etc. Such querying can be carried out prospectively or retrospectively on the database by any suitable means, but is generally done by statistical analysis in accordance with known techniques, as described herein.

Small ribonucleic acids, e.g., miRNAs, or their agonists or antagonists may be formulated in nontoxic, inert and pharmaceutically-acceptable aqueous carrier media, in which pH may be at about 5-8, preferably pH at about 6-8, although pH may vary depending on the properties of small ribonucleic acids, e.g., miRNAs, or their agonists or antagonists and may also vary due to changes of diseased conditions to be treated. The pharmaceutical compositions may be administered through conventional routes, including (but not limited to) intramuscular, intravenous, or subcutaneous administration.

Administering miRNAs of the present disclosure and/or their agonists or antagonists thereof to subjects, e.g., SLE patients, may result in increased amount and/or increased expression of miRNAs, thereby preventing or treating diseases associated with reduced amount and/or reduced expression of such miRNAs, e.g., aberrantly activated interferon pathway-related diseases, which may play a crucial role in the pathogenesis of SLE.

In an aspect, the present disclosure provides method of treating SLE including administering to a SLE patients a composition containing antagonists of one or more miRNAs consisting of the nucleotide sequence selected from the group consisting of SEQ ID NO: 1-160 and 243-402, e.g., one or more SEQ ID NO: 1-160 and 243-402antisense molecules, or administering to a SLE patients a composition containing agonists of one or more miRNAs consisting of the nucleotide sequence selected from the group of consisting of SEQ ID NO: 161-242 and 403-484, e.g., one or more SEQ ID NO: 161-242 and 403-484 molecules.

In another aspect, the present disclosure provides method of treating moderate SLE including administering to a SLE patients a composition containing antisense molecules of one or more miRNAs consisting of the nucleotide sequence selected from the group consisting of SEQ ID NO: 1-160 and 243-402 and/or a composition containing one or more miRNAs consisting of the nucleotide sequence selected from the group consisting of SEQ ID NO: 161-242 and 403-484.

Kits

It is further contemplated that the present disclosure may provide kits for use in screening, diagnosing and identifying subjects with SLE. Kits may contain the pharmaceutical compositions of this disclosure, e.g., miRNAs (SEQ ID NO: 1-484). It would be well understood by one of ordinary skill in the art that the kit of this disclosure can comprise one or more containers and/or receptacles to hold the reagents (e.g., nucleic acids, and the like) of the kit, along with appropriate buffers and/or diluents and/or other solutions and directions for using the kit, as would be well known in the art. Such kits can further comprise adjuvants and/or other immunostimulatory or immunomodulating agents, as are well known in the art.

### EXAMPLES

### Example 1: In Situ Extraction of Urinary EV-Included miRNA Using Nanowire-Incorporated Microfluidic Devices

Using a microarray analysis of miRNAs as described herein, specific miRNAs have been demonstrated to be differentially expressed in SLE peripheral blood mononuclear cells (PBMCs) as compared with age and sex matched, healthy normal controls. A stringent criteria of three fold differential miRNA expression levels between SLE and healthy samples was used to identify unique patterns of altered miRNA expression. Such patterns provide complex fingerprints that can serve as molecular biomarkers for SLE diagnosis, prognosis, and/or prediction of therapeutic responses.

TABLE 1

| | SLE patients (n = 30) | Healthy donors (n = 30) |
|---|---|---|
| Age (std) | 44.8 (13.9) | 45.9 (14.9) |
| Sex | | |
| Male | 4 (13.3%) | 4 (13.3%) |
| Female | 26 (86.7%) | 26 (86.7%) |
| Ethnicity | | |
| African American | 11 (36.7%) | 4 (13.3%) |
| Hispanic | 8 (26.7%) | 10 (33.3%) |
| NA | 11 (36.7%) | 16 (53.3%) |
| Severity | | |
| Mild | 12 (40%) | |
| Moderate | 5 (16.7%) | |
| NA | 13 43.3%) | |

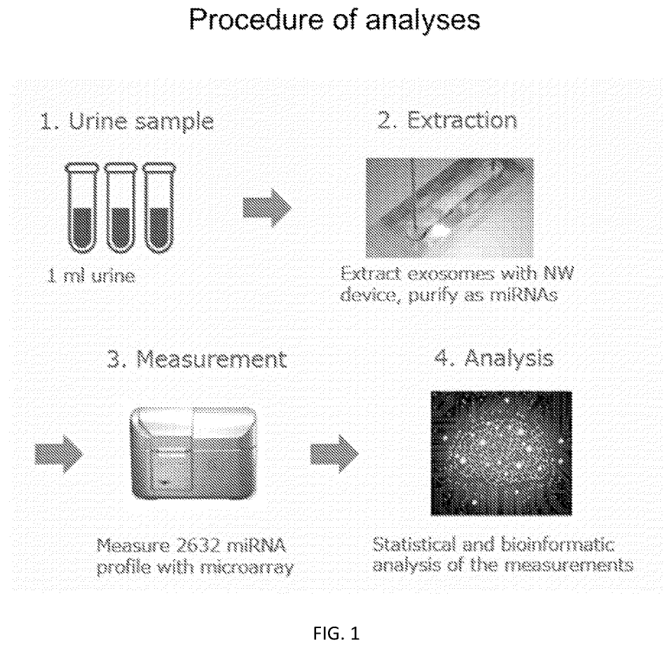

Urine samples obtained from SLE patients and healthy individuals shown in Table 1 were centrifuged (15 mm, 4° C., 3000 g) prior to use to remove apoptotic bodies. Thereafter, 1 ml urine samples were introduced into the nanowire incorporated devices using a syringe pump (KDS-200, KD Scientific Inc.) at a flow rate of 50 μl/min Extractions of miRNA from EVs collected on nanowires were performed by introducing cytolysis buffer M [20 mM tris-HCl (pH 7.4), 200 mM sodium chloride, 2.5 mM magnesium chloride, and 0.05 w/v % NP-40; (Wako Pure Chemical Industries Ltd.) into nanowire incorporated devices using a syringe pump at a flow rate of 50 μl/min (FIG. 1)

Microarray Analyses of miRNA Expression

miRNA expression profiles were obtained using Toray 3D-Gene (Toray Industries) human miRNA chips. miRNA extracted with lysis buffer was purified using SeraMir Exosome RNA Purification Column Kit (System Biosciences Inc.) according to the manufacturer's instructions. 15 μl of purified miRNA was analyzed for 2,632 miRNA profiling using 3D-Gene Human miRNA Oligo chip ver. 21 (Toray Industries). In microarray analyses of miRNA expression, each of the signal intensities corresponds to one species of miRNA. The expression level of each miRNA is expressed as the signal intensities of all miRNA in each microarray, subtracted by the background. Scatter plots were generated for intensities standardized throughout and are shown for intensity equal to or greater than 10. Thus, each point on the scatter plot is a standardized intensity. Signal intensities were log 2 transformed. For comparisons of miRNA between SLE patient and healthy donor urine samples, normalized intensities were log 2 transformed throughout the samples. (FIG. 1)

Identification of Urinary miRNAs as Biomarkers of SLE

The 95% confidence interval was calculated using (mean)±1.96×(mean×CV/100) according to a Z-score of 1.96 (95% confidence level and 5% significance level) and the relation of variability (CVs) (without specific values) to log 2 (strength) provided by Toray. Using X % for CVs in relation to log 2 (strength)=3, the upper limit of the confidence interval was 8+0.16X. The CV values at log 2 (strength)=5 or 6 were 0.7X % and 0.5X % according to the relation. Considering the 5% significance level, CVs for each case were less than 40 and 71%.

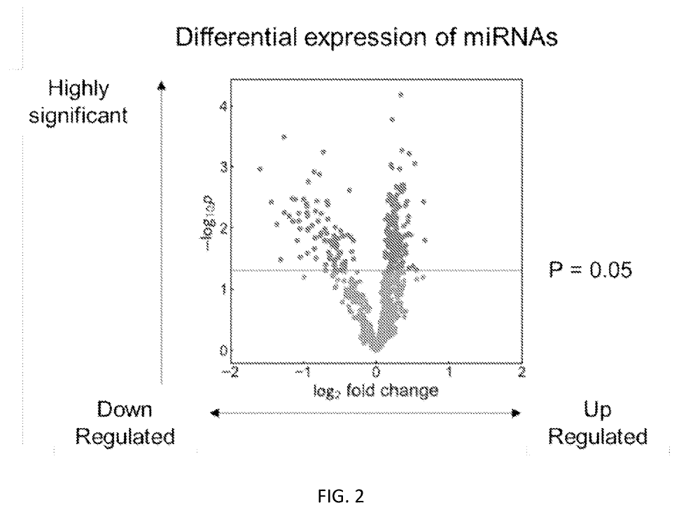

FIG. 2 is a volcano plot that shows 242 miRNAs were differentially expressed, in which 160 miRNAs (Table 2) were significantly up-regulated and 82 miRNAs (Table 3) were significantly down-regulated in SLE patients as compared with that in the healthy individuals. (p<0.05 in t-test). The 82 down-regulated miRNAs among the cohorts appear to have larger fold changes than the 160 miRNAs up-regulated miRNAs. These 242 miRNAs represent biomarker candidates of SLE.

TABLE 2
160 up-regulated miRNAs associated with SLE

| | SEQ | | SEQ | |
|---|---|---|---|---|
| | ID | Mature | ID | Precursor |
| miRNA | NO: | Sequence | NO: | Sequence |
| hsa-miR-365a- | 1 | AGGGACUUU | 243 | ACCGCAGGG |
| 3p | | UGGGGGCAG | | AAAAUGAGG |
| | | AUGUG | | GACUUUUGG |
| | | | | GGGCAGAUG |
| | | | | UGUUUCCAU |
| | | | | UCCACUAUC |
| | | | | AUAAUGCCC |
| | | | | CUAAAAAUC |
| | | | | CUUAUUGCU |
| | | | | CUUGCA |
| hsa-miR-365b- | 2 | AGGGACUUU | 244 | AGAGUGUUC |
| 3p | | CAGGGGCAG | | AAGGACAGC |
| | | CUGU | | AAGAAAAAU |
| | | | | GAGGGACUU |
| | | | | UCAGGGGCA |
| | | | | GCUGUGUUU |
| | | | | UCUGACUCA |
| | | | | GUCAUAAUG |
| | | | | CCCCUAAAA |
| | | | | AUCCUUAUU |
| | | | | GUUCUUGCA |
| | | | | GUGUGCAUC |
| | | | | GGG |
| hsa-let-7b-3p | 3 | CUAUACAAC | 245 | CGGGGUGAG |
| | | CUACUGCCU | | GUAGUAGGU |
| | | UCCC | | UGUGUGGUU |
| | | | | UCAGGGCAG |
| | | | | UGAUGUUGC |
| | | | | CCCUCGGAA |
| | | | | GAUAACUAU |
| | | | | ACAACCUAC |
| | | | | UGCCUUCCC |
| | | | | UG |
| hsa-let-7f-1-3p | 4 | CUAUACAAU | 246 | UCAGAGUGA |
| | | CUAUUGCCU | | GGUAGUAGA |
| | | UCCC | | UUGUAUAGU |
| | | | | UGUGGGGUA |
| | | | | GUGAUUUUA |
| | | | | CCCUGUUCA |
| | | | | GGAGAUAAC |
| | | | | UAUACAAUC |
| | | | | UAUUGCCUU |
| | | | | CCCUGA |
| hsa-miR-1182 | 5 | GAGGGUCUU | 247 | GGGACUUGU |
| | | GGGAGGGAU | | CACUGCCUG |
| | | GUGAC | | UCUCCUCCC |
| | | | | UCUCCAGCA |
| | | | | GCGACUGGA |
| | | | | UUCUGGAGU |
| | | | | CCAUCUAGA |
| | | | | GGGUCUUGG |
| | | | | GAGGGAUGU |
| | | | | GACUGUUGG |
| | | | | GAAGCCC |
| hsa-miR-1185- | 6 | AUAUACAGG | 248 | UUUGGUACU |
| 1-3p | | GGGAGACUC | | UGAAGAGAG |
| | | UUAU | | GAUACCCUU |
| | | | | UGUAUGUUC |
| | | | | ACUUGAUUA |
| | | | | AUGGCGAAU |
| | | | | AUACAGGGG |
| | | | | GAGACUCUU |
| | | | | AUUUGCGUA |
| | | | | UCAAA |
| hsa-miR-1185- | 7 | AUAUACAGG | 249 | UUUGGUACU |
| 2-3p | | GGGAGACUC | | UAAAGAGAG |
| | | UCAU | | GAUACCCUU |
| | | | | UGUAUGUUC |
| | | | | ACUUGAUUA |
| | | | | AUGGCGAAU |
| | | | | AUACAGGGG |
| | | | | GAGACUCUC |
| | | | | AUUUGCGUA |
| | | | | UCAAA |
| hsa-miR-1207- | 8 | UGGCAGGGA | 250 | GCAGGGCUG |
| 5p | | GGCUGGGAG | | GCAGGGAGG |
| | | GGG | | CUGGGAGGG |
| | | | | GCUGGCUGG |
| | | | | GUCUGGUAG |
| | | | | UGGGCAUCA |
| | | | | GCUGGCCCU |
| | | | | CAUUUCUUA |
| | | | | AGACAGCAC |
| | | | | UUCUGU |
| hsa-miR-1224- | 9 | CCCCACCUC | 251 | GUGAGGACU |
| 3p | | CUCUCUCCU | | CGGGAGGUG |
| | | CAG | | GAGGGUGGU |
| | | | | GCCGCCGGG |
| | | | | GCCGGGCGC |
| | | | | UGUUUCAGC |
| | | | | UCGCUUCUC |
| | | | | CCCCCACCU |
| | | | | CCUCUCUCC |
| | | | | UCAG |
| hsa-miR-1225- | 10 | UGAGCCCCU | 252 | GUGGGUACG |
| 3p | | GUGCCGCCC | | GCCCAGUGG |
| | | CCAG | | GGGGGAGAG |
| | | | | GGACACGCC |
| | | | | CUGGGCUCU |
| | | | | GCCCAGGGU |
| | | | | GCAGCCGGA |
| | | | | CUGACUGAG |
| | | | | CCCCUGUGC |
| | | | | CGCCCCCAG |
| hsa-miR-1225- | 11 | GUGGGUACG | 253 | GUGGGUACG |
| 5p | | GCCCAGUGG | | GCCCAGUGG |
| | | GGGG | | GGGGGAGAG |
| | | | | GGACACGCC |
| | | | | CUGGGCUCU |
| | | | | GCCCAGGGU |
| | | | | GCAGCCGGA |
| | | | | CUGACUGAG |
| | | | | CCCCUGUGC |
| | | | | CGCCCCCAG |
| hsa-miR-1227- | 12 | CGUGCCACC | 254 | GUGGGGCCA |
| 3p | | CUUUUCCCC | | GGCGGUGGU |
| | | AG | | GGGCACUGC |
| | | | | UGGGGUGGG |
| | | | | CACAGCAGC |
| | | | | CAUGCAGAG |
| | | | | CGGGCAUUU |
| | | | | GACCCCGUG |
| | | | | CCACCCUUU |
| | | | | UCCCCAG |
| hsa-miR-1228- | 13 | UCACACCUG | 255 | GUGGGCGGG |
| 3p | | CCUCGCCCC | | GGCAGGUGU |
| | | CC | | GUGGUGGGU |
| | | | | GGUGGCCUG |
| | | | | CGGUGAGCA |
| | | | | GGGCCCUCA |
| | | | | CACCUGCCU |
| | | | | CGCCCCCCA |
| | | | | G |
| hsa-miR-1233- | 14 | AGUGGGAGG | 256 | GUGAGUGGG |
| 5p | | CCAGGGCAC | | AGGCCAGGG |
| | | GGCA | | CACGGCAGG |
| | | | | GGGAGCUGC |
| | | | | AGGGCUAUG |
| | | | | GGAGGGGCC |
| | | | | CCAGCGUCU |
| | | | | GAGCCCUGU |
| | | | | CCUCCCGCA |
| | | | | G |
| hsa-miR-1234- | 15 | UCGGCCUGA | 257 | GUGAGUGUG |
| 3p | | CCACCCACC | | GGGUGGCUG |
| | | CCAC | | GGGCGGGG |
| | | | | GGGGCCCGG |
| | | | | GGACGGCUU |
| | | | | GGGCCUGCC |
| | | | | UAGUCGGCC |
| | | | | UGACCACCC |
| | | | | ACCCCACAG |
| hsa-miR-1237- | 16 | UCCUUCUGC | 258 | GUGGGAGGG |
| 3p | | UCCGUCCCC | | CCCAGGCGC |
| | | CAG | | GGGCAGGGG |
| | | | | UGGGGGUGG |
| | | | | CAGAGCGCU |
| | | | | GUCCCGGGG |
| | | | | GCGGGGCCG |
| | | | | AAGCGCGGC |
| | | | | GACCGUAAC |
| | | | | UCCUUCUGC |
| | | | | UCCGUCCCC |
| | | | | CAG |
| hsa-miR-1238- | 17 | CUUCCUCGU | 259 | GUGAGUGGG |
| 3p | | CUGUCUGCC | | AGCCCCAGU |
| | | CC | | GUGUGGUUG |
| | | | | GGGCCAUGG |
| | | | | CGGGUGGGC |
| | | | | AGCCCAGCC |
| | | | | UCUGAGCCU |
| | | | | UCCUCGUCU |
| | | | | GUCUGCCCC |
| | | | | AG |
| hsa-miR-1247- | 18 | ACCCGUCCC | 260 | CCGCUUGCC |
| 5p | | GUUCGUCCC | | UCGCCCAGC |
| | | CGGA | | GCAGCCCCG |
| | | | | GCCGCUGGG |
| | | | | CGCACCCGU |
| | | | | CCCGUUCGU |
| | | | | CCCCGGACG |
| | | | | UUGCUCUCU |
| | | | | ACCCCGGGA |
| | | | | ACGUCGAGA |
| | | | | CUGGAGCGC |
| | | | | CCGAACUGA |
| | | | | GCCACCUUC |
| | | | | GCGGACCCC |
| | | | | GAGAGCGGC |
| | | | | G |
| hsa-miR-125a- | 19 | ACAGGUGAG | 261 | UGCCAGUCU |
| 3p | | GUUCUUGGG | | CUAGGUCCC |
| | | AGCC | | UGAGACCCU |
| | | | | UUAACCUGU |
| | | | | GAGGACAUC |
| | | | | CAGGGUCAC |
| | | | | AGGUGAGGU |
| | | | | UCUUGGGAG |
| | | | | CCUGGCGUC |
| | | | | UGGCC |
| hsa-miR-1267 | 20 | CCUGUUGAA | 262 | CUCCCAAAU |
| | | GUGUAAUCC | | CUCCUGUUG |
| | | CCA | | AAGUGUAAU |
| | | | | CCCCACCUC |
| | | | | CAGCAUUGG |
| | | | | GGAUUACAU |
| | | | | UUCAACAUG |
| | | | | AGAUUUGGA |
| | | | | UGAGGA |
| hsa-miR-1275 | 21 | GUGGGGGAG | 263 | CCUCUGUGA |
| | | AGGCUGUC | | GAAAGGGUG |
| | | | | UGGGGGAGA |
| | | | | GGCUGUCUU |
| | | | | GUGUCUGUA |
| | | | | AGUAUGCCA |
| | | | | AACUUAUUU |
| | | | | UCCCCAAGG |
| | | | | CAGAGGGA |
| hsa-miR-129- | 22 | AAGCCCUUA | 264 | GGAUCUUUU |
| 1-3p | | CCCCAAAAA | | UGCGGUCUG |
| | | GUAU | | GGCUUGCUG |
| | | | | UUCCUCUCA |
| | | | | ACAGUAGUC |
| | | | | AGGAAGCCC |
| | | | | UUACCCCAA |
| | | | | AAAGUAUCU |
| hsa-miR-129- | 23 | AAGCCCUUA | 265 | UGCCCUUCG |
| 2-3p | | CCCCAAAAA | | CGAAUCUUU |
| | | GCAU | | UUGCGGUCU |
| | | | | GGGCUUGCU |
| | | | | GUACAUAAC |
| | | | | UCAAUAGCC |
| | | | | GGAAGCCCU |
| | | | | UACCCCAAA |
| | | | | AAGCAUUUG |
| | | | | CGGAGGGCG |
| hsa-miR-1304- | 24 | UCUCACUGU | 266 | AAACACUUG |
| 3p | | AGCCUCGAA | | AGCCCAGCG |
| | | CCCC | | GUUUGAGGC |
| | | | | UACAGUGAG |
| | | | | AUGUGAUCC |
| | | | | UGCCACAUC |
| | | | | UCACUGUAG |
| | | | | CCUCGAACC |
| | | | | CCUGGGCUC |
| | | | | AAGUGAUUC |
| | | | | A |
| hsa-miR-1323 | 25 | UCAAAACUG | 267 | ACUGAGGUC |
| | | AGGGGCAUU | | CUCAAAACU |
| | | UUCU | | GAGGGGCAU |
| | | | | UUUCUGUGG |
| | | | | UUUGAAAGG |
| | | | | AAAGUGCAC |
| | | | | CCAGUUUUG |
| | | | | GGGAUGUCA |
| | | | | A |
| hsa-miR-133a- | 26 | UUUGGUCCC | 268 | ACAAUGCUU |
| 3p | | CUUCAACCA | | UGCUAGAGC |
| | | GCUG | | UGGUAAAAU |
| | | | | GGAACCAAA |
| | | | | UCGCCUCUU |
| | | | | CAAUGGAUU |
| | | | | UGGUCCCCU |
| | | | | UCAACCAGC |
| | | | | UGUAGCUAU |
| | | | | GCAUUGA |
| hsa-miR-133b | 27 | UUUGGUCCC | 269 | CCUCAGAAG |
| | | CUUCAACCA | | AAAGAUGCC |
| | | GCUA | | CCCUGCUCU |
| | | | | GGCUGGUCA |
| | | | | AACGGAACC |
| | | | | AAGUCCGUC |
| | | | | UUCCUGAGA |
| | | | | GGUUUGGUC |
| | | | | CCCUUCAAC |
| | | | | CAGCUACAG |
| | | | | CAGGGCUGG |
| | | | | CAAUGCCCA |
| | | | | GUCCUUGGA |
| | | | | GA |
| hsa-miR-134- | 28 | UGUGACUGG | 270 | CAGGGUGUG |
| 5p | | UUGACCAGA | | UGACUGGUU |
| | | GGGG | | GACCAGAGG |
| | | | | GGCAUGCAC |
| | | | | UGUGUUCAC |
| | | | | CCUGUGGGC |
| | | | | CACCUAGUC |
| | | | | ACCAACCCU |
| | | | | C |
| hsa-miR-18b- | 29 | UGCCCUAAA | 271 | UGUGUUAAG |
| 3p | | UGCCCCUUC | | GUGCAUCUA |
| | | UGGC | | GUGCAGUUA |
| | | | | GUGAAGCAG |
| | | | | CUUAGAAUC |
| | | | | UACUGCCCU |
| | | | | AAAUGCCCC |
| | | | | UUCUGGCA |
| hsa-miR-191- | 30 | GCUGCGCUU | 272 | CGGCUGGAC |
| 3p | | GGAUUUCGU | | AGCGGGCAA |
| | | CCCC | | CGGAAUCCC |
| | | | | AAAAGCAGC |
| | | | | UGUUGUCUC |
| | | | | CAGAGCAUU |
| | | | | CCAGCUGCG |
| | | | | CUUGGAUUU |
| | | | | CGUCCCCUG |
| | | | | CUCUCCUGC |
| | | | | CU |
| hsa-miR-199a- | 31 | CCCAGUGUU | 273 | GCCAACCCA |
| 5p | | CAGACUACC | | GUGUUCAGA |
| | | UGUUC | | CUACCUGUU |
| | | | | CAGGAGGCU |
| | | | | CUCAAUGUG |
| | | | | UACAGUAGU |
| | | | | CUGCACAUU |
| | | | | GGUUAGGC |
| hsa-miR-199b- | 32 | CCCAGUGUU | 274 | CCAGAGGAC |
| 5p | | UAGACUAUC | | ACCUCCACU |
| | | UGUUC | | CCGUCUACC |
| | | | | CAGUGUUUA |
| | | | | GACUAUCUG |
| | | | | UUCAGGACU |
| | | | | CCCAAAUUG |
| | | | | UACAGUAGU |
| | | | | CUGCACAUU |
| | | | | GGUUAGGCU |
| | | | | GGGCUGGGU |
| | | | | UAGACCCUC |
| | | | | GG |
| hsa-miR-210- | 33 | AGCCCCUGC | 275 | ACCCGGCAG |
| 5p | | CCACCGCAC | | UGCCUCCAG |
| | | ACUG | | GCGCAGGGC |
| | | | | AGCCCCUGC |
| | | | | CCACCGCAC |
| | | | | ACUGCGCUG |
| | | | | CCCCAGACC |
| | | | | CACUGUGCG |
| | | | | UGUGACAGC |
| | | | | GGCUGAUCU |
| | | | | GUGCCUGGG |
| | | | | CAGCGCGAC |
| | | | | CC |
| hsa-miR-2116- | 34 | CCUCCCAUG | 276 | GACCUAGGC |
| 3p | | CCAAGAACU | | UAGGGGUUC |
| | | CCC | | UUAGCAUAG |
| | | | | GAGGUCUUC |
| | | | | CCAUGCUAA |
| | | | | GAAGUCCUC |
| | | | | CCAUGCCAA |
| | | | | GAACUCCCA |
| | | | | GACUAGGA |
| hsa-miR-216b- | 35 | ACACACUUA | 277 | GCAGACUGG |
| 3p | | CCCGUAGAG | | AAAAUCUCU |
| | | AUUCUA | | GCAGGCAAA |
| | | | | UGUGAUGUC |
| | | | | ACUGAGGAA |
| | | | | AUCACACAC |
| | | | | UUACCCGUA |
| | | | | GAGAUUCUA |
| | | | | CAGUCUGAC |
| | | | | A |
| hsa-miR-223- | 36 | UGUCAGUUU | 278 | CCUGGCCUC |
| 3p | | GUCAAAUAC | | CUGCAGUGC |
| | | CCCA | | CACGCUCCG |
| | | | | UGUAUUUGA |
| | | | | CAAGCUGAG |
| | | | | UUGGACACU |
| | | | | CCAUGUGGU |
| | | | | AGAGUGUCA |
| | | | | GUUUGUCAA |
| | | | | AUACCCCAA |
| | | | | GUGCGGCAC |
| | | | | AUGCUUACC |
| | | | | AG |
| hsa-miR-296- | 37 | AGGGCCCCC | 279 | AGGACCCUU |
| 5p | | CCUCAAUCC | | CCAGAGGGC |
| | | UGU | | CCCCCCUCA |
| | | | | AUCCUGUUG |
| | | | | UGCCUAAUU |
| | | | | CAGAGGGUU |
| | | | | GGGUGGAGG |
| | | | | CUCUCCUGA |
| | | | | AGGGCUCU |
| hsa-miR-3085- | 38 | UCUGGCUGC | 280 | CCCUACUCU |
| 3p | | UAUGGCCCC | | GGGAAGGUG |
| | | CUC | | CCAUUCUGA |
| | | | | GGGCCAGGA |
| | | | | GUUUGAUUA |
| | | | | UGUGUCACU |
| | | | | CUGGCUGCU |
| | | | | AUGGCCCCC |
| | | | | UCCCAGGGU |
| | | | | CUGG |
| hsa-miR- | 39 | CAACCUCGA | 281 | GAGGGAAAG |
| 3150b-5p | | GGAUCUCCC | | CAGGCCAAC |
| | | CAGC | | CUCGAGGAU |
| | | | | CUCCCCAGC |
| | | | | CUUGGCGUU |
| | | | | CAGGUGCUG |
| | | | | AGGAGAUCG |
| | | | | UCGAGGUUG |
| | | | | GCCUGCUUC |
| | | | | CCCUC |
| hsa-miR-3162- | 40 | UCCCUACCC | 282 | CUGACUUUU |
| 3p | | CUCCACUCC | | UUAGGGAGU |
| | | CCA | | AGAAGGGUG |
| | | | | GGGAGCAUG |
| | | | | AACAAUGUU |
| | | | | UCUCACUCC |
| | | | | CUACCCCUC |
| | | | | CACUCCCCA |
| | | | | AAAAAGUCA |
| | | | | G |
| hsa-miR-3189- | 41 | UGCCCCAUC | 283 | GCCUCAGUU |
| 5p | | UGUGCCCUG | | GCCCCAUCU |
| | | GGUAGGA | | GUGCCCUGG |
| | | | | GUAGGAAUA |
| | | | | UCCUGGAUC |
| | | | | CCCUUGGGU |
| | | | | CUGAUGGGG |
| | | | | UAGCCGAUG |
| | | | | C |
| hsa-miR-3190- | 42 | UCUGGCCAG | 284 | CUGGGGUCA |
| 5p | | CUACGUCCC | | CCUGUCUGG |
| | | CA | | CCAGCUACG |
| | | | | UCCCCACGG |
| | | | | CCCUUGUCA |
| | | | | GUGUGGAAG |
| | | | | GUAGACGGC |
| | | | | CAGAGAGGU |
| | | | | GACCCCGG |
| hsa-miR-328- | 43 | GGGGGGGCA | 285 | UGGAGUGGG |
| 5p | | GGAGGGGCU | | GGGGCAGGA |
| | | CAGGG | | GGGGCUCAG |
| | | | | GGAGAAAGU |
| | | | | GCAUACAGC |
| | | | | CCCUGGCCC |
| | | | | UCUCUGCCC |
| | | | | UUCCGUCCC |
| | | | | CUG |
| hsa-miR-331- | 44 | GCCCCUGGG | 286 | GAGUUUGGU |
| 3p | | CCUAUCCUA | | UUUGUUUGG |
| | | GAA | | GUUUGUUCU |
| | | | | AGGUAUGGU |
| | | | | CCCAGGGAU |
| | | | | CCCAGAUCA |
| | | | | AACCAGGCC |
| | | | | CCUGGGCCU |
| | | | | AUCCUAGAA |
| | | | | CCAACCUAA |
| | | | | GCUC |
| hsa-miR-361- | 45 | UCCCCCAGG | 287 | GGAGCUUAU |
| 3p | | UGUGAUUCU | | CAGAAUCUC |
| | | GAUUU | | CAGGGGUAC |
| | | | | UUUAUAAUU |
| | | | | UCAAAAAGU |
| | | | | CCCCCAGGU |
| | | | | GUGAUUCUG |
| | | | | AUUUGCUUC |
| hsa-miR-3614- | 46 | CCACUUGGA | 288 | GGUUCUGUC |
| 5p | | UCUGAAGGC | | UUGGGCCAC |
| | | UGCCC | | UUGGAUCUG |
| | | | | AAGGCUGCC |
| | | | | CCUUUGCUC |
| | | | | UCUGGGGUA |
| | | | | GCCUUCAGA |
| | | | | UCUUGGUGU |
| | | | | UUUGAAUUC |
| | | | | UUACU |
| hsa-miR-365a- | 47 | AGGGACUUU | 289 | ACCGCAGGG |
| 5p | | UGGGGGCAG | | AAAAUGAGG |
| | | AUGUG | | GACUUUUGG |
| | | | | GGGCAGAUG |
| | | | | UGUUUCCAU |
| | | | | UCCACUAUC |
| | | | | AUAAUGCCC |
| | | | | CUAAAAAUC |
| | | | | CUUAUUGCU |
| | | | | CUUGCA |
| hsa-miR-3714 | 48 | GAAGGCAGC | 290 | GAAGGCAGC |
| | | AGUGCUCCC | | AGUGCUCCO |
| | | CUGU | | CUGUGACGU |
| | | | | GCUCCAUCA |
| | | | | CCGGGCAGG |
| | | | | GAAGACACC |
| | | | | GCUGCCACC |
| | | | | UC |
| hsa-miR-371a- | 49 | ACUCAAACU | 291 | GUGGCACUC |
| 5p | | GUGGGGGCA | | AAACUGUGG |
| | | CU | | GGGCACUUU |
| | | | | CUGCUCUCU |
| | | | | GGUGAAAGU |
| | | | | GCCGCCAUC |
| | | | | UUUUGAGUG |
| | | | | UUAC |
| hsa-miR-371b- | 50 | AAGUGCCCC | 292 | GGUAACACU |
| 3p | | CACAGUUUG | | CAAAAGAUG |
| | | AGUGC | | GCGGCACUU |
| | | | | UCACCAGAG |
| | | | | AGCAGAAAG |
| | | | | UGCCCCCAC |
| | | | | AGUUUGAGU |
| | | | | GCC |
| hsa-miR-375- | 51 | GCGACGAGC | 293 | CCCCGCGAC |
| 5p | | CCCUCGCAC | | GAGCCCCUC |
| | | AAACC | | GCACAAACC |
| | | | | GGACCUGAG |
| | | | | CGUUUUGUU |
| | | | | CGUUCGGCU |
| | | | | CGCGUGAGG |
| | | | | C |
| hsa-miR-3943 | 52 | UAGCCCCCA | 294 | CACACAGAC |
| | | GGCUUCACU | | GGCAGCUGC |
| | | UGGCG | | GGCCUAGCC |
| | | | | CCCAGGCUU |
| | | | | CACUUGGCG |
| | | | | UGGACAACU |
| | | | | UGCUAAGUA |
| | | | | AAGUGGGGG |
| | | | | GUGGGCCAC |
| | | | | GGCUGGCUC |
| | | | | CUACCUGGA |
| | | | | C |
| hsa-miR-409- | 53 | GAAUGUUGC | 295 | UGGUACUCG |
| 3p | | UCGGUGAAC | | GGGAGAGGU |
| | | CCCU | | UACCCGAGC |
| | | | | AACUUUGCA |
| | | | | UCUGGACGA |
| | | | | CGAAUGUUG |
| | | | | CUCGGUGAA |
| | | | | CCCCUUUUC |
| | | | | GGUAUCA |
| hsa-miR-4269 | 54 | GCAGGCACA | 296 | ACAGCGCCC |
| | | GACAGCCCU | | UGCAGGCAC |
| | | GGC | | AGACAGCCC |
| | | | | UGGCUUCUG |
| | | | | CCUCUUUCU |
| | | | | UUGUGGAAG |
| | | | | CCACUCUGU |
| | | | | CAGGCCUGG |
| | | | | GAUGGAGGG |
| | | | | GCA |
| hsa-miR-4271 | 55 | GGGGGAAGA | 297 | AAAUCUCUC |
| | | AAAGGUGGG | | UCCAUAUCU |
| | | G | | UUCCUGCAG |
| | | | | CCCCCAGGU |
| | | | | GGGGGGGAA |
| | | | | GAAAAGGUG |
| | | | | GGGAAUUAG |
| | | | | AUUC |
| hsa-miR-4274 | 56 | CAGCAGUCC | 298 | GGGGCAUUU |
| | | CUCCCCCUG | | AGGGUAACU |
| | | | | GAGCUGCUG |
| | | | | CCGGGGCCU |
| | | | | GGCGCUCCU |
| | | | | CUACCUUGU |
| | | | | CAGGUGACC |
| | | | | CAGCAGUCC |
| | | | | CUCCCCCUG |
| | | | | CAUGGUGCC |
| | | | | C |
| hsa-miR-4281 | 57 | GGGUCCCGG | 299 | GCUGGGGGU |
| | | GGAGGGGGG | | CCCCCGACA |
| | | | | GUGUGGAGC |
| | | | | UGGGGCCGG |
| | | | | GUCCCGGGG |
| | | | | AGGGGGGUU |
| | | | | CUGGGCAG |
| hsa-miR-4284 | 58 | GGGCUCACA | 300 | GUUCUGUGA |
| | | UCACCCCAU | | GGGGCUCAC |
| | | | | AUCACCCCA |
| | | | | UCAAAGUGG |
| | | | | GGACUCAUG |
| | | | | GGGAGAGGG |
| | | | | GGUAGUUAG |
| | | | | GAGCUUUGA |
| | | | | UAGAGGCGG |
| hsa-miR-4286 | 59 | ACCCCACUC | 301 | UACUUAUGG |
| | | CUGGUACC | | CACCCCACU |
| | | | | CCUGGUACC |
| | | | | AUAGUCAUA |
| | | | | AGUUAGGAG |
| | | | | AUGUUAGAG |
| | | | | CUGUGAGUA |
| | | | | CCAUGACUU |
| | | | | AAGUGUGGU |
| | | | | GGCUUAAAC |
| | | | | AUG |
| hsa-miR-4307 | 60 | AAUGUUUUU | 302 | UCAGAAGAA |
| | | UCCUGUUUC | | AAAACAGGA |
| | | C | | GAUAAAGUU |
| | | | | UGUGAUAAU |
| | | | | GUUUGUCUA |
| | | | | UAUAGUUAU |
| | | | | GAAUGUUUU |
| | | | | UUCCUGUUU |
| | | | | CCUUCAGGG |
| | | | | CCA |
| hsa-miR-4312 | 61 | GGCCUUGUU | 303 | GAAAGGUUG |
| | | CCUGUCCCC | | GGGGCACAG |
| | | A | | AGAGCAAGG |
| | | | | AGCCUUCCC |
| | | | | CAGAGGAGU |
| | | | | CAGGCCUUG |
| | | | | UUCCUGUCC |
| | | | | CCAUUCCUC |
| | | | | AGAG |
| hsa-miR-4313 | 62 | AGCCCCCUG | 304 | GAUCAGGCC |
| | | GCCCCAAAC | | CAGCCCCCU |
| | | CC | | GGCCCCAAA |
| | | | | CCCUGCAGC |
| | | | | CCCAGCUGG |
| | | | | AGGAUGAGG |
| | | | | AGAUGCUGG |
| | | | | GCUUGGGUG |
| | | | | GGGGAAUCA |
| | | | | GGGGUGUAA |
| | | | | AGGGGCCUG |
| | | | | CU |
| hsa-miR-4323 | 63 | CAGCCCCAC | 305 | CGGGGCCCA |
| | | AGCCUCAGA | | GGCGGGCAU |
| | | | | GUGGGGUGU |
| | | | | CUGGAGACG |
| | | | | CCAGGCAGC |
| | | | | CCCACAGCC |
| | | | | UCAGACCUC |
| | | | | GGGCAC |
| hsa-miR- | 64 | ACAGGAGUG | 306 | CAUCCUCCU |
| 4433a-3p | | GGGGUGGGA | | UACGUCCCA |
| | | CAU | | CCCCCCACU |
| | | | | CCUGUUUCU |
| | | | | GGUGAAAUA |
| | | | | UUCAAACAG |
| | | | | GAGUGGGGG |
| | | | | UGGGACAUA |
| | | | | AGGAGGAUA |
| hsa-miR- | 65 | CGUCCCACC | 307 | CAUCCUCCU |
| 4433a-5p | | CCCCACUCC | | UACGUCCCA |
| | | UGU | | CCCCCCACU |
| | | | | CCUGUUUCU |
| | | | | GGUGAAAUA |
| | | | | UUCAAACAG |
| | | | | GAGUGGGGG |
| | | | | UGGGACAUA |
| | | | | AGGAGGAUA |
| hsa-miR- | 66 | AUGUCCCAC | 308 | UGUGUUCCC |
| 4433b-5p | | CCCCACUCC | | UAUCCUCCU |
| | | UGU | | UAUGUCCCA |
| | | | | CCCCCACUC |
| | | | | CUGUUUGAA |
| | | | | UAUUUCACC |
| | | | | AGAAACAGG |
| | | | | AGUGGGGGG |
| | | | | UGGGACGUA |
| | | | | AGGAGGAUG |
| | | | | GGGGAAAGA |
| | | | | ACA |
| hsa-miR-4447 | 67 | GGUGGGGGC | 309 | GUUCUAGAG |
| | | UGUUGUUU | | CAUGGUUUC |
| | | | | UCAUCAUUU |
| | | | | GCACUACUG |
| | | | | AUACUUGGG |
| | | | | GUCAGAUAA |
| | | | | UUGUUUGUG |
| | | | | GUGGGGGCU |
| | | | | GUUGUUUGC |
| | | | | AUUGUAGGA |
| | | | | U |
| hsa-miR-449b- | 68 | CAGCCACAA | 310 | UGACCUGAA |
| 3p | | CUACCCUGC | | UCAGGUAGG |
| | | CACU | | CAGUGUAUU |
| | | | | GUUAGCUGG |
| | | | | CUGCUUGGG |
| | | | | UCAAGUCAG |
| | | | | CAGCCACAA |
| | | | | CUACCCUGC |
| | | | | CACUUGCUU |
| | | | | CUGGAUAAA |
| | | | | UUCUUCU |
| hsa-miR-4642 | 69 | AUGGCAUCG | 311 | CACAACUGC |
| | | UCCCCUGGU | | AUGGCAUCG |
| | | GGCU | | UCCCCUGGU |
| | | | | GGCUGUGGC |
| | | | | CUAGGGCAA |
| | | | | GCCACAAAG |
| | | | | CCACUCAGU |
| | | | | GAUGAUGCC |
| | | | | AGCAGUUGU |
| | | | | G |
| hsa-miR-4649- | 70 | UCUGAGGCC | 312 | UCUGGGCGA |
| 3p | | UGCCUCUCC | | GGGGUGGGC |
| | | CCA | | UCUCAGAGG |
| | | | | GGCUGGCAG |
| | | | | UACUGCUCU |
| | | | | GAGGCCUGC |
| | | | | CUCUCCCCA |
| | | | | G |
| hsa-miR-4652- | 71 | GUUCUGUUA | 313 | UAUUGGACG |
| 3p | | ACCCAUCCC | | AGGGGACUG |
| | | CUCA | | GUUAAUAGA |
| | | | | ACUAACUAA |
| | | | | CCAGAACUA |
| | | | | UUUUGUUCU |
| | | | | GUUAACCCA |
| | | | | UCCCCUCAU |
| | | | | CUAAUA |
| hsa-miR-4664- | 72 | CUUCCGGUC | 314 | GUUGGGGGC |
| 3p | | UGUGAGCCC | | UGGGGUGCC |
| | | CGUC | | CACUCCGCA |
| | | | | AGUUAUCAC |
| | | | | UGAGCGACU |
| | | | | UCCGGUCUG |
| | | | | UGAGCCCCG |
| | | | | UCCUCCGC |
| hsa-miR-4665- | 73 | CUCGGCCGC | 315 | CUCGAGGUG |
| 3p | | GGCGCGUAG | | CUGGGGGAC |
| | | CCCCCGCC | | GCGUGAGCG |
| | | | | CGAGCCGCU |
| | | | | UCCUCACGG |
| | | | | CUCGGCCGC |
| | | | | GGCGCGUAG |
| | | | | CCCCCGCCA |
| | | | | CAUCGGG |
| hsa-miR-4667- | 74 | ACUGGGGAG | 316 | UGACUGGGG |
| 5p | | CAGAAGGAG | | AGCAGAAGG |
| | | AACC | | AGAACCCAA |
| | | | | GAAAAGCUG |
| | | | | ACUUGGAGG |
| | | | | UCCCUCCUU |
| | | | | CUGUCCCCA |
| | | | | CAG |
| hsa-miR-4687- | 75 | UGGCUGUUG | 317 | ACCUGAGGA |
| 3p | | GAGGGGGCA | | GCCAGCCCU |
| | | GGC | | CCUCCCGCA |
| | | | | CCCAAACUU |
| | | | | GGAGCACUU |
| | | | | GACCUUUGG |
| | | | | CUGUUGGAG |
| | | | | GGGGCAGGC |
| | | | | UCGCGGGU |
| hsa-miR-4689 | 76 | UUGAGGAGA | 318 | GGUUUCUCC |
| | | CAUGGUGGG | | UUGAGGAGA |
| | | GGCC | | CAUGGUGGG |
| | | | | GGCCGGUCA |
| | | | | GGCAGCCCA |
| | | | | UGCCAUGUG |
| | | | | UCCUCAUGG |
| | | | | AGAGGCC |
| hsa-miR-4697- | 77 | UGUCAGUGA | 319 | GGGCCCAGA |
| 3p | | CUCCUGCCC | | AGGGGGCGC |
| | | CUUGGU | | AGUCACUGA |
| | | | | CGUGAAGGG |
| | | | | ACCACAUCC |
| | | | | CGCUUCAUG |
| | | | | UCAGUGACU |
| | | | | CCUGCCCCU |
| | | | | UGGUCU |
| hsa-miR-4709- | 78 | UUGAAGAGG | 320 | CUGCUUCAA |
| 3p | | AGGUGCUCU | | CAACAGUGA |
| | | GUAGC | | CUUGCUCUC |
| | | | | CAAUGGUAU |
| | | | | CCAGUGAUU |
| | | | | CGUUGAAGA |
| | | | | GGAGGUGCU |
| | | | | CUGUAGCAG |
| hsa-miR-4714- | 79 | AACUCUGAC | 321 | AUUUUGGCC |
| 5p | | CCCUUAGGU | | AACUCUGAC |
| | | UGAU | | CCCUUAGGU |
| | | | | UGAUGUCAG |
| | | | | AAUGAGGUG |
| | | | | UACCAACCU |
| | | | | AGGUGGUCA |
| | | | | GAGUUGGCC |
| | | | | AAAAU |
| hsa-miR-4716- | 80 | UCCAUGUUU | 322 | CAUACUUUG |
| 5p | | CCUUCCCCC | | UCUCCAUGU |
| | | UUCU | | UUCCUUCCC |
| | | | | CCUUCUGUA |
| | | | | UACAUGUAU |
| | | | | ACAGGAGGA |
| | | | | AGGGGGAAG |
| | | | | GAAACAUGG |
| | | | | AGACAAAGU |
| | | | | GUG |
| hsa-miR-4717- | 81 | ACACAUGGG | 323 | GGCAGUGUU |
| 3p | | UGGCUGUGG | | UAGGCCACA |
| | | CCU | | GCCACCCAU |
| | | | | GUGUAGGGG |
| | | | | UGGCUACAC |
| | | | | AUGGGUGGC |
| | | | | UGUGGCCUA |
| | | | | AACACUGCC |
| hsa-miR-4728- | 82 | CAUGCUGAC | 324 | GUGGGAGGG |
| 3p | | CUCCCUCCU | | GAGAGGCAG |
| | | GCCCCAG | | CAAGCACAC |
| | | | | AGGGCCUGG |
| | | | | GACUAGCAU |
| | | | | GCUGACCUC |
| | | | | CCUCCUGCC |
| | | | | CCAG |
| hsa-miR-4731- | 83 | CACACAAGU | 325 | CCCUGCCAG |
| 3p | | GGCCCCCAA | | UGCUGGGGG |
| | | CACU | | CCACAUGAG |
| | | | | UGUGCAGUC |
| | | | | AUCCACACA |
| | | | | CAAGUGGCC |
| | | | | CCCAACACU |
| | | | | GGCAGGG |
| hsa-miR-4749- | 84 | CGCCCCUCC | 326 | CCUGCGGGG |
| 3p | | UGCCCCCAC | | ACAGGCCAG |
| | | AG | | GGCAUCUAG |
| | | | | GCUGUGCAC |
| | | | | AGUGACGCC |
| | | | | CCUCCUGCC |
| | | | | CCCACAG |
| hsa-miR-4750- | 85 | CCUGACCCA | 327 | CGCUCGGGC |
| 3p | | CCCCCUCCC | | GGAGGUGGU |
| | | GCAG | | UGAGUGCCG |
| | | | | ACUGGCGCC |
| | | | | UGACCCACC |
| | | | | CCCUCCCGC |
| | | | | AG |
| hsa-miR-4756- | 86 | CAGGGAGGC | 328 | GGGAUAAAA |
| 5p | | GCUCACUCU | | UGCAGGGAG |
| | | CUGCU | | GCGCUCACU |
| | | | | CUCUGCUGC |
| | | | | CGAUUCUGC |
| | | | | ACCAGAGAU |
| | | | | GGUUGCCUU |
| | | | | CCUAUAUUU |
| | | | | UGUGUC |
| hsa-miR-4788 | 87 | UUACGGACC | 329 | AAUGAAGGA |
| | | AGCUAAGGG | | UUACGGACC |
| | | AGGC | | AGCUAAGGG |
| | | | | AGGCAUUAG |
| | | | | GAUCCUUAU |
| | | | | UCUUGCCUC |
| | | | | CCUUAGUUG |
| | | | | GUCCCUAAU |
| | | | | CCUUCGUU |
| hsa-miR-484 | 88 | UCAGGCUCA | 330 | AGCCUCGUC |
| | | GUCCCCUCC | | AGGCUCAGU |
| | | CGAU | | CCCCUCCCG |
| | | | | AUAAACCCC |
| | | | | UAAAUAGGG |
| | | | | ACUUUCCCG |
| | | | | GGGGGUGAC |
| | | | | CCUGGCUUU |
| | | | | UUUGGCG |
| hsa-miR-486- | 89 | UCCUGUACU | 331 | GCAUCCUGU |
| 5p | | GAGCUGCCC | | ACUGAGCUG |
| | | CGAG | | CCCCGAGGC |
| | | | | CCUUCAUGC |
| | | | | UGCCCAGCU |
| | | | | CGGGGCAGC |
| | | | | UCAGUACAG |
| | | | | GAUAC |
| hsa-miR-5010- | 90 | UUUUGUGUC | 332 | GAUCCAGGG |
| 3p | | UCCCAUUCC | | AACCCUAGA |
| | | CCAG | | GCAGGGGGA |
| | | | | UGGCAGAGC |
| | | | | AAAAUUCAU |
| | | | | GGCCUACAG |
| | | | | CUGCCUCUU |
| | | | | GCCAAACUG |
| | | | | CACUGGAUU |
| | | | | UUGUGUCUC |
| | | | | CCAUUCCCC |
| | | | | AGAGCUGUC |
| | | | | UGAGGUGCU |
| | | | | UUG |
| hsa-miR-514b- | 91 | UUCUCAAGA | 333 | CAUGUGGUA |
| 5p | | GGGAGGCAA | | CUCUUCUCA |
| | | UCAU | | AGAGGGAGG |
| | | | | CAAUCAUGU |
| | | | | GUAAUUAGA |
| | | | | UAUGAUUGA |
| | | | | CACCUCUGU |
| | | | | GAGUGGAGU |
| | | | | AACACAUG |
| hsa-miR-518b | 92 | CAAAGCGCU | 334 | UCAUGCUGU |
| | | CCCCUUUAG | | GGCCCUCCA |
| | | AGGU | | GAGGGAAGC |
| | | | | GCUUUCUGU |
| | | | | UGUCUGAAA |
| | | | | GAAAACAAA |
| | | | | GCGCUCCCC |
| | | | | UUUAGAGGU |
| | | | | UUACGGUUU |
| | | | | GA |
| hsa-miR-5195- | 93 | AUCCAGUUC | 335 | GAGCAAAAA |
| 3p | | UCUGAGGGG | | CCAGAGAAC |
| | | GCU | | AACAUGGGA |
| | | | | GCGUUCCUA |
| | | | | ACCCCUAAG |
| | | | | GCAACUGGA |
| | | | | UGGGAGACC |
| | | | | UGACCCAUC |
| | | | | CAGUUCUCU |
| | | | | GAGGGGGCU |
| | | | | CUUGUGUGU |
| | | | | UCUACAAGG |
| | | | | UUGUUCA |
| hsa-miR-5699- | 94 | UGCCCCAAC | 336 | CUGUACCCC |
| 5p | | AAGGAAGGA | | UGCCCCAAC |
| | | CAAG | | AAGGAAGGA |
| | | | | CAAGAGGUG |
| | | | | UGAGCCACA |
| | | | | CACACGCCU |
| | | | | GGCCUCCUG |
| | | | | UCUUUCCUU |
| | | | | GUUGGAGCA |
| | | | | GGGAUGUAG |
| hsa-miR-5739 | 95 | GCGGAGAGA | 337 | GGUUGGCUA |
| | | GAAUGGGGA | | UAACUAUCA |
| | | GC | | UUUCCAAGG |
| | | | | UUGUGCUUU |
| | | | | UAGGAAAUG |
| | | | | UUGGCUGUC |
| | | | | CUGCGGAGA |
| | | | | GAGAAUGGG |
| | | | | GAGCCAGG |
| hsa-miR-6069 | 96 | GGGCUAGGG | 338 | UGGUGACCC |
| | | CCUGCUGCC | | CUGGGCUAG |
| | | CCC | | GGCCUGCUG |
| | | | | CCCCCUGCC |
| | | | | CAGUGCAGG |
| | | | | AGGGUGGAG |
| | | | | GGUCACUCC |
| | | | | UUAGGUGGU |
| | | | | CCCAGUG |
| hsa-miR-625- | 97 | GACUAUAGA | 339 | AGGGUAGAG |
| 3p | | ACUUUCCCC | | GGAUGAGGG |
| | | CUCA | | GGAAAGUUC |
| | | | | UAUAGUCCU |
| | | | | GUAAUUAGA |
| | | | | UCUCAGGAC |
| | | | | UAUAGAACU |
| | | | | UUCCCCCUC |
| | | | | AUCCCUCUG |
| | | | | CCCU |
| hsa-miR-634 | 98 | AACCAGCAC | 340 | AAACCCACA |
| | | CCCAACUUU | | CCACUGCAU |
| | | GGAC | | UUUGGCCAU |
| | | | | CGAGGGUUG |
| | | | | GGGCUUGGU |
| | | | | GUCAUGCCC |
| | | | | CAAGAUAAC |
| | | | | CAGCACCCC |
| | | | | AACUUUGGA |
| | | | | CAGCAUGGA |
| | | | | UUAGUCU |
| hsa-miR-637 | 99 | ACUGGGGGC | 341 | UGGCUAAGG |
| | | UUUCGGGCU | | UGUUGGCUC |
| | | CUGCGU | | GGGCUCCCC |
| | | | | ACUGCAGUU |
| | | | | ACCCUCCCC |
| | | | | UCGGCGUUA |
| | | | | CUGAGCACU |
| | | | | GGGGGCUUU |
| | | | | CGGGCUCUG |
| | | | | CGUCUGCAC |
| | | | | AGAUACUUC |
| hsa-miR-6503- | 100 | AGGUCUGCA | 342 | AAUGGUCCC |
| 5p | | UUCAAAUCC | | CCCAGGGAG |
| | | CCAGA | | GUCUGCAUU |
| | | | | CAAAUCCCC |
| | | | | AGAAGCUGA |
| | | | | GGAUUAGGG |
| | | | | GACUAGGAU |
| | | | | GCAGACCUC |
| | | | | CCUGGGGGA |
| | | | | CCAUU |
| hsa-miR-6507- | 101 | CAAAGUCCU | 343 | GGAGGGAAG |
| 3p | | UCCUAUUUU | | AAUAGGAGG |
| | | UCCC | | GACUUUGUA |
| | | | | UUGUGGUUC |
| | | | | AGUACCAUG |
| | | | | CAAAGUCCU |
| | | | | UCCUAUUUU |
| | | | | UCCCUCC |
| hsa-miR-6728- | 102 | UCUCUGCUC | 344 | CUAGAUUGG |
| 3p | | UGCUCUCCC | | GAUGGUAGG |
| | | CAG | | ACCAGAGGG |
| | | | | GCUUACUGC |
| | | | | CCUGUGGGG |
| | | | | CUCUCUGGA |
| | | | | CCCAGUGCC |
| | | | | AUGCUUCUC |
| | | | | UGCUCUGCU |
| | | | | CUCCCCAG |
| hsa-miR-6731- | 103 | UCUAUUCCC | 345 | ACAGGUGGG |
| 3p | | CACUCUCCO | | AGAGCAGGG |
| | | CAG | | UAUUGUGGA |
| | | | | AGCUCCAGG |
| | | | | UGCCAACCA |
| | | | | CCUGCCUCU |
| | | | | AUUCCCCAC |
| | | | | UCUCCCCAG |
| hsa-miR-6742- | 104 | ACCUGGGUU | 346 | GAGGGAGUG |
| 3p | | GUCCCCUCU | | GGGUGGGAC |
| | | AG | | CCAGCUGUU |
| | | | | GGCCAUGGC |
| | | | | GACAACACC |
| | | | | UGGGUUGUC |
| | | | | CCCUCUAG |
| hsa-miR-6744- | 105 | UGGAUGACA | 347 | UCACGUGGA |
| 5p | | GUGGAGGCC | | UGACAGUGG |
| | | U | | AGGCCUCCU |
| | | | | GGAUCUCUA |
| | | | | GGUCUCAGG |
| | | | | GCCUCUCUU |
| | | | | GUCAUCCUG |
| | | | | CAG |
| hsa-miR-6752- | 106 | UCCCUGCCC | 348 | AUGGAGGGG |
| 3p | | CCAUACUCC | | GGUGUGGAG |
| | | CAG | | CCAGGGGGC |
| | | | | CCAGGUCUA |
| | | | | CAGCUUCUC |
| | | | | CCCGCUCCC |
| | | | | UGCCCCCAU |
| | | | | ACUCCCAG |
| hsa-miR-6756- | 107 | UCCCCUUCC | 349 | ACCCUAGGG |
| 3p | | UCCCUGCCC | | UGGGGCUGG |
| | | AG | | AGGUGGGGC |
| | | | | UGAGGCUGA |
| | | | | GUCUUCCUC |
| | | | | CCCUUCCUC |
| | | | | CCUGCCCAG |
| hsa-miR-6757- | 108 | AACACUGGC | 350 | GGGCUUAGG |
| 3p | | CUUGCUAUC | | GAUGGGAGG |
| | | CCCA | | CCAGGAUGA |
| | | | | AGAUUAAUC |
| | | | | CCUAAUCCC |
| | | | | CAACACUGG |
| | | | | CCUUGCUAU |
| | | | | CCCCAG |
| hsa-miR-6757- | 109 | UAGGGAUGG | 351 | GGGCUUAGG |
| 5p | | GAGGCCAGG | | GAUGGGAGG |
| | | AUGA | | CCAGGAUGA |
| | | | | AGAUUAAUC |
| | | | | CCUAAUCCC |
| | | | | CAACACUGG |
| | | | | CCUUGCUAU |
| | | | | CCCCAG |
| hsa-miR-6760- | 110 | ACACUGUCC | 352 | CAGUGCAGG |
| 3p | | CCUUCUCCC | | GAGAAGGUG |
| | | CAG | | GAAGUGCAG |
| | | | | AGUGGGCUC |
| | | | | ACCUCUCGC |
| | | | | CCACACUGU |
| | | | | CCCCUUCUC |
| | | | | CCCAG |
| hsa-miR- | 111 | CCCUCUCUG | 353 | CUUCCUGGU |
| 6769b-3p | | UCCCACCCA | | GGGUGGGGA |
| | | UAG | | GGAGAAGUG |
| | | | | CCGUCCUCA |
| | | | | UGAGCCCCU |
| | | | | CUCUGUCCC |
| | | | | ACCCAUAG |
| hsa-miR-6775- | 112 | AGGCCCUGU | 354 | GAACCUCGG |
| 3p | | CCUCUGCCC | | GGCAUGGGG |
| | | CAG | | GAGGGAGGC |
| | | | | UGGACAGGA |
| | | | | GAGGGCUCA |
| | | | | CCCAGGCCC |
| | | | | UGUCCUCUG |
| | | | | CCCCAG |
| hsa-miR-6776- | 113 | CAACCACCA | 355 | CGGGCUCUG |
| 3p | | CUGUCUCUC | | GGUGCAGUG |
| | | CCCAG | | GGGGUUCCC |
| | | | | ACGCCGCGG |
| | | | | CAACCACCA |
| | | | | CUGUCUCUC |
| | | | | CCCAG |
| hsa-miR-6777- | 114 | UCCACUCUC | 356 | UCAAGACGG |
| 3p | | CUGGCCCCC | | GGAGUCAGG |
| | | AG | | CAGUGGUGG |
| | | | | AGAUGGAGA |
| | | | | GCCCUGAGC |
| | | | | CUCCACUCU |
| | | | | CCUGGCCCC |
| | | | | CAG |
| hsa-miR-6782- | 115 | CACCUUUGU | 357 | UGGGGUAGG |
| 3p | | GUCCCCAUC | | GGUGGGGGA |
| | | CUGCA | | AUUCAGGGG |
| | | | | UGUCGAACU |
| | | | | CAUGGCUGC |
| | | | | CACCUUUGU |
| | | | | GUCCCCAUC |
| | | | | CUGCAG |
| hsa-miR-6784- | 116 | UCUCACCCC | 358 | UACAGGCCG |
| 3p | | AACUCUGCC | | GGGCUUUGG |
| | | CCAG | | GUGAGGGAC |
| | | | | CCCCGGAGU |
| | | | | CUGUCACGG |
| | | | | UCUCACCCC |
| | | | | AACUCUGCC |
| | | | | CCAG |
| hsa-miR-6785- | 117 | ACAUCGCCC | 359 | CUCCCUGGG |
| 3p | | CACCUUCCC | | AGGGCGUGG |
| | | CAG | | AUGAUGGUG |
| | | | | GGAGAGGAG |
| | | | | CCCCACUGU |
| | | | | GGAAGUCUG |
| | | | | ACCCCCACA |
| | | | | UCGCCCCAC |
| | | | | CUUCCCCAG |
| hsa-miR-6790- | 118 | CGACCUCGG | 360 | GUGAGUGUG |
| 3p | | CGACCCCUC | | GAUUUGGCG |
| | | ACU | | GGGUUCGGG |
| | | | | GGUUCCGAC |
| | | | | GGCGACCUC |
| | | | | GGCGACCCC |
| | | | | UCACUCACC |
| hsa-miR-6795- | 119 | ACCCCUCGU | 361 | AGGGUUGGG |
| 3p | | UUCUUCCCC | | GGGACAGGA |
| | | CAG | | UGAGAGGCU |
| | | | | GUCUUCAUU |
| | | | | CCCUCUUGA |
| | | | | CCACCCCUC |
| | | | | GUUUCUUCC |
| | | | | CCCAG |
| hsa-miR-6797- | 120 | UGCAUGACC | 362 | CAGCCAGGA |
| 3p | | CUUCCCUCC | | GGGAAGGGG |
| | | CCAC | | CUGAGAACA |
| | | | | GGACCUGUG |
| | | | | CUCACUGGG |
| | | | | GCCUGCAUG |
| | | | | ACCCUUCCC |
| | | | | UCCCCACAG |
| hsa-miR-6799- | 121 | UGCCCUGCA | 363 | GAGGAGGGG |
| 3p | | UGGUGUCCC | | AGGUGUGCA |
| | | CACAG | | GGGCUGGGG |
| | | | | UCACUGACU |
| | | | | CUGCUUCCC |
| | | | | CUGCCCUGC |
| | | | | AUGGUGUCC |
| | | | | CCACAG |
| hsa-miR-6800- | 122 | CACCUCUCC | 364 | ACCUGUAGG |
| 3p | | UGGCAUCGC | | UGACAGUCA |
| | | CCC | | GGGGCGGG |
| | | | | GUGUGGUGG |
| | | | | GGCUGGGGC |
| | | | | UGGCCCCCU |
| | | | | CCUCACACC |
| | | | | UCUCCUGGC |
| | | | | AUCGCCCCC |
| | | | | AG |
| hsa-miR-6801- | 123 | ACCCCUGCC | 365 | UGGCCUGGU |
| 3p | | ACUCACUGG | | CAGAGGCAG |
| | | CC | | CAGGAAAUG |
| | | | | AGAGUUAGC |
| | | | | CAGGAGCUU |
| | | | | UGCAUACUC |
| | | | | ACCCCUGCC |
| | | | | ACUCACUGG |
| | | | | CCCCCAG |
| hsa-miR-6802- | 124 | UUCACCCCU | 366 | GAGGGCUAG |
| 3p | | CUCACCUAA | | GUGGGGGGC |
| | | GCAG | | UUGAAGCCC |
| | | | | CGAGAUGCC |
| | | | | UCACGUCUU |
| | | | | CACCCCUCU |
| | | | | CACCUAAGC |
| | | | | AG |
| hsa-miR-6802- | 125 | CUAGGUGGG | 367 | GAGGGCUAG |
| 5p | | GGGCUUGAA | | GUGGGGGGC |
| | | GC | | UUGAAGCCC |
| | | | | CGAGAUGCC |
| | | | | UCACGUCUU |
| | | | | CACCCCUCU |
| | | | | CACCUAAGC |
| | | | | AG |
| hsa-miR-6803- | 126 | CUGGGGGUG | 368 | CUCCUCUGG |
| 5p | | GGGGGCUGG | | GGGUGGGG |
| | | GCGU | | GGCUGGGCG |
| | | | | UGGUGGACA |
| | | | | GCGAUGCAU |
| | | | | CCCUCGCCU |
| | | | | UCUCACCCU |
| | | | | CAG |
| hsa-miR-6810- | 127 | UCCCCUGCU | 369 | CUGGGAUGG |
| 3p | | CCCUUGUUC | | GGACAGGGA |
| | | CCCAG | | UCAGCAUGG |
| | | | | CACAGAUCC |
| | | | | AAUACCUUC |
| | | | | UGUCCCCUG |
| | | | | CUCCCUUGU |
| | | | | UCCCCAG |
| hsa-miR-6810- | 128 | AUGGGGACA | 370 | CUGGGAUGG |
| 5p | | GGGAUCAGC | | GGACAGGGA |
| | | AUGGC | | UCAGCAUGG |
| | | | | CACAGAUCC |
| | | | | AAUACCUUC |
| | | | | UGUCCCCUG |
| | | | | CUCCCUUGU |
| | | | | UCCCCAG |
| hsa-miR-6812- | 129 | CCGCUCUUC | 371 | UGAGGAUGG |
| 3p | | CCCUGACCC | | GGUGAGAUG |
| | | CAG | | GGGAGGAGC |
| | | | | AGCCAGUCC |
| | | | | UGUCUCACC |
| | | | | GCUCUUCCC |
| | | | | CUGACCCCA |
| | | | | G |
| hsa-miR-6813- | 130 | AACCUUGGC | 372 | GUAGGCAGG |
| 3p | | CCCUCUCCC | | GGCUGGGGU |
| | | CAG | | UUCAGGUUC |
| | | | | UCAGUCAGA |
| | | | | ACCUUGGCC |
| | | | | CCUCUCCCC |
| | | | | AG |
| hsa-miR-6819- | 131 | AAGCCUCUG | 373 | GAGGGUUGG |
| 3p | | UCCCCACCC | | GGUGGAGGG |
| | | CAG | | CCAAGGAGC |
| | | | | UGGGUGGGG |
| | | | | UGCCAAGCC |
| | | | | UCUGUCCCC |
| | | | | ACCCCAG |
| hsa-miR-6819- | 132 | UUGGGGUGG | 374 | GAGGGUUGG |
| 5p | | AGGGCCAAG | | GGUGGAGGG |
| | | GAGC | | CCAAGGAGC |
| | | | | UGGGUGGGG |
| | | | | UGCCAAGCC |
| | | | | UCUGUCCCC |
| | | | | ACCCCAG |
| hsa-miR-6820- | 133 | UGUGACUUC | 375 | CCUUCUGCG |
| 3p | | UCCCCUGCC | | GCAGAGCUG |
| | | ACAG | | GGGUCACCA |
| | | | | GCCCUCAUG |
| | | | | UACUUGUGA |
| | | | | CUUCUCCCC |
| | | | | UGCCACAG |
| hsa-miR-6824- | 134 | UCUCUGGUC | 376 | GAGGUGUAG |
| 3p | | UUGCCACCC | | GGGAGGUUG |
| | | CAG | | GGCCAGGGA |
| | | | | UGCCUUCAC |
| | | | | UGUGUCUCU |
| | | | | CUGGUCUUG |
| | | | | CCACCCCAG |
| hsa-miR-6827- | 135 | ACCGUCUCU | 377 | UCUGGUGGG |
| 3p | | UCUGUUCCC | | AGCCAUGAG |
| | | CAG | | GGUCUGUGC |
| | | | | UGUCUCUGA |
| | | | | GCACCGUCU |
| | | | | CUUCUGUUC |
| | | | | CCCAG |
| hsa-miR-6840- | 136 | ACCCCCGGG | 378 | UGACCACCC |
| 5p | | CAAAGACCU | | CCGGGCAAA |
| | | GCAGAU | | GACCUGCAG |
| | | | | AUCCCCUGU |
| | | | | UAGAGACGG |
| | | | | GCCCAGGAC |
| | | | | UUUGUGCGG |
| | | | | GGUGCCCA |
| hsa-miR-6841- | 137 | ACCUUGCAU | 379 | GUGUUUAGG |
| 3p | | CUGCAUCCC | | GUACUCAGA |
| | | CAG | | GCAAGUUGU |
| | | | | GAAACACAG |
| | | | | GUGUUUUUU |
| | | | | AACCUCACC |
| | | | | UUGCAUCUG |
| | | | | CAUCCCCAG |
| hsa-miR-6846- | 138 | UGACCCCUU | 380 | CAGGCUGGG |
| 3p | | CUGUCUCCC | | GGCUGGAUG |
| | | UAG | | GGGUAGAGU |
| | | | | AGGAGAGCC |
| | | | | CACUGACCC |
| | | | | CUUCUGUCU |
| | | | | CCCUAG |
| hsa-miR-6846- | 139 | UGGGGGCUG | 381 | CAGGCUGGG |
| 5p | | GAUGGGGUA | | GGCUGGAUG |
| | | GAGU | | GGGUAGAGU |
| | | | | AGGAGAGCC |
| | | | | CACUGACCC |
| | | | | CUUCUGUCU |
| | | | | CCCUAG |
| hsa-miR-6848- | 140 | GUGGUCUCU | 382 | GUCCCUGGG |
| 3p | | UGGCCCCCA | | GGCUGGGAU |
| | | G | | GGGCCAUGG |
| | | | | UGUGCUCUG |
| | | | | AUCCCCCUG |
| | | | | UGGUCUCUU |
| | | | | GGCCCCCAG |
| | | | | GAACUCC |
| hsa-miR-6855- | 141 | AGACUGACC | 383 | GCUGCUUGG |
| 3p | | UUCAACCCC | | GGUUUGGGG |
| | | ACAG | | UGCAGACAU |
| | | | | UGCCAGAGG |
| | | | | AUGGGCAGC |
| | | | | AGACUGACC |
| | | | | UUCAACCCC |
| | | | | ACAG |
| hsa-miR-6857- | 142 | UGACUGAGC | 384 | GCUUGUUGG |
| 3p | | UUCUCCCCA | | GGAUUGGGU |
| | | CAG | | CAGGCCAGU |
| | | | | GUUCAAGGG |
| | | | | CCCCUCCUC |
| | | | | UAGUACUCC |
| | | | | CUGUUUGUG |
| | | | | UUCUGCCAC |
| | | | | UGACUGAGC |
| | | | | UUCUCCCCA |
| | | | | CAG |
| hsa-miR-6861- | 143 | UGGACCUCU | 385 | GAGGCACUG |
| 3p | | CCUCCCCAG | | GGUAGGUGG |
| | | | | GGCUCCAGG |
| | | | | GCUCCUGAC |
| | | | | ACCUGGACC |
| | | | | UCUCCUCCC |
| | | | | CAGGCCCAC |
| | | | | A |
| hsa-miR-6862- | 144 | CGGGCAUGC | 386 | CGAAGCGGG |
| 5p | | UGGGAGAGA | | CAUGCUGGG |
| | | CUUU | | AGAGACUUU |
| | | | | GUGAUUUGU |
| | | | | CUCCAAAGC |
| | | | | CUCACCCAG |
| | | | | CUCUCUGGC |
| | | | | CCUCUAG |
| hsa-miR-6870- | 145 | GCUCAUCCC | 387 | CAAGGUGGG |
| 3p | | CAUCUCCUU | | GGAGAUGGG |
| | | UCAG | | GGUUGAACU |
| | | | | UCAUUUCUC |
| | | | | AUGCUCAUC |
| | | | | CCCAUCUCC |
| | | | | UUUCAG |
| hsa-miR-6872- | 146 | CCCAUGCCU | 388 | GUGGGUCUC |
| 3p | | CCUGCCGCG | | GCAUCAGGA |
| | | GUC | | GGCAAGGCC |
| | | | | AGGACCCGC |
| | | | | UGACCCAUG |
| | | | | CCUCCUGCC |
| | | | | GCGGUCAG |
| hsa-miR-6878- | 147 | AGGGAGAAA | 389 | AUGAGAGGG |
| 5p | | GCUAGAAGC | | AGAAAGCUA |
| | | UGAAG | | GAAGCUGAA |
| | | | | GAUUCUGAA |
| | | | | AAUCACUAA |
| | | | | CUGGCCUCU |
| | | | | UCUUUCUCC |
| | | | | UAG |
| hsa-miR-6880- | 148 | CCGCCUUCU | 390 | GAGGGUGGU |
| 3p | | CUCCUCCCC | | GGAGGAAGA |
| | | CAG | | GGGCAGCUC |
| | | | | CCAUGACUG |
| | | | | CCUGACCGC |
| | | | | CUUCUCUCC |
| | | | | UCCCCCAG |
| hsa-miR-6884- | 149 | CCCAUCACC | 391 | CCCGCAGAG |
| 3p | | UUUCCGUCU | | GCUGAGAAG |
| | | CCCCU | | GUGAUGUUG |
| | | | | GCUCAAGAA |
| | | | | AGGGAGAUA |
| | | | | GAUGGUAGC |
| | | | | CCAUCACCU |
| | | | | UUCCGUCUC |
| | | | | CCCUAG |
| hsa-miR-6885- | 150 | CUUUGCUUC | 392 | CCUGGAGGG |
| 3p | | CUGCUCCCC | | GGGCACUGC |
| | | UAG | | GCAAGCAAA |
| | | | | GCCAGGGAC |
| | | | | CCUGAGAGG |
| | | | | CUUUGCUUC |
| | | | | CUGCUCCCC |
| | | | | UAG |
| hsa-miR-6887- | 151 | UCCCCUCCA | 393 | GAGAAUGGG |
| 3p | | CUUUCCUCC | | GGGACAGAU |
| | | UAG | | GGAGAGGAC |
| | | | | ACAGGCUGG |
| | | | | CACUGAGGU |
| | | | | CCCCUCCAC |
| | | | | UUUCCUCCU |
| | | | | AG |
| hsa-miR-6889- | 152 | UCUGUGCCC | 394 | CUGUGUCGG |
| 3p | | CUACUUCCC | | GGAGUCUGG |
| | | AG | | GGUCCGGAA |
| | | | | UUCUCCAGA |
| | | | | GCCUCUGUG |
| | | | | CCCCUACUU |
| | | | | CCCAG |
| hsa-miR-6892- | 153 | UCCCUCUCC | 395 | GUAAGGGAC |
| 3p | | CACCCCUUG | | CGGAGAGUA |
| | | CAG | | GGAAAAGCA |
| | | | | GGGCUCAGG |
| | | | | GCCAGAGAG |
| | | | | ACUGGGCAU |
| | | | | AGAACUAAG |
| | | | | GAGGAUGGU |
| | | | | GUCCUCCUG |
| | | | | ACUGCAUCU |
| | | | | CUCUUCCCU |
| | | | | CUCCCACCC |
| | | | | CUUGCAG |
| hsa-miR-7114- | 154 | UGACCCACC | 396 | UCCGCUCUG |
| 3p | | CCUCUCCAC | | UGGAGUGGG |
| | | CAG | | GUGCCUGUC |
| | | | | CCCUGCCAC |
| | | | | UGGGUGACC |
| | | | | CACCCCUCU |
| | | | | CCACCAG |
| hsa-miR-7150 | 155 | CUGGCAGGG | 397 | CACGGUGUC |
| | | GGAGAGGUA | | CCCUGGUGG |
| | | | | AACCUGGCA |
| | | | | GGGGGAGAG |
| | | | | GUAAGGUCU |
| | | | | UUCAGCCUC |
| | | | | UCCAAAGCC |
| | | | | CAUGGUCAG |
| | | | | GUACUCAGG |
| | | | | UGGGGGAGC |
| | | | | CCUG |
| hsa-miR-767- | 156 | UCUGCUCAU | 398 | GCUUUUAUA |
| 3p | | ACCCCAUGG | | UUGUAGGUU |
| | | UUUCU | | UUUGCUCAU |
| | | | | GCACCAUGG |
| | | | | UUGUCUGAG |
| | | | | CAUGCAGCA |
| | | | | UGCUUGUCU |
| | | | | GCUCAUACC |
| | | | | CCAUGGUUU |
| | | | | CUGAGCAGG |
| | | | | AACCUUCAU |
| | | | | UGUCUACUG |
| | | | | C |
| hsa-miR-8087 | 157 | GAAGACUUC | 399 | UCUAAGAAG |
| | | UUGGAUUAC | | UGAAGACUU |
| | | AGGGG | | CUUGGAUUA |
| | | | | CAGGGGCCC |
| | | | | UACUUUAAG |
| | | | | GGCCCUUUC |
| | | | | AGUUGGAAG |
| | | | | UUUUCCUUU |
| | | | | CUGCCU |
| hsa-miR-874- | 158 | CGGCCCCAC | 400 | UUAGCCCUG |
| 5p | | GCACCAGGG | | CGGCCCCAC |
| | | UAAGA | | GCACCAGGG |
| | | | | UAAGAGAGA |
| | | | | CUCUCGCUU |
| | | | | CCUGCCCUG |
| | | | | GCCCGAGGG |
| | | | | ACCGACUGG |
| | | | | CUGGGC |
| hsa-miR-920 | 159 | GGGGAGCUG | 401 | GUAGUUGUU |
| | | UGGAAGCAG | | CUACAGAAG |
| | | UA | | ACCUGGAUG |
| | | | | UGUAGGAGC |
| | | | | UAAGACACA |
| | | | | CUCCAGGGG |
| | | | | AGCUGUGGA |
| | | | | AGCAGUAAC |
| | | | | ACG |
| hsa-miR-98-3p | 160 | CUAUACAAC | 402 | AGGAUUCUG |
| | | UUACUACUU | | CUCAUGCCA |
| | | UCCC | | GGGUGAGGU |
| | | | | AGUAAGUUG |
| | | | | UAUUGUUGU |
| | | | | GGGGUAGGG |
| | | | | AUAUUAGGC |
| | | | | CCCAAUUAG |
| | | | | AAGAUAACU |
| | | | | AUACAACUU |
| | | | | ACUACUUUC |
| | | | | CCUGGUGUG |
| | | | | UGGCAUAUU |
| | | | | CA |

TABLE 3
82 down-regulated miRNAs associated with SLE

| | SEQ | | SEQ | |
|---|---|---|---|---|
| | ID | | ID | |
| miRNA | NO: | Mature Sequence | NO: | Precursor Sequence |
| hsa-miR-10394- | 161 | UGGGCGCGCCG | 403 | UCUGCAGGUCCUGGUGAAC |
| 3p | | GGACUGUGAGA | | GCCAUCAUCAACAGUGGUCC |
| | | C | | CCGGGAGGACUCCACACGC |
| | | | | AUUGGGCGCGCCGGGACUG |
| | | | | UGAGAC |
| hsa-miR-10396a- | 162 | GGCGGGGCUCG | 404 | GGCGGGGCUCGGAGCCGGG |
| 5p | | GAGCCGGG | | CUUCGGCCGGGCCCCGGGC |
| | | | | CCUCGACCGGG |
| hsa-miR-10396b- | 163 | CGGCGGGGCUC | 405 | CGGCGGGGCUCGGAGCCGG |
| 5p | | GGAGCCGGG | | GCUUCGGCCGGGCCCCGGG |
| | | | | CCCUCGACCGGAC |
| hsa-miR-10400- | 164 | CGGCGGCGGCG | 406 | CGGCGGCGGCGGCUCUGGG |
| 5p | | GCUCUGGGCG | | CGAGGCGGCGGGGCCUGGG |
| | | | | CUCCCGGACGAGGGGGG |
| hsa-miR-12118 | 165 | CAAGGAGGAGC | 407 | GGGUCAAGGAGGAGCGGGG |
| | | GGGGAUUAG | | AUUAGUUCUAGGGGCUGUA |
| | | | | GGAGGGUGACAGUCCUGGA |
| | | | | CUGAAGGUCACCUGCUUGG |
| | | | | CUCUGAUGAUUU |
| hsa-miR-12120 | 166 | UAAGGAACGCG | 408 | CUGGCUGGGCGGUAAGGAA |
| | | GGGCCUUGGUA | | CGCGGGGCCUUGGUAGAGC |
| | | GAGC | | AAAGUGCGGACCAAAGACUU |
| | | | | UGCGUCUGGUUGCUUUUAC |
| | | | | CUUGCCUAGUAGG |
| hsa-miR-12121 | 167 | CUGCCACGAGC | 409 | UGGGCUCGGCCCGGGCUGC |
| | | GUGCGGGCCU | | CACGAGCGUGCGGGCCUCG |
| | | | | CCGGGCAUGUCCUAGGCGG |
| | | | | CGGCCCCGCCCAGCGCUCG |
| | | | | GCCGGGGGGGGGGGGGGGC |
| | | | | GCG |
| hsa-miR-1228- | 168 | GUGGGGGGGGG | 410 | GUGGGGGGGGGCAGGUGUG |
| 5p | | CAGGUGUGUG | | UGGUGGGUGGUGGCCUGCG |
| | | | | GUGAGCAGGGCCCUCACAC |
| | | | | CUGCCUCGCCCCCCAG |
| hsa-miR-1231 | 169 | GUGUCUGGGCG | 411 | GUCAGUGUCUGGGGGGACA |
| | | GACAGCUGC | | GCUGCAGGAAAGGGAAGAC |
| | | | | CAAGGCUUGCUGUCUGUCC |
| | | | | AGUCUGCCACCCUACCCUGU |
| | | | | CUGUUCUUGCCACAG |
| hsa-miR-1237- | 170 | CGGGGGCGGGG | 412 | GUGGGAGGGCCCAGGCGCG |
| 5p | | CCGAAGCGCG | | GGCAGGGGUGGGGGUGGCA |
| | | | | GAGCGCUGUCCCGGGGGCG |
| | | | | GGGCCGAAGCGCGGCGACC |
| | | | | GUAACUCCUUCUGCUCCGU |
| | | | | CCCCCAG |
| hsa-miR-1268a | 171 | CGGGCGUGGUG | 413 | UAGCCGGGCGUGGUGGUGG |
| | | GUGGGGG | | GGGCCUGUGGUCCCAGCUA |
| | | | | CUUUGGAGGCUGAG |
| hsa-miR-1268b | 172 | CGGGCGUGGUG | 414 | ACCCGGGCGUGGUGGUGGG |
| | | GUGGGGGUG | | GGUGGGUGCCUGUAAUUCC |
| | | | | AGCUAGUUGGGA |
| hsa-miR-128-1- | 173 | CGGGGCCGUAG | 415 | UGAGCUGUUGGAUUCGGGG |
| 5p | | CACUGUCUGAG | | CCGUAGCACUGUCUGAGAG |
| | | A | | GUUUACAUUUCUCACAGUGA |
| | | | | ACCGGUCUCUUUUUCAGCU |
| | | | | GCUUC |
| hsa-miR-1469 | 174 | CUCGGGGGGGG | 416 | CUCGGCGCGGGGCGCGGGC |
| | | GCGCGGGCUCC | | UCCGGGUUGGGGCGAGCCA |
| | | | | ACGCCGGGG |
| hsa-miR-1908-5p | 175 | CGGCGGGGACG | 417 | CGGGAAUGCCGCGGCGGGG |
| | | GCGAUUGGUC | | ACGGCGAUUGGUCCGUAUG |
| | | | | UGUGGUGCCACCGGCCGCC |
| | | | | GGCUCCGCCCCGGCCCCCG |
| | | | | CCCC |
| hsa-miR-1909-3p | 176 | CGCAGGGGCCG | 418 | CAUCCAGGACAAUGGUGAGU |
| | | GGUGCUCACCG | | GCCGGUGCCUGCCCUGGGG |
| | | | | CCGUCCCUGCGCAGGGGCC |
| | | | | GGGUGCUCACCGCAUCUGC |
| | | | | CCC |
| hsa-miR-1915-3p | 177 | CCCCAGGGCGA | 419 | UGAGAGGCCGCACCUUGCC |
| | | CGCGGCGGG | | UUGCUGCCCGGGCCGUGCA |
| | | | | CCCGUGGGCCCCAGGGCGA |
| | | | | CGCGGGGGGGGCGGCCCUA |
| | | | | GCGA |
| hsa-miR-3178 | 178 | GGGGCGCGGCC | 420 | GAGGCUGGGGGGGGGGGG |
| | | GGAUCG | | CCGGAUCGGUCGAGAGCGU |
| | | | | CCUGGCUGAUGACGGUCUC |
| | | | | CCGUGCCCACGCCCCAAACG |
| | | | | CAGUCUC |
| hsa-miR-3180-3p | 179 | UGGGGCGGAGC | 421 | CAGUGCGACGGGCGGAGCU |
| | | UUCCGGAGGCC | | UCCAGACGCUCCGCCCCAC |
| | | | | GUCGCAUGCGCCCCGGGAA |
| | | | | AGCGUGGGGCGGAGCUUCC |
| | | | | GGAGGCCCCGCCCUGCUG |
| hsa-miR-3195 | 180 | CGCGCCGGGCC | 422 | CCGCAGCCGCCGCGCCGGG |
| | | CGGGUU | | CCCGGGUUGGCCGCUGACC |
| | | | | CCCGCGGGGCCCCCGGCGG |
| | | | | CCGGGGGGGGGGGGGGGG |
| | | | | CUGCCCCGG |
| hsa-miR-3196 | 181 | CGGGGCGGCAG | 423 | GGGUGGGGGGGGGGGGCA |
| | | GGGCCUC | | GGGGCCUCCCCCAGUGCCA |
| | | | | GGCCCCAUUCUGCUUCUCU |
| | | | | CCCAGCU |
| hsa-miR-3620-5p | 182 | GUGGGCUGGGC | 424 | GUGAGGUGGGGGCCAGCAG |
| | | UGGGCUGGGCC | | GGAGUGGGCUGGGCUGGGC |
| | | | | UGGGCCAAGGUACAAGGCC |
| | | | | UCACCCUGCAUCCCGCACCC |
| | | | | AG |
| hsa-miR-3621 | 183 | CGCGGGUCGGG | 425 | GUGAGCUGCUGGGGACGCG |
| | | GUCUGCAGG | | GGUCGGGGUCUGCAGGGCG |
| | | | | GUGCGGCAGCCGCCACCUG |
| | | | | ACGCCGCGCCUUUGUCUGU |
| | | | | GUCCCACAG |
| hsa-miR-3663-3p | 184 | UGAGCACCACA | 426 | CCCGGGACCUUGGUCCAGG |
| | | CAGGCCGGGCG | | CGCUGGUCUGCGUGGUGCU |
| | | C | | CGGGUGGAUAAGUCUGAUC |
| | | | | UGAGCACCACACAGGCCGG |
| | | | | GCGCCGGGACCAAGGGGGC |
| | | | | UC |
| hsa-miR-3665 | 185 | AGCAGGUGCGG | 427 | GCGGGGGGGGGGGGCGGCA |
| | | GGCGGCG | | GCAGCAGCAGGUGCGGGGC |
| | | | | GGCGGCCGCGCUGGCCGCU |
| | | | | CGACUCCGCAGCUGCUCGU |
| | | | | UCUGCUUCUCCAGCUUGCG |
| | | | | CACCAGCUCC |
| hsa-miR-3940-5p | 186 | GUGGGUUGGGG | 428 | GCUUAUCGAGGAAAAGAUCG |
| | | CGGGCUCUG | | AGGUGGGUUGGGGCGGGCU |
| | | | | CUGGGGAUUUGGUCUCACA |
| | | | | GCCCGGAUCCCAGCCCACU |
| | | | | UACCUUGGUUACUCUCCUUC |
| | | | | CUUCU |
| hsa-miR-4443 | 187 | UUGGAGGCGUG | 429 | GGUGGGGGUUGGAGGCGUG |
| | | GGUUUU | | GGUUUUAGAACCUAUCCCUU |
| | | | | UCUAGCCCUGAGCA |
| hsa-miR-4446-3p | 188 | CAGGGCUGGCA | 430 | CUGGUCCAUUUCCCUGCCA |
| | | GUGACAUGGGU | | UUCCCUUGGCUUCAAUUUAC |
| | | | | UCCCAGGGCUGGCAGUGAC |
| | | | | AUGGGUCAA |
| hsa-miR-4492 | 189 | GGGGCUGGGCG | 431 | CUGCAGCGUGCUUCUCCAG |
| | | CGCGCC | | GCCCCGCGCGCGGACAGAC |
| | | | | ACACGGACAAGUCCCGCCAG |
| | | | | GGGCUGGGCGCGCGCCAGC |
| | | | | CGG |
| hsa-miR-4497 | 190 | CUCCGGGACGG | 432 | ACCUCCGGGACGGCUGGGC |
| | | CUGGGC | | GCCGGCGGCCGGGAGAUCC |
| | | | | GCGCUUCCUGAAUCCCGGC |
| | | | | CGGCCCGCCCGGCGCCCGU |
| | | | | CCGCCCGCGGGUC |
| hsa-miR-4505 | 191 | AGGCUGGGCUG | 433 | GGAGGCUGGGCUGGGACGG |
| | | GGACGGA | | ACACCCGGCCUCCACUUUCU |
| | | | | GUGGCAGGUACCUCCUCCA |
| | | | | UGUCGGCCCGCCUUG |
| hsa-miR-4508 | 192 | GCGGGGCUGGG | 434 | AGGACCCAGCGGGGCUGGG |
| | | CGCGCG | | CGCGCGGAGCAGCGCUGGG |
| | | | | UGCAGCGCCUGCGCCGGCA |
| | | | | GCUGCAAGGGCCG |
| hsa-miR-4516 | 193 | GGGAGAAGGGU | 435 | AGGGAGAAGGGUCGGGGCA |
| | | CGGGGC | | GGGAGGGCAGGGCAGGCUC |
| | | | | UGGGGUGGGGGGUCUGUGA |
| | | | | GUCAGCCACGGCUCUGCCC |
| | | | | ACGUCUCCCC |
| hsa-miR-4530 | 194 | CCCAGCAGGAC | 436 | CGACCGCACCCGCCCGAAG |
| | | GGGAGCG | | CUGGGUCAAGGAGCCCAGC |
| | | | | AGGACGGGAGCGCGGCGC |
| hsa-miR-4634 | 195 | CGGCGCGACCG | 437 | GGACAAGGGCGGCGCGACC |
| | | GCCCGGGG | | GGCCCGGGGCUCUUGGGCG |
| | | | | GCCGCGUUUCCCCUCC |
| hsa-miR-4655-5p | 196 | CACCGGGGAUG | 438 | CCAAGGGCACACCGGGGAU |
| | | GCAGAGGGUCG | | GGCAGAGGGUCGUGGGAAA |
| | | | | GUGUUGACCCUCGUCAGGU |
| | | | | CCCCGGGGAGCCCCUGG |
| hsa-miR-4674 | 197 | CUGGGCUCGGG | 439 | CCCAGGCGCCCGCUCCCGA |
| | | ACGCGCGGCU | | CCCACGCCGCGCCGCCGGG |
| | | | | UCCCUCCUCCCCGGAGAGG |
| | | | | CUGGGCUCGGGACGCGCGG |
| | | | | CUCAGCUCGGG |
| hsa-miR-4688 | 198 | UAGGGGCAGCA | 440 | GUCUACUCCCAGGGUGCCA |
| | | GAGGACCUGGG | | AGCUGUUUCGUGUUCCCUC |
| | | | | CCUAGGGGAUCCCAGGUAG |
| | | | | GGGCAGCAGAGGACCUGGG |
| | | | | CCUGGAC |
| hsa-miR-4707-5p | 199 | GCCCCGGCGCG | 441 | GGUUCCGGAGCCCCGGCGC |
| | | GGCGGGUUCUG | | GGGCGGGUUCUGGGGUGUA |
| | | G | | GACGCUGCUGGCCAGCCCG |
| | | | | CCCCAGCCGAGGUUCUCGG |
| | | | | CACC |
| hsa-miR-4722-5p | 200 | GGCAGGAGGGC | 442 | GGCAGGAGGGCUGUGCCAG |
| | | UGUGCCAGGUU | | GUUGGCUGGGCCAGGCCUG |
| | | G | | ACCUGCCAGCACCUCCCUGC |
| | | | | AG |
| hsa-miR-4730 | 201 | CUGGCGGAGCC | 443 | CGCAGGCCUCUGGCGGAGC |
| | | CAUUCCAUGCC | | CCAUUCCAUGCCAGAUGCUG |
| | | A | | AGCGAUGGCUGGUGUGUGC |
| | | | | UGCUCCACAGGCCUGGUG |
| hsa-miR-4734 | 202 | GCUGCGGGCUG | 444 | CUCGGGCCCGACCGCGCCG |
| | | CGGUCAGGGCG | | GCCCGCACCUCCCGGCCCG |
| | | | | GAGCUGCGGGCUGCGGUCA |
| | | | | GGGCGAUCCCGGG |
| hsa-miR-4750-5p | 203 | CUCGGGGGGAG | 445 | CGCUCGGGGGGAGGUGGUU |
| | | GUGGUUGAGUG | | GAGUGCCGACUGGCGCCUG |
| | | | | ACCCACCCCCUCCCGCAG |
| hsa-miR-4763-3p | 204 | AGGCAGGGGCU | 446 | CCUGUCCCUCCUGCCCUGC |
| | | GGUGCUGGGCG | | GCCUGCCCAGCCCUCCUGC |
| | | GG | | UCUGGUGACUGAGGACCGC |
| | | | | CAGGCAGGGGCUGGUGCUG |
| | | | | GGCGGGGGGGGGGGGG |
| hsa-miR-4787-5p | 205 | GCGGGGGUGGC | 447 | CGGUCCAGACGUGGCGGGG |
| | | GGCGGCAUCCC | | GUGGCGGCGGCAUCCCGGA |
| | | | | CGGCCUGUGAGGGAUGCGC |
| | | | | CGCCCACUGCCCCGCGCCG |
| | | | | CCUGACCG |
| hsa-miR-486-3p | 206 | CGGGGCAGCUC | 448 | GCAUCCUGUACUGAGCUGC |
| | | AGUACAGGAU | | CCCGAGGCCCUUCAUGCUG |
| | | | | CCCAGCUCGGGGCAGCUCA |
| | | | | GUACAGGAUAC |
| hsa-miR-5090 | 207 | CCGGGGCAGAU | 449 | UCUGAGGUACCCGGGGCAG |
| | | UGGUGUAGGGU | | AUUGGUGUAGGGUGCAAAG |
| | | G | | CCUGCCCGCCCCCUAAGCC |
| | | | | UUCUGCCCCCAACUCCAGCC |
| | | | | UGUCAGGA |
| hsa-miR-575 | 208 | GAGCCAGUUGG | 450 | AAUUCAGCCCUGCCACUGGC |
| | | ACAGGAGC | | UUAUGUCAUGACCUUGGGC |
| | | | | UACUCAGGCUGUCUGCACAA |
| | | | | UGAGCCAGUUGGACAGGAG |
| | | | | CAGUGCCACUCAACUC |
| hsa-miR-5787 | 209 | GGGCUGGGGCG | 451 | GGGGGCUGGGGCGCGGGGA |
| | | CGGGGAGGU | | GGUGCUAGGUCGGCCUCGG |
| | | | | CUCCCGCGCCGCACCCC |
| hsa-miR-6075 | 210 | ACGGCCCAGGC | 452 | GACACCACAUGCUCCUCCAG |
| | | GGCAUUGGUG | | GCCUGCCUGCCCUCCAGGU |
| | | | | CAUGUUCCAGUGUCCCACAG |
| | | | | AUGCAGCACCACGGCCCAG |
| | | | | GCGGCAUUGGUGUCACC |
| hsa-miR-6125 | 211 | GCGGAAGGCGG | 453 | GCUCUGGGGCGUGCCGCCG |
| | | AGCGGCGGA | | CCGUCGCUGCCACCUCCCC |
| | | | | UACCGCUAGUGGAAGAAGAU |
| | | | | GGCGGAAGGCGGAGCGGCG |
| | | | | GAUCUGGACACCCAGCGGU |
| hsa-miR-6126 | 212 | GUGAAGGCCCG | 454 | AGCCUGUGGGAAAGAGAAGA |
| | | GCGGAGA | | GCAGGGCAGGGUGAAGGCC |
| | | | | CGGCGGAGACACUCUGCCC |
| | | | | ACCCCACACCCUGCCUAUGG |
| | | | | GCCACACAGCU |
| hsa-miR-638 | 213 | AGGGAUCGCGG | 455 | GUGAGCGGGCGCGGCAGGG |
| | | GCGGGUGGCGG | | AUCGGGGGGGGGUGGCGGC |
| | | CCU | | CUAGGGGGGGGAGGGCGGA |
| | | | | CCGGGAAUGGCGCGCCGUG |
| | | | | CGCCGCCGGCGUAACUGCG |
| | | | | GCGCU |
| hsa-miR-663a | 214 | AGGCGGGGCGC | 456 | CCUUCCGGCGUCCCAGGCG |
| | | CGCGGGACCGC | | GGGCGCCGCGGGACCGCCC |
| | | | | UCGUGUCUGUGGCGGUGGG |
| | | | | AUCCCGCGGCCGUGUUUUC |
| | | | | CUGGUGGCCCGGCCAUG |
| hsa-miR-6721-5p | 215 | UGGGCAGGGGC | 457 | CCCUCAUCUCUGGGCAGGG |
| | | UUAUUGUAGGA | | GCUUAUUGUAGGAGUCUCU |
| | | G | | GAAGAGAGCUGUGGACUGA |
| | | | | CCUGCUUUAACCCUUCCCCA |
| | | | | GGUUCCCAUU |
| hsa-miR-6724-5p | 216 | CUGGGCCCGCG | 458 | CGCUGCGCUUCUGGGCCCG |
| | | GCGGGCGUGGG | | CGGCGGGCGUGGGGCUGCC |
| | | G | | CGGGCCGGUCGACCAGCGC |
| | | | | GCCGUAGCUCCCGAGGCCC |
| | | | | GAGCCGCGACCCGCGG |
| hsa-miR-6729-5p | 217 | UGGGCGAGGGC | 459 | GAGGGUGGGCGAGGGCGGC |
| | | GGCUGAGCGGC | | UGAGCGGCUCCAUCCCCCG |
| | | | | GCCUGCUCAUCCCCCUCGC |
| | | | | CCUCUCAG |
| hsa-miR-6743-5p | 218 | AAGGGGCAGGG | 460 | GGGUAAAGGGGCAGGGACG |
| | | ACGGGUGGCCC | | GGUGGCCCCAGGAAGAAGG |
| | | | | GCCUGGUGGAGCCGCUCUU |
| | | | | CUCCCUGCCCACAG |
| hsa-miR-6762-5p | 219 | CGGGGCCAUGG | 461 | AGAGCCGGGGCCAUGGAGC |
| | | AGCAGCCUGUG | | AGCCUGUGUAGACGGGGAC |
| | | U | | CUGCCCUGCAUGGGCACCC |
| | | | | CCUCACUGGCUGCUUCCCU |
| | | | | UGGUCUCCAG |
| hsa-miR-6768-5p | 220 | CACACAGGAAAA | 462 | CCAGGCACACAGGAAAAGCG |
| | | GCGGGGCCCUG | | GGGCCCUGGGUUCGGCUGC |
| | | | | UACCCCAAAGGCCACAUUCU |
| | | | | CCUGUGCACACAG |
| hsa-miR-6781-5p | 221 | CGGGCCGGAGG | 463 | AACCCCGGGCCGGAGGUCA |
| | | UCAAGGGCGU | | AGGGCGUCGCUUCUCCCUA |
| | | | | AUGUUGCCUCUUUUCCACG |
| | | | | GCCUCAG |
| hsa-miR-6784-5p | 222 | GCCGGGGCUUU | 464 | UACAGGCCGGGGCUUUGGG |
| | | GGGUGAGGG | | UGAGGGACCCCCGGAGUCU |
| | | | | GUCACGGUCUCACCCCAACU |
| | | | | CUGCCCCAG |
| hsa-miR-6787-5p | 223 | UGGCGGGGGUA | 465 | UCGGCUGGGGGGGGUAGAG |
| | | GAGCUGGCUGC | | CUGGCUGCAGGCCCGGCCC |
| | | | | CUCUCAGCUGCUGCCCUCU |
| | | | | CCAG |
| hsa-miR-6789-5p | 224 | GUAGGGGCGUC | 466 | CGAGGUAGGGGCGUCCCGG |
| | | CCGGGCGCGCG | | GCGCGCGGGGGGGUCCCAG |
| | | GG | | GCUGGGCCCCUCGGAGGCC |
| | | | | GGGUGCUCACUGCCCCGUC |
| | | | | CCGGCGCCCGUGUCUCCUC |
| | | | | CAG |
| hsa-miR-6791-5p | 225 | CCCCUGGGGCU | 467 | CCAGACCCCUGGGGCUGGG |
| | | GGGCAGGCGGA | | CAGGCGGAAAGAGGUCUGA |
| | | | | ACUGCCUCUGCCUCCUUGG |
| | | | | UCUCCGGCAG |
| hsa-miR-6798-5p | 226 | CCAGGGGGAUG | 468 | GGCAGCCAGGGGGAUGGGC |
| | | GGCGAGCUUGG | | GAGCUUGGGCCCAUUCCUU |
| | | G | | UCCUUACCCUACCCCCCAUC |
| | | | | CCCCUGUAG |
| hsa-miR-6800-5p | 227 | GUAGGUGACAG | 469 | ACCUGUAGGUGACAGUCAG |
| | | UCAGGGGCGG | | GGGGGGGGUGUGGUGGGG |
| | | | | CUGGGGCUGGCCCCCUCCU |
| | | | | CACACCUCUCCUGGCAUCGC |
| | | | | CCCCAG |
| hsa-miR-6805-5p | 228 | UAGGGGGGGGC | 470 | UGGCCUAGGGGGGGGCUUG |
| | | UUGUGGAGUGU | | UGGAGUGUAUGGGCUGAGC |
| | | | | CUUGCUCUGCUCCCCCGCC |
| | | | | CCCAG |
| hsa-miR-6816-5p | 229 | UGGGGGGGGGC | 471 | CCGAGUGGGGCGGGGCAGG |
| | | AGGUCCCUGC | | UCCCUGCAGGGACUGUGAC |
| | | | | ACUGAAGGACCUGCACCUUC |
| | | | | GCCCACAG |
| hsa-miR-6821-5p | 230 | GUGCGUGGUGG | 472 | GUGCGUGGUGGCUCGAGGC |
| | | CUCGAGGCGGG | | GGGGGUGGGGGCCUCGCCC |
| | | G | | UGCUUGGGCCCUCCCUGAC |
| | | | | CUCUCCGCUCCGCACAG |
| hsa-miR-6845-5p | 231 | CGGGGCCAGAG | 473 | AACUGCGGGGCCAGAGCAG |
| | | CAGAGAGC | | AGAGCCCUUGCACACCACCA |
| | | | | GCCUCUCCUCCCUGUGCCC |
| | | | | CAG |
| hsa-miR-6850-5p | 232 | GUGCGGAACGC | 474 | GUGCGGAACGCUGGCCGGG |
| | | UGGCCGGGGCG | | GCGGGAGGGGAAGGGACGC |
| | | | | CCGGCCGGAACGCCGCACU |
| | | | | CACG |
| hsa-miR-6869-5p | 233 | GUGAGUAGUGG | 475 | GUGAGUAGUGGCGCGCGGC |
| | | CGCGCGGCGGC | | GGCUCGGAGUACCUCUGCC |
| | | | | GCCGCGCGCAUCGGCUCAG |
| | | | | CAUGC |
| hsa-miR-7108-5p | 234 | GUGUGGCCGGC | 476 | GUGUGGCCGGCAGGGGGGU |
| | | AGGCGGGUGG | | GGGGGGGGGGGGCCGGUG |
| | | | | GGAACCCCGCCCCGCCCCG |
| | | | | CGCCCGCACUCACCCGCCC |
| | | | | GUCUCCCCACAG |
| hsa-miR-744-5p | 235 | UGCGGGGCUAG | 477 | UUGGGCAAGGUGCGGGGCU |
| | | GGCUAACAGCA | | AGGGCUAACAGCAGUCUUAC |
| | | | | UGAAGGUUUCCUGGAAACCA |
| | | | | CGCACAUGCUGUUGCCACUA |
| | | | | ACCUCAACCUUACUCGGUC |
| hsa-miR-762 | 236 | GGGGCUGGGGC | 478 | GGCCCGGCUCCGGGUCUCG |
| | | CGGGGCCGAGC | | GCCCGUACAGUCCGGCCGG |
| | | | | CCAUGCUGGCGGGGCUGGG |
| | | | | GCCGGGGCCGAGCCCGCGG |
| | | | | CGGGGCC |
| hsa-miR-7704 | 237 | CGGGGUCGGCG | 479 | CGGGGUCGGCGGCGACGUG |
| | | GCGACGUG | | CUCAGCUUGGCACCCAAGUU |
| | | | | CUGCCGCUCCGACGCCCGG |
| | | | | C |
| hsa-miR-8063 | 238 | UCAAAAUCAGGA | 480 | UAGAGGCAGUUUCAACAGAU |
| | | GUCGGGGCUU | | GUGUAGACUUUUGAUAUGA |
| | | | | GAAAUUGGUUUCAAAAUCAG |
| | | | | GAGUCGGGGCUUUACUGCU |
| | | | | UUU |
| hsa-miR-8069 | 239 | GGAUGGUUGGG | 481 | CGCCUGAGCGUGCAGCAGG |
| | | GGCGGUCGGCG | | ACAUCUUCCUGACCUGGUAA |
| | | U | | UAAUUAGGUGAGAAGGAUG |
| | | | | GUUGGGGGGGGUCGGCGUA |
| | | | | ACUCAGGGA |
| hsa-miR-8072 | 240 | GGCGGCGGGGA | 482 | GCGUCAAGAUGGCGGCGGG |
| | | GGUAGGCAG | | GAGGUAGGCAGAGCAGGAC |
| | | | | GCCGCUGCUGCCGCCGCCA |
| | | | | CCGCCGCCUCCGCUCCAGU |
| | | | | CGCC |
| hsa-miR-887-3p | 241 | GUGAACGGGCG | 483 | GUGCAGAUCCUUGGGAGCC |
| | | CCAUCCCGAGG | | CUGUUAGACUCUGGAUUUUA |
| | | | | CACUUGGAGUGAACGGGCG |
| | | | | CCAUCCCGAGGCUUUGCACA |
| | | | | G |
| hsa-miR-92b-5p | 242 | AGGGACGGGAC | 484 | CGGGCCCCGGGCGGGGGGG |
| | | GCGGUGCAGUG | | AGGGACGGGACGCGGUGCA |
| | | | | GUGUUGUUUUUUCCCCCGC |
| | | | | CAAUAUUGCACUCGUCCCGG |
| | | | | CCUCCGGCCCCCCCGGCCC |

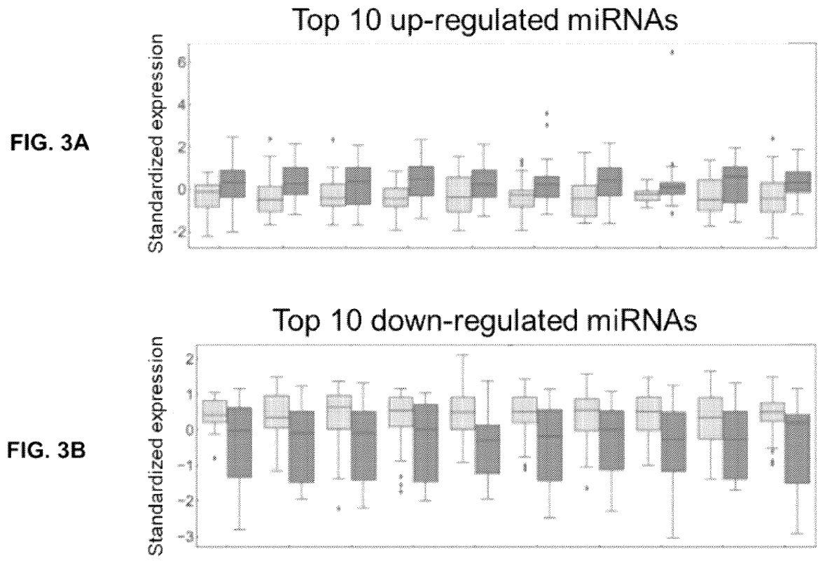

FIG. 3A shows the expression levels of top 10 up-regulated miRNAs (out of the 160 miRNAs shown in Table 2) in SLE patients (dark boxes) as compared with that in healthy donors (light boxes). These results suggest that up-regulation of these 10 miRNAs, may be correlated with SLE and, thus, may serve as biomarkers of SLE.

FIG. 3B shows the expression levels of top 10 down-regulated miRNAs (out of 82 miRNAs shown in Table 3) in SLE patients (dark boxes) as compared with that in healthy donors (light boxes). These results suggest that down-regulation of these 10 miRNAs, may be correlated with SLE and, thus, may also serve as biomarkers of SLE.

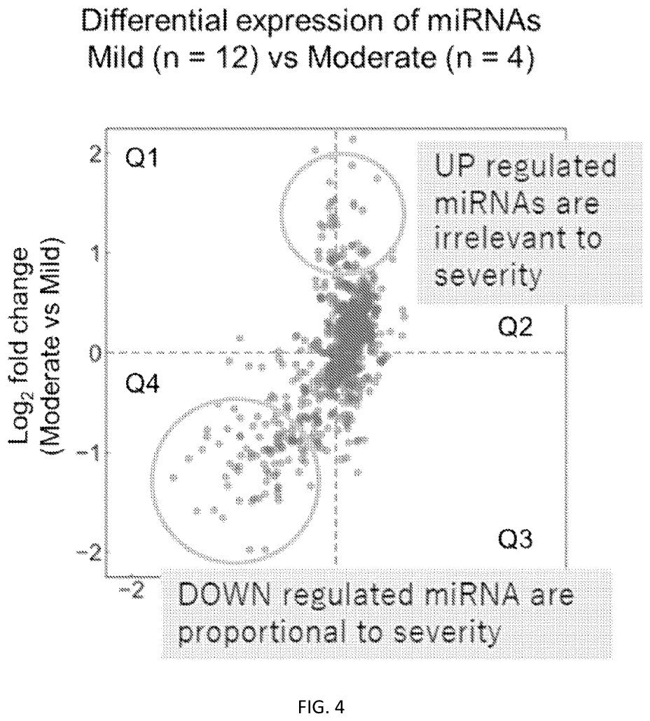

Identification of Urinary miRNAs as Biomarkers of SLE Severity FIG. 4 shows correlation of expression levels of each miRNA with SLE severity, e.g., moderate SLE versus mild SLE. The result shows that top down-regulated miRNAs (indicated by a circle) appear to be correlated with moderate SLE (Q4). In contrast, top up-regulated miRNAs (indicated by a circle) appear irrelevant to SLE severity because these up-regulated miRNAs appear to be associated with both moderate SLE (Q1) and mild SLE (Q2).

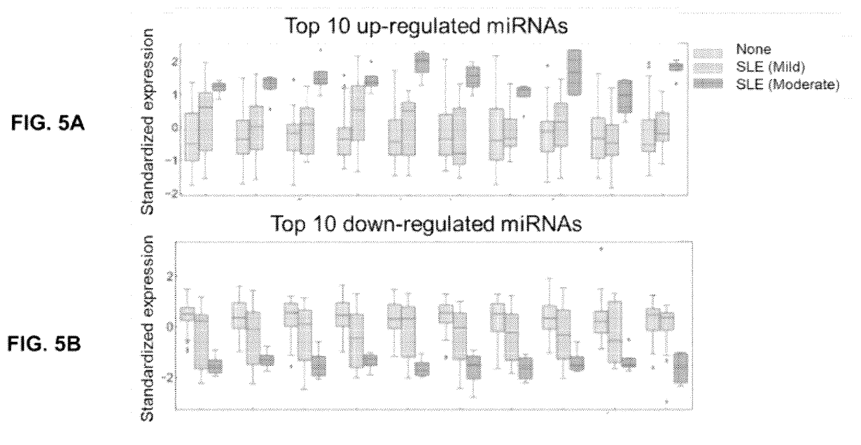

FIG. 5A shows the expression levels of top 10 up-regulated miRNAs associated with moderate SLE patients (dark boxes), mild SLE patients (light dark boxes), and healthy donors (light boxes). The expression levels of top 10 up-regulated miRNAs, appear significantly higher in moderate SLE patients than that in mild SLE patients. Thus, up-regulation of these miRNAs may serve as biomarkers of SLE severity, specifically for moderate SLE.

FIG. 5B shows the expression levels of top 10 down-regulated miRNAs in moderate SLE patients (dark boxes), mild SLE patients (light dark boxes), and healthy donors (light boxes). The expression levels of top 10 down-regulated miRNAs, appear significantly lower in moderate SLE patients than that in mild SLE patients. Thus, down-regulation of these miRNAs may serve as biomarkers of SLE severity, specifically for moderate SLE. Identification of Urinary miRNAs as Biomarkers of SLE Comorbidity

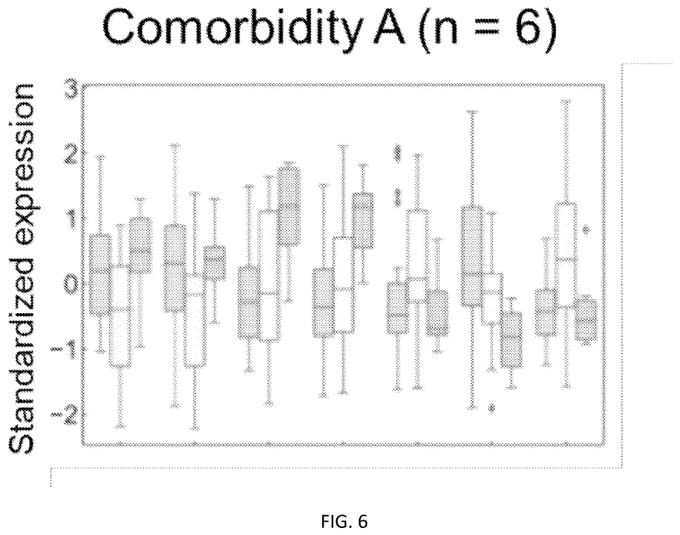

FIG. 6 shows up-regulation of 4 miRNAs and down-regulation of 3 miRNAs are correlated with SLE patients with comorbidity A (red boxes) as compared with SLE patients without comorbidity A (pink boxes). (n=6)

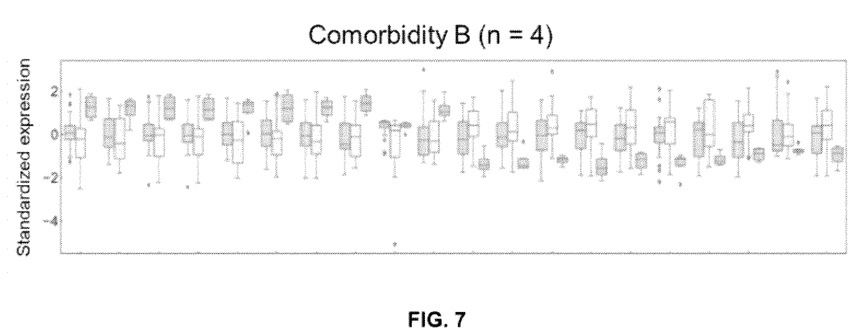

FIG. 7 shows up-regulation of 10 miRNAs and down-regulation of 10 miRNAs, are correlated with SLE patients with comorbidity B (red boxes) as compared with SLE patients without comorbidity B (pink boxes). (n=4)

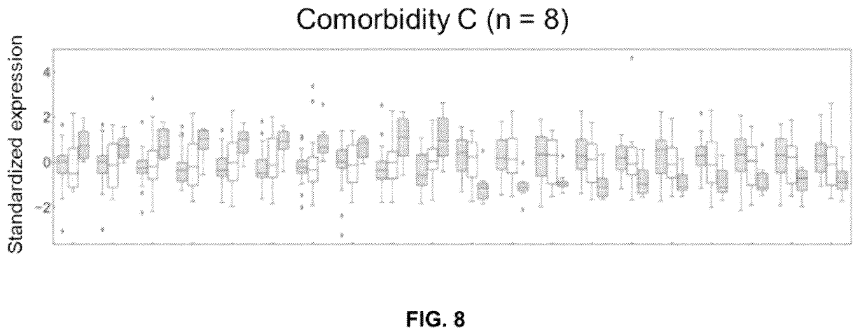

FIG. 8 shows up-regulation of 10 miRNAs, and down-regulation of 10 miRNAs, are correlated with SLE patients with comorbidity C (red boxes) as compared with SLE patients without comorbidity C (pink boxes). (n=8)

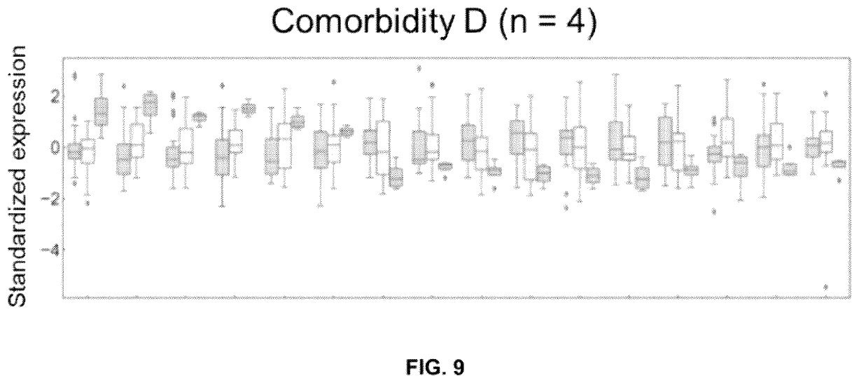

FIG. 9 shows up-regulation of 6 miRNAs, and down-regulation of 10 miRNAs, are correlated with SLE patients with comorbidity D (red boxes) as compared with SLE patients without comorbidity D (pink boxes). (n=4)

Advantages of the present disclosure may comprise collecting non-invasive samples, e.g., body fluids, from individuals for the isolation of miRNAs to be analyzed for the diagnosis of SLE, SLE severity, and SLE comorbidity. For individual suspected to have SLE, the presence or absence of SLE may be determined based on the individual's miRNA expression profiles. For SLE-positive individuals, the individuals' miRNA expression profiles may confirm SLE severity and SLE-associated comorbidities. The inventors surprising found that the use of body fluids and detection of miRNAs for the diagnosis of SLE was unconventional as compared to methods known in the art. Treatment plans may then be personalized based on these analyses.

### Example 2: Development of Classifier

The inventors developed a classifier to classify samples as indicative of SLE or free of SLE by comparing the values of individual miRNAs, e.g., expression levels. The inventors identified 484 miRNA sequences, SEQ ID Nos: 1-484. The inventors used the median of the miRNAs expression level of 60 samples as ‘cut off’, and if the value was higher/lower than the cutoff, the patient was classified as having SLE or not having SLE. Accuracy, sensitivity, specificity, AUC (area under the curve) are adopted from general metrics to evaluate the classifier based on simple cutoff.

242 miRNAs were significantly differentially expressed (160 up regulated, 82 down regulated) [p<0.05 T-test]. Down regulated miRNAs showed a trend to have larger fold change among cohorts. See FIG. 2. 160 miRNAs of the 242 showed significantly differentially expressed miRNAs. Differential expression analyses were conducted by comparing each miRNA signals from two groups. Fold change among cohorts plotted against p-value of t-test for each miRNA, and statistically significant miRNAs (p values<0.05) were selected as biomarker candidates.

The expression levels of each miRNA was compared to SLE disease severity. In FIG. 4, Expression levels of each miRNA were compared to SLE severity. The scatter plot of fold changes of each miRNAs (x-axis: SLE vs non-SLE, y-axis Moderate SLE vs Mild). FIG. 5A shows the top 10 up-regulated miRNAs and FIG. 5B shows the top 10 down-regulated miRNAs, as compared by no-disease, mild SLE, and moderate SLE.

Expression level were compared between SLE patients with and without the comorbidity. miRNAs with p<0.05 in t-test were selected as biomarker.

SLE Expression

Accuracy was calculated by the following equation based on classification the logistic regression model (True Positive+True Negative)/(True Positive+True Negative+False positive+False negative). Logistic regression model to estimate whether the sample if from SLE or not. The model was developed independently for each miRNA, and its expression level of each sample were used as features. The model was developed based on python sklearn (11 regularization, c=1, leave one out cross validation). To develop the classifiers, the inventors selected 5-20 random miRNAs from the set of 242 miRNAs and developed classifiers multiple times and evaluated the scores. miRNAs are randomly selected from the 242 miRNAs in the expression. The expression level of the selected miRNAs were used to develop the Logistic regression model. For each miRNA selection, classification was repeated 20 times. Accuracy, sensitivity and specificity, AUC are respective results achieved by developing a logistic regression model using the selected 5-20 miRNAs. Receiver Operating Characteristic (ROC) curve was plotted based on the raw values for the miRNA expression levels, and this represents the performance of the classifier.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A method for detecting miRNA, comprising
(a) obtaining a sample;
(b) capturing or isolating extracellular vesicles from the sample;
(c) disrupting the extracellular vesicles; and
(d) detecting the miRNA present in the sample.

2. The method of claim 1, wherein the miRNA is a ribonucleotide sequence selected from the group consisting of SEQ ID NO: 1-484 or a combination thereof.

3. The method of claim 1, wherein the isolation of the extracellular vesicles comprises capturing the extracellular vesicles on a nanowire.

4. A method for identifying a patient as having a marker correlated with systemic lupus erythematosus (SLE), comprising:
(a) obtaining a sample from a patient suspected of having SLE,
(b) analyzing miRNA expression in the obtained sample, and
(c) identifying the patient
(i) as having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample, or
(ii) as not having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

5. The method of claim 4, wherein a SLE severity is analyzed by:
(c) identifying the patient
(i) as having the marker correlated with moderate SLE if a decrease in expression of at least one miRNA selected from SEQ ID NOs: 161-242 and 403-484 compared to a body fluid sample obtained from a healthy individual is detected in the patient sample, or
(ii) as not having the marker correlated with moderate SLE if a decrease in expression of at least one miRNA selected from SEQ ID NOs: 161-242 and 403-484 compared to a body fluid sample obtained from a healthy individual fails to be detected.

6. The method of claim 4, wherein a comorbidity of SLE is analyzed by:
(c) identifying the patient
(i) as having the marker correlated with a comorbidity of SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual is detected in the patient sample, or
(ii) as not having the marker correlated with a comorbidity of SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

7. The method of claim 4, wherein the analyzing comprises generating an miRNA profile from the sample comprising:
(a) introducing the sample into a fluidic device comprising a nanowire,
(b) capturing extracellular vesicles in the sample on the nanowire,
(c) disrupting the captured extracellular vesicles,
(d) extracting at least one miRNA from the disrupted extracellular vesicles,
(e) detecting the extracted miRNA; and,
(f) analyzing the detected miRNA.

8. The method of claim 4, wherein the analyzing comprises:
(a) extracting extracellular vesicles from the obtained body fluid sample;
(b) analyzing oligonucleotide sequences of RNA included in the extracted extracellular vesicles; and
(c) generating an miRNA profile from the body fluid based on the analyzed sequences.

9. The method of claim 8, wherein the step (a) applies a fluidic device comprising a nanowire.

10. The method of claim 8, wherein the step (b) comprises:
purifying RNA from the extracted extracellular vesicles;
preparing a cDNA library of miRNA included in the purified RNA; and
analyzing oligonucleotide sequences of the cDNA library

11. The method of claim 4, wherein the sample is a body fluid.

12. The method of claim 11, wherein the body fluid is blood, urine, plasma, saliva, ascites, bronchoalveolar lavage fluid, cerebrospinal fluid, or a combination thereof.

13. The method of claim 4, wherein the method further comprises isolating the extracellular vesicle from the sample.

14. The method of claim 13, wherein the extracellular vesicle is isolated by differential ultracentrifugation, density gradient centrifugation, immunoaffinity, ultrafiltration, polymer-based precipitation, size-exclusion chromatography, or a combination thereof.

15. The method of claim 4, wherein an increase in expression of at least one miRNA selected from SEQ ID NOs: 1-160 and 243-402 and/or a decrease in expression of at least one miRNA selected from SEQ ID NOs: 161-242 and 403-484 compared to a sample obtained from a healthy individual is detected in the patient sample is indicative of the patient having systemic lupus erythematosus (SLE).

16. The method of claim 4, wherein as not having the marker correlated with SLE if an increase in expression of at least one miRNA selected from SEQ ID NOs: 1-160 and 243-402 and/or a decrease in expression of at least one miRNA selected from SEQ ID NOs: 161-242 and 403-484 compared to a body fluid sample obtained from a healthy individual fails to be detected.

17. The method of claim 4, wherein the nanowire comprises at least one positively charged surface selected from the group consisting of ZnO, SiO₂, Li₂O, MgO, Al₂O₃, CaO, TiO₂, Mn₂O₃, Fe₂O₃, CoO, NiO, CuO, Ga₂O₃, SrO, In₂O₃, SnO₂, Sm₂O₃, EuO, and combinations thereof.

18. The method of claim 4, wherein the nanowire is porous, magnetic, or both porous and magnetic.

19. The method of claim 4, wherein the length of the nanowire may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nanometers (nm).

20. The method of claim 4, wherein the length of the nanowire is between about 1 and 500 nm, 100 and 500 nm, 200 and 400 nm, 250 and 500 nm, 50 and 250 nm, 10 and 100 nm, 2 and 200 nm, 300 and 500 nm, 400 and 500 nm, 150 and 450 nm, 250 and 300 nm, 10 and 50 nm, 100 and 350 nm, 350 and 500 nm, or 200 and 300 nm.

21. The method of claim 4, wherein the cross-section of the nanowire is substantially circular, elliptical, regular polygonal, polygonal, hollow body.

22. The method of claim 4, wherein the outer shape of the nanowire may be substantially cylindrical, elliptical or polygonal.

23. The method of claim 4, wherein the nanowire is hollow or hollow bodies or may be substantially material-packed structures.

24. The method of claim 4, wherein the nanowire is formed of one material or a plurality of materials.

25. The method of claim 4, wherein the nanowire is coated on its surface with a coating material.

26. The method of claim 4, wherein the extracellular vesicles are disrupted by a cytolysis buffer.

27. The method of claim 26, wherein the extracellular vesicles are disrupted by alkali/detergent pre-treatment, storage at about −25° C., for about 1-10 days, optionally about 7 days, or a combination thereof.

28. The method of claim 4, wherein the extracting miRNAs is performed in situ.

29. The method of claim 4, wherein the extracellular vesicle is an exosome, microvesicle, apoptosis body, or a combination thereof.

30. The method of claim 4, wherein the sample is introduced into a device, optionally a microfluidic device, comprising:
(a) a sample input in fluid communication with
(b) a separation means, optionally a membrane, filter, at least one nanowire, or combination thereof, in fluid communication with
(c) a waste chamber or
(d) waste output.

31. The method of claim 4, wherein the sample is introduced into a device comprising a solid substrate comprising a plurality of wells, each well comprising at least one nanowire.

32. The method of claim 4, wherein the sample is introduced into a device comprising a solid substrate comprising a plurality of chambers, optionally in fluid communication with each other, each chamber comprising at least one nanowire.

33. The method of claim 4, wherein the device comprises a cover, optionally a removable cover.

34. The method of claim 6, wherein the SLE is associated with a comorbidity selected from the group consisting of cancer, a greater risk for cancer, cardiovascular, renal, liver, rheumatological disease, neurological diseases, hypothyroidism, psychosis, anaemia, and combinations thereof.

35. The method of claim 34, wherein the comorbidity is selected from the group consisting of cancer, a greater risk for cancer, cardiovascular, renal, liver, rheumatological disease, neurological diseases, hypothyroidism, psychosis, anaemia, and combinations thereof, if an increase in expression of at least one miRNA selected from SEQ ID NOs: 1-160 and 243-402 and/or a decrease in expression of at least one miRNA selected from SEQ ID NOs: 161-242 and 403-484.

36. A method of treating SLE comprising the identifying a patient as having a marker correlated with SLE of claim 4 and administering to the patient an effective amount of a compound selected from the group consisting of nonsteroidal anti-inflammatory drugs (NSAIDs), immunosuppressants, and anti-BLyS antibody.

37. The method of claim 7, wherein the detecting is performed by quantitative polymerase chain reaction (PCR), miRNA microarrays, next generation RNA sequencing (NGS), and/or multiplex miRNA profiling.
